(19) United States
(12) Patent Application Publication
(10) Pub. No.: US 2022/0412975 A1
(43) Pub. Date: Dec. 29, 2022

(54) CHARACTERIZING MODULATORS OF ADAMTS-7 AND ADAMTS-12

(71) Applicants: The Broad Institute, Inc., Cambridge, MA (US); Bayer Aktiengesellschaft, Leverkusen (DE)

(72) Inventors: Dmitry Zubov, Remscheid (DE); Bryan MacDonald, Cambridge, MA (US); Yi Xing, Cambridge, MA (US); Nadine Elowe, Cambridge, MA (US); Damian Brockschnieder, Haan (DE)

(21) Appl. No.: 17/776,424

(22) PCT Filed: Nov. 12, 2020
(86) PCT No.: PCT/US20/60159

§ 371 Date: May 12, 2022

Related U.S. Application Data
(60) Provisional application No. 63/068,570, filed on Aug. 21, 2020.
(60) Provisional application No. 63/062,964, filed on Aug. 7, 2020.
(60) Provisional application No. 62/935,832, filed on Nov. 15, 2019.

(51) Int. Cl. G01N 33/573; C12N 9/64; G01N 21/64

(52) U.S. Cl. CPC G01N 33/573; C12N 9/6489; G01N 21/6428; G01N 2333/8146; G01N 2021/6439

(57) ABSTRACT

Provided herein are assays for the detection and characterization of modulators of ADAMTS-7 and/or ADAMTS-12. Also provided herein are recombinant nucleic acids for the improved expression of functional metalloproteinases ADAMTS-7 and nucleic acids for the improved expression of functional metalloproteinases ADAMTS-12. In addition, provided herein are peptide substrates (e.g. FRET substrates) for use in assays for the detection and characterization of modulators of ADAMTS-7 and/or ADAMTS-12.

### RELATED APPLICATIONS

This application claims the benefit of priority to U.S. Provisional Patent Application No. 62/935,832 filed Nov. 15, 2019, U.S. Provisional Patent Application No. 63/062,964 filed Aug. 7, 2020 and U.S. Provisional Patent Application No. 63/068,570 filed Aug. 21, 2020.

### BACKGROUND

The process of vascular remodeling plays an important role in the pathogenesis of cardiovascular diseases and/or lung diseases as well as metabolic, inflammatory and/or vascular disease states including atherosclerosis and post-angioplasty restenosis (Wang et al., Sheng Li Xue Bao. 2010 Aug. 25; 62(4):285-94). Metalloproteinases such as the members of the ADAMTS family constitute one class of proteins which are involved in this process (Galis & Kathri, Circ Res., 2002 Feb. 22; 90(3):251-62). Accordingly, at least in the context of the aforementioned diseases or disease states, modulation of these metalloproteases may be beneficial for therapeutic intervention. However, various clinical trials using pan-metalloproteinase inhibitors have failed, e.g. due to severe side effects.

Thus, there is a need for selective modulators (e.g., agonists or inhibitors) of individual metalloproteases, or specific combinations of metalloproteases. However, development of (high throughput) assays to identify and characterize these modulators has been challenging so far. For example, while the reproducible and efficient expression and purification of recombinant functional protein has proven technically challenging for certain metalloproteinases, including ADAMTS-7 and ADAMTS-12. Moreover, the substrate specificities of ADAMTS-7 and ADAMTS-12 are poorly characterised. Only two proteins (cartilage oligomeric matrix protein (COMP) and thrombospondin 1 (TSP1)) have been reported as substrates for ADAMTS-7, without identification of the specific cleavage sites (Colige et al, Proteomic discovery of substrates of the cardiovascular protease ADAMTS-7, 2019, The Journal of Biological Chemistry 294, p. 8037-8045; Zhang et al. (2015) The Function and Roles of ADAMTS-7 in Inflammatory Diseases. Mediators Inflamm. 2015; 2015:801546. doi: 10.1155/2015/801546; Kessler et al. (2015) ADAMTS-7 inhibits re-endothelialization of injured arteries and promotes vascular remodeling through cleavage of thrombospondin-1. Circulation. 131:1191-201). Notably, even where a natural substrate has been identified, the specific cleavage site for the respective metalloproteinases is required to design assays and artificial substrates for the identification and characterization of metalloproteinase modulators.

Thus, there is a great need for compositions and methods that facilitate the characterization of modulators of metalloproteinases, including ADAMTS-7 and/or ADAMTS-12.

### SUMMARY

Provided herein are assays and reagents for the detection and characterization of ADAMTS-7 and/or ADAMTS-12 modulators, in particular, with regard to activity modulation and/or selectivity for specific metalloproteinase(s). Also provided are recombinant nucleic acids for expression of functional ADAMTS-7 and/or ADAMTS-12, as well as peptide substrates for ADAMTS-7 and/or ADAMTS-12, e.g. for use in such assays. Notably, embodiments of the provided assay were used to identify modulators of ADAMTS-7, such as potent and selective antagonists for ADAMTS-7, as well as multi-inhibitory molecules antagonizing two or more members of the ADAMTS family.

In some aspects, recombinant nucleic acids for expression of an ADAMTS-7 polypeptide that comprises a functional segment of a rodent prodomain of ADAMTS-7 as a first portion and a functional segment of human CD domain of ADAMTS-7 (e.g., a functional human ADAMTS-7 CD domain or catalytic domain) as a second portion are disclosed. The word “segment” as used in this specification includes less than full-length forms as well as the full-length forms of the indicated domains.

In some aspects, recombinant nucleic acids for expression of an ADAMTS-7 polypeptide are disclosed, in which the polypeptide includes a first portion having a sequence identity of >80% with the sequence of residues 1-217 of SEQ ID NO: 1 or with the sequence of residues 1-217 of SEQ ID NO: 2; and a second portion having a sequence identity of >80% with the sequence of residues 218-518 of SEQ ID NO: 1.

In some embodiments of these aspects, the first portion of the polypeptide includes residues 1-217 of SEQ ID NO: 1 or residues 1-217 of SEQ ID NO: 2, and/or the second portion amino acid sequence includes residues 218-518 of SEQ ID NO: 1.

In some aspects, recombinant nucleic acids for expression of an ADAMTS-7 polypeptide are disclosed, which encode for recombinant polypeptides that include a first portion having an amino acid sequence that aligns with a functional segment of an ADAMTS-7 prodomain amino acid sequence from a first species with a Needleman-Wunsch score greater than 700 when BLOSUM62 matrix, a gap opening penalty of 11, and a gap extension penalty of 1 are used; and a second portion having an amino acid sequence that aligns with a functional segment of an ADAMTS-7 CD domain amino acid sequence from a second species with a Needleman-Wunsch score greater than 1000 when BLOSUM62 matrix, a gap opening penalty of 11, and a gap extension penalty of 1 are used. In some embodiments, the first species is rat, and the second species is human. In some embodiments of these aspects, the second portion has an amino acid sequence that aligns with a functional segment of an ADAMTS-7 catalytic domain amino acid sequence from a second species with a Needleman-Wunsch score greater than 700 when BLOSUM62 matrix, a gap opening penalty of 11, and a gap extension penalty of 1 are used.

In certain embodiments of these aspects, the motif RQQR within the first portion of the polypeptide is altered, preferably into RQKR.

In some aspects, recombinant nucleic acids for expression of an ADAMTS-12 polypeptide that comprises a functional segment of a rodent prodomain of ADAMTS-12 as a first portion and a functional segment of human CD domain of ADAMTS-12 (e.g., a functional human ADAMTS-12 CD domain or catalytic domain) as a second portion are disclosed.

In certain aspects, recombinant nucleic acids for expression of an ADAMTS-12 polypeptide are disclosed, in which the polypeptide includes a first portion having a sequence identity of >80% with the sequence of residues 1-244 of SEQ ID NO: 15, and a second portion having a sequence identity of >80% with the sequence of residues 245-547 of SEQ ID NO: 15. In some embodiments, the first portion includes residues 1-244 of SEQ ID NO: 15, and/or the second portion amino acid sequence includes residues 245-547 of SEQ ID NO: 15.

In certain aspects, recombinant nucleic acids for expression of an ADAMTS-12 polypeptide are disclosed. The disclosed recombinant nucleic acids encode for a recombinant polypeptide that includes a first portion having an amino acid sequence that aligns with a functional segment of an ADAMTS-12 prodomain amino acid sequence from a first species with a Needleman-Wunsch score greater than 800 when BLOSUM62 matrix, a gap opening penalty of 11, and a gap extension penalty of 1 are used; and a second portion having an amino acid sequence that aligns with a functional segment of an ADAMTS12 CD domain amino acid sequence from a second species with a Needleman-Wunsch score greater than 1000 when BLOSUM62 matrix, a gap opening penalty of 11, and a gap extension penalty of 1 are used. In some embodiments, the first species is rat and the second species is human. In some embodiments of these aspects, the second portion has an amino acid sequence that aligns with a functional segment of an ADAMTS-12 catalytic domain amino acid sequence from a second species with a Needleman-Wunsch score greater than 700 when BLOSUM62 matrix, a gap opening penalty of 11, and a gap extension penalty of 1 are used.

In some embodiments of these aspects, the second portion includes an E397Q mutation with respect to the amino acid sequence of SEQ ID NO: 15 (e.g., the motif AHEL is mutated to AHQL). In certain embodiments, the encoded polypeptide or the second portion thereof is suited to cleave a peptide comprising standard residues 1-15 of the amino acid sequence of SEQ ID NO: 4, preferably with a k_{cat}/K_{M} of at least 20% of a corresponding k_{cat}/K_{M} of human ADAMTS-7 or human ADAMTS-12. In some embodiments, the recombinant polypeptide or fragment thereof is suited to cleave a peptide substrate comprising standard residues 1-15 of SEQ ID NO: 4.

In certain aspects, peptide substrates for ADAMTS-7 and/or ADAMTS-12 are disclosed. These peptide substrates include residues 1-15 of the amino acid sequence of SEQ ID NO: 4, or residues 1-15 of the amino acid sequence of SEQ ID NO: 5, or residues 1-13 of the amino acid sequence of SEQ ID NO: 8, or a fragment of any of the sequences according to a), b) or c), the fragment comprising the amino acid sequence EL.

In some embodiments, the peptide substrate includes a first moiety conjugated to a residue that is N-terminal to sequence fragment EL comprised within SEQ ID NO: 4,5, or 8 or the fragment thereof, and a second moiety conjugated to a residue that is C-terminal to said sequence fragment EL. In certain embodiments, the first moiety includes a fluorophore, and the second moiety includes a quencher, or the first moiety includes a quencher and the second moiety includes a fluorophore.

In some aspects, methods for the identification or characterization of an ADAMTS-7 and/or ADAMTS-12 modulator are disclosed. These methods include contacting an embodiment of the recombinant polypeptide or a fragment thereof with at least one test compound; contacting said recombinant polypeptide or fragment thereof with an embodiment of the peptide substrate that includes a fluorophore and a quencher; and detecting fluorescence as a measure for the activity of said recombinant polypeptide or a fragment thereof.

In some aspects, methods of using modulators of ADAMTS-7 and/or ADAMTS-12 identified by the disclosed methods for the identification or characterization of an ADAMTS-7 and/or ADAMTS-12 modulator are disclosed. These methods including using the modulators for treating coronary artery disease (CAD), peripheral vascular disease (PAD), or myocardial infarction (MI), for example by administering such a modulator to a subject that suffers from one or more of these conditions.

In some aspects, methods of producing embodiments of the disclosed recombinant polypeptides include cultivating a recombinant host cell including a disclosed embodiment of the recombinant nucleic acid; and recovering a disclosed embodiments of the recombinant polypeptide of a fragment thereof. Thus, in certain embodiments, provided herein are modulators of ADAMTS 7 and/or ADAMTS 12 identified according to a method provided herein, for use in the treatment of coronary artery disease (CAD), peripheral vascular disease (PAD), and/or myocardial infarction (ML). In some embodiments, provided herein is a method of treating CAD, PAD, and/or MI in a subject, the method comprising administering to the subject a modulator of ADAMTS-7 and/or ADAMTS-12 identified by a method provided herein.

In additional aspects, kits including a disclosed embodiment of the recombinant nucleic acid or a disclosed embodiment of the polypeptide and a disclosed embodiment of the peptide substrate are disclosed.

### BRIEF DESCRIPTION OF THE FIGURES

FIG. 1A— FIG. 1F show comparisons of recombinantly expressed wild type (WT) human ADAMTS-7, WT rat ADAMTS-7, and hybrid ADAMTS-7. Lane numbers of the SDS PAGE correspond with annotated SEC fractions. FIG. 1A and FIG. 1B show size exclusion (SEC) profile, SDS-page, and western blot (WB) analysis of human ADAMTS-7 (hADAMTS-7) (1-537 with respect to SEQ ID NO: 21) from mammalian cell culture. FIG. 1C and FIG. 1D show SEC profile, SDS-page, and WB analysis of rat ADAMTS-7 (rADAMTS-7) (1-575 with respect to SEQ ID NO: 22) from mammalian cell culture. FIG. 1E and FIG. 1F show the domain design of human and rat hybrid ADAMTS-7 (rPro-hCD; with respect to SEQ ID NOs: 22 and 21) with amino acid position indicated below, as well as SEC profile, SDS-page, and WB analysis of purified hybrid protein from mammalian cell culture. Abbreviations: rSP: rat signal peptide; rPro: rat propeptide (the rat prodomain without the signal peptide); hCatalytic: human catalytic domain; hDis: human disintegrin domain.

FIG. 2A-FIG. 2C show the primary structure and sequence comparison of ADAMTS-7. FIG. 2A shows the primary structure of hADAMTS-7 with residue numbers labeled at the domain boundaries (SP: signal peptide; Pro: propeptide (the Prodomain without the signal peptide); Dis: Disintegrin domain; TSR: thrombospondin repeat; PLAC: protease and lacunin domain). FIG. 2B and FIG. 2C show a sequence alignment of human and rat ADAMTS-7 in the Pro and CD domains (CD=catalytic+disintegrin). Different residues were colored in grey, and identical residues were black. Strongly similar residues were black marked with double dots and weakly similar residues were black marked with single dots. Sequence alignment was performed using CLUSTAWL.

FIG. 3A and FIG. 3B show two WT constructs for mammalian expression of ADAMTS-7 proteins. FIG. 3A shows that the SEC profile of hADAMTS-7 CD domain (residues 237-537 with respect to SEQ ID NO: 21) reveals production of soluble proteins (fractions B8-B12) are detectible only in the western blot. FIG. 3B shows that the SEC profile of hADAMTS-7 Pro-CD-TSR1 (residues 1-593 with respect to SEQ ID NO: 21, which includes the signal peptide as part of the prodomain) yielded little soluble proteins in the expected elution volumes underlined.

FIG. 4A and FIG. 4B shows that furin cleavage site (detailed in Example 3) mutants of ADAMTS-7 improved the yield of processed or unprocessed ADAMTS-7. FIG. 4A shows the mutations (grey) introduced into the sequence of the hybrid molecule rPro-hCD to change the furin cleavage efficiency during mammalian cell expression. Q216K mutation (with respect to SEQ ID NO: 1) in a furin cleavage site and a triple mutant R58A/R61A/R217A were named as rPro-hCD-FM2 (SEQ ID NO: 2) and rPro-hCD-3RA respectively. FIG. 4B shows that rPro-hCD-FM2 (SEQ ID NO: 2) improved the processing to generate more hCD domains during mammalian production and rPro-hCD-3RA abolished the processing to generate unprocessed protein only. Anti-His WB that targets the 6×His tag at the C-terminus of the protein was used to analyze the raw media of the mammalian cell culture two days post-transfection.

FIG. 5 shows an alignment between mouse ADAMTS-7 prodomain and rat ADAMTS-7 prodomain (with respect to SEQ ID NOs: 23 and 22, respectively, and excluding the signal peptide for both).

FIG. 6 shows that human ADAMTS-12 expression is improved when rat prodomain is used. The first gel was run under non-reducing conditions, whereas the second gel was run under reducing conditions. E393Q substitution (EQ) resulted in increased protein yield. The E393Q substitution is with respect to SEQ ID NO: 24 (with SEQ ID NO: 15 as a reference, the mutation corresponds to E397Q). The data was generated using Expi293 test expression, with 6 well-duplicate transfections, and with Western detection via Flag M2-HRP. As labeled in the figure, L237R is the ADAMTS12 optimized furin site.

FIG. 7A and FIG. 7B show FRET peptide substrate evaluation for TSP-1 and COMP candidate regions.

FIG. 8A-FIG. 8C: The top panels show curves following enzymatic activity over time with different substrates. The lower panels illustrate the rates of activity for rPro-hCD (SEQ ID NO: 1), rPro-hCD-FM2 (SEQ ID NO: 2) and rADAMTS-7(1-575) (SEQ ID NO: 3), respectively, using different substrates. TSP1-1 substrate (SEQ ID NO: 4) is most efficiently turned over by all ADAMTS-7 active constructs.

FIG. 9A-FIG. 9E: Generation and characterization of the mouse Adamts7 E373Q catalytic mutant allele. FIG. 9A, Structural context of the ADAMTS7 HExxH to HQxxH catalytic mutation. Crystal structures from ADAMTS4 WT (PDB:4WKI) (Durham et al. (2014) J Med Chem 57: 10476-10485) and ADAMTS4 EQ (PDB: 2RJP) (Mosyak et al. (2008) Protein Sci 17: 16-21) were aligned and annotated in PyMOL to highlight the catalytic residues and visualize the Zinc metal in the active site. The E to Q substitution preserves the tertiary structure of ADAMTS4 and the residues “VAHELGH” in the active site are conserved in ADAMTS7. FIG. 9B, Schematic of the Adamts7 E373Q catalytic mutant allele within exon 7. To generate the mutant allele, c. 1117G->C (p. E373Q) mutation at the Adamts7 catalytic domain and c. 1113C->T (p. A371A) to disrupt the PAM site were induced by CRISPR Homology-Directed Repair (HDR). FIG. 9C, Sanger sequencing of genomic tail DNA PCR and heart mRNA RT-PCR from WT and heterozygous+/E373Q mice. Representative forward reads show no evidence of allelic expression imbalance at the two nucleotide substitutions. FIG. 9D, Adamts7 mRNA expression level in the heart from WT, Adamts7−/− (KO) and homozygous E373Q/E373Q (EQ/EQ) mice were measured by real time quantitative polymerase chain reaction (qPCR) using 2 TaqMan probe sets (exon 4-5 boundary and exon 23-24 boundary). n=4 per group. FIG. 9E, Adamts12 mRNA expression levels were analyzed by real time qPCR in the heart and aorta harvested from WT, KO and EQ/EQ mice. n=4 per group.

FIG. 10A-FIG. 10C: Description of the mouse Adamts7 tm1b null allele and controls for X-gal staining during atherogenesis. FIG. 10A, Schematic of the Adamts7 tmal1 Knockout-first/conditional ready and Adamts7 tm1b Knockout with LacZ reporter alleles. Following Cre mediated recombination, the PGK-Neo cassette and exons 5 and 6 were deleted to generate the KO tm1b allele. FIG. 10B, Experimental design of the AAV-PCSK9 atherogenesis model. Adult 10-week-old mice were injected with rAAV8-D377Y-mPCSK9 and challenged a Western diet for 16 weeks. The mice were euthanized at 26 weeks of age for evaluation of atherosclerosis. FIG. 10C, X-gal staining in the aortic root plaque and heart from WT and +/tm1b mice to show negative and positive controls. WT and +/tm1b mice were injected with rAAV8-D377Y-mouse PCSK9 and fed western diet. Aortic roots were harvested at 8, 12 and 16 weeks after injection and were stained for β-galactosidase (β-gal) using X-gal staining.

FIG. 11A-FIG. 1111: Plasma PCSK9 levels in the AAV8-PCSK9 model resulting in decreased hepatic LDLR and increased plasma cholesterol. FIG. 11A-FIG. 11C, Measurements from littermate WT and KO mice and d-f, measurements from littermate WT and EQ/EQ mice. FIG. 11A, Plasma PCKS9 levels after injection (male, n=13 to 15 per group; female, n=17 to 21 per group). FIG. 11B, Liver lysates from indicated mice after 16 weeks of injection and non-injected mice were subjected to western blot analysis with anti-mouse LDLR and anti-β actin antibody. Error bars indicate s.e.m. FIG. 11C, Plasma total cholesterol levels after injection (male, n=13 to 15 per group; female, n=23 per group). FIG. 11D, Plasma PCKS9 levels after injection (male, n=13 to 14 per group; female, n=12 to 14 per group). FIG. 11E, Liver lysates from indicated mice after 16 weeks of injection and non-injected mice were subjected to western blot analysis with anti-mouse LDLR and anti-β actin antibody FIG. 11F, Plasma total cholesterol levels after injection (male, n=13 to 14 per group; female, n=12 to 14 per group). Error bars indicate s.e.m. FIG. 11G-FIG. 11H, Representative sections and quantitative analyses of CD68 positive macrophages (g, n=11 to 12 per group) and collagen (h, n=8 to 9 per group) in the aortic sinus.

FIG. 12A-FIG. 12F: Transient expression of ADAMTS7 during atherogenesis. ADAMTS7 expression reported by the LacZ gene trap tm1b allele in heterozygotes during atherogenesis at 6, 8, 12, and 16 weeks post infection with AAV8-PCSK9. FIG. 12A-FIG. 12D, Representative photomicrographs of X-gal staining in the aortic sinus at the indicated weeks after AAV8-PCSK9 injection. FIG. 12E, Stages examined in the atherogenic model post infection with AAV8-PCSK9 (data for 4, 10 and 14 weeks not shown). FIG. 12F, Quantitative analysis of X-gal positive cells in the plaque (n=3 to 5 per group, data points represent individual animals).

FIG. 13A-FIG. 13L: Decreased plaque formation in Adamts7 knockouts and in homozygous catalytic mutants in the AAV-PCSK9 atherogenic mouse model. At the age of 10 weeks, mice were injected with rAAV8-D377Y-mPCSK9 and challenged a western diet for an additional 16 weeks to stimulate atherosclerosis. The mice were euthanized at 26 weeks of age for evaluation of atherosclerosis in the aortic arch and aortic root. FIG. 13A-FIG. 13F, Representative photomicrographs of Oil-Red 0 staining and quantitative analysis of atherosclerotic lesion area in the aortas from littermate controls and Adamts7 knockouts (a-b, males, n=13 to 15 per group; females, n=17 to 23 per group). FIG. 13G-FIG. 13L, From a separate +/EQ×+/EQ cross, littermate controls and EQ/EQ homozygotes were evaluated for atherosclerosis (FIG. 13D and FIG. 13E, males, n=10 to 14 per group; females, n=11 to 14 per group). FIG. 13C, FIG. 13F, FIG. 13I, and FIG. 13L, Representative sections and quantitative analyses of a-SMA positive smooth muscle cells in the aortic sinus (n=10 to 12 per group). Data points represent individual animals, error bars indicate means±SEM, and a two-tailed Student's test was applied, *P<0.05, **P<0.01.

FIG. 14A-FIG. 14F: Adamts7 dosage and catalytic dependent effects on the ApoE KO atherogenic background. Mice from heterozygous intercrosses for the tm1b loss of function allele or the E373Q catalytic mutant allele, were challenged 10 weeks of high fat diet to stimulate atherosclerosis on the ApoE KO background. The mice were euthanized at 20 weeks of age for evaluation of atherosclerosis. FIG. 14A and FIG. 14D, Representative photomicrographs of Oil-Red 0 staining and quantitative analysis of atherosclerotic lesion area in the aortic arch. FIG. 14B, male WT vs Adamts7+/−(HET) vs Adamts7−/− (KO), n=7 to 18 per group; FIG. 14C, female WT vs HET vs KO, n=7 to 16 per group; FIG. 14E, male WT vs +/EQ vs EQ/EQ, n=9 to 11 per group; FIG. 14F, female WT vs +/EQ vs EQ/EQ, n=6 to 16 per each group. Data points represent individual animals, error bars indicate means±SEM, and one-way ANOVA with Tukey's test was applied, *P<0.05, **P<0.01.

FIG. 15A-FIG. 15F: No difference in aortic root lipid accumulation on the ApoE KO background. FIG. 15A, Representative photomicrographs of Oil-Red 0 staining from littermate WT, Adamts7+/−(HET) and Adamts7−/− (KO) on the ApoE KO atherogenic background. FIG. 15B and FIG. 15C, Quantitative analysis of atherosclerotic lesion area in the aortic sinus (FIG. 15B, males, WT n=11, HET n=15, KO n=7 per group;

FIG. 15C, females, WT n=9, HET n=10, KO n=7 per group). FIG. 15D, Representative photomicrographs of Oil-Red 0 staining from littermate WT, Adamts7+/EQ and Adamts7 EQ/EQ on the ApoE KO atherogenic background. FIG. 15E and FIG. 15F, Quantitative analysis of atherosclerotic lesion area in the aortic sinus (e, males, WT n=4, +/EQ n=9, EQ/EQ n=6 per group; f, females, WT n=2, +/EQ n=11, EQ/EQ n=5 per group).

FIG. 16A-FIG. 16C: Expression of ADAMTS7 and ADAMTS12 in primary mouse vascular smooth muscle cells. FIG. 16A, Adamts7 mRNA expression levels in primary VSMCs from WT, Adamts7−/− (KO) and Adamts7 E373Q/E373Q (EQ/EQ) mice were measured by real time qPCR using 2 TaqMan probe sets (exon 4-5 boundary and exon 23-24 boundary). n=4 per group. FIG. 16B, Adamts12 mRNA expression levels were analyzed by real time qPCR in primary VSMCs from WT, KO and EQ/EQ mice. n=4 per group. FIG. 16C, Immunofluorescent staining for a-SMA in primary mouse VSMCs. Primary mouse VSMCs harvested from Adamts7 KO mice were stained for a-SMA and Hoechst 33342.

FIG. 17A-FIG. 17F: ADAMTS7 catalytic function is responsible for ADAMTS7-mediated VSMC migration. FIG. 17A-FIG. 17D, Migration of primary VSMCs from Adamts7−/− (KO) and homozygous EQ/EQ catalytic mutant mice was assessed by wound healing assay. FIG. 17A and FIG. 17C, Representative photomicrographs of the wound at 0 and 12 hours in indicated genotype of VSMCs are shown. Solid line indicates wound edge. Dotted line indicates migration edge. FIG. 17B and FIG. 17D, The distance of migration is quantified at 4, 8 and 12 hours. n=8 to 9 per group. FIG. 17E, Representative images of Western blot in conditioned media and cell lysates from adenovirus-infected primary Adamts7 KO VSMCs. Cell lysates and conditioned media collected from Ad-Luciferase-, Ad-mAdamts7 (mAts7)-WT- and Ad-mAts7-373Q-infected primary Adamts7 KO VSMCs were subjected to western blot analysis with anti-FLAG antibody. FIG. 17F, Migration of primary Adamts7 KO VSMCs infected with Ad-Luciferase, Ad-mAdamts7 WT and Ad-E373Q was assessed by wound healing assay. The mean distance of migration is quantified at 4, 8 and 12 hours. n=8 to 9 per group. Error bars indicate means±SEM. Two-tailed Student's test was applied to FIG. 17D and FIG. 17E. *P<0.05, **P<0.01 ***P<0.001; one way ANOVA with Tukey's test was applied to FIG. 17A-FIG. 17C and FIG. 17F. FIG. 17A-FIG. 17C; **P<0.01, ***P<0.001, h; **P<0.01, ***P<0.001 (Ad-Luciferase vs Ad-mAts7 WT), ^{#}P<0.05 (Ad-mAts7-WT vs Ad-mAts7-373Q).

FIG. 18A-FIG. 18H: ADAMTS7 Ser214 CAD risk variant rs3825807 displays increased secretion and function compared to the Pro214 protective variant or catalytic mutant. FIG. 18A-FIG. 18C, Representative images and quantifications of western blots from HEK 293 cells transfected with empty vector control (VC), full-length ADAMTS7 risk variant Ser214 or Pro214. FIG. 18A, Full-length ADAMTS7 3×Flag protein was detected by anti-FLAG antibodies from conditioned media and cell lysates. FIG. 18B and FIG. 18C, Relative band intensities were quantified from n=4 per group. FIG. 18D-FIG. 18F, Representative images and quantifications of Western blot in conditioned media and cell lysates from adenovirus-infected HCA-SMCs infected with Ad-Luciferase (Luc), Ad-ADAMTS7-Ser214 or Ad-ADAMTS7-Pro214 were detected by Western blot analysis with anti-FLAG antibodies (n=5 per group). Error bars indicate means±SEM, two-tailed Student's test was applied to FIG. 18A-FIG. 18D, *P<0.05, ***P<0.001. g, Migration of primary Adamts7 KO VSMCs infected with Ad-Luciferase, Ad-ADAMTS7-Ser214 (WT), Ad-ADAMTS7-Pro214 or the Ad-ADAMTS7 catalytic mutant E389Q was assessed by wound healing assay. Representative photomicrographs of the wound at 0 and 12 hours in VSMCs infected with indicated adenovirus are shown. Solid line indicates wound edge. Dotted line indicates migration edge. FIG. 18H, Mean distance of migration is quantified at 4, 8 and 12 hours (n=8 to 9 per group, one-way ANOVA with Tukey's test, *P<0.05 for LUC vs Ser214).

### DETAILED DESCRIPTION

### General

Provided herein are methods and compositions for the detection and characterization of modulators of metalloproteases, including ADAMTS-7 and/or ADAMTS-12, in particular, with regard to activity modulation and/or selectivity for specific metalloproteinase(s). Also provided are recombinant nucleic acids for expression of functional ADAMTS-7 and/or ADAMTS-12, as well as peptide substrates for ADAMTS-7 and/or ADAMTS-12, e.g. for use in such assays.

ADAMTS (A Disintegrin And Metalloproteinase with ThromboSpondin motifs) constitute a family of metalloproteases. In humans, 19 secreted enzymes have been described. Different from other metalloproteinases, ADAMTS family members show a narrow substrate specificity (Wu et al., Eur J Med Res, 2015 Mar. 21; 20:27. doi: 10.1186/s40001-015-0118-4; Wang et al., Sheng Li Xue Bao. 2010 Aug. 25; 62(4):285-94).

ADAMTS play an important role in different processes such as assembly and degradation of extracellular matrix, angiogenesis, homeostasis, organogenesis, cancer, genetic disorders and arthritis (Zhang et al., Mediators Inflamm. 2015; 2015:801546. doi: 10.1155/2015/801546. Epub 2015 Nov. 30). The basic structure of ADAMTS comprises a metalloproteinase catalytic domain and a carboxy-terminal ancillary domain. The catalytic domain can be followed, for example at its carboxy-terminal side, by disintegrin-like domains. The catalytic domain and the disintegrin-like domains collectively are referred to as the “CD” domains in this disclosure. A prodomain can also be present, for example, at the N-terminal part of the encoded polypeptide. The prodomain can include a signal peptide, and the part of the prodomain without the signal peptide can be referred to as the propeptide. The prodomain can be cleaved off from the rest of ADAMTS, for example, to generate a functional ADAMTS. The consensus sequence HEXXHXBG(/N/S)BXHD (SEQ ID NO: 16) is shared as catalytic motif by ADAMTS with the three histidine residues coordinating a Zn²⁺-ion (Kelwick et al., Genome Biol., 2015 May 30; 16:113. doi: 10.1186/s13059-015-0676-3). The disintegrin-like domain may be involved in the regulation of ADAMTS activity by providing substrate-surfaces (Stanton et al., Biochim Biophys Acta., 2011 December; 1812(12):1616-29. doi: 10.1016/j.bbadis.2011.08.009. Epub 2011 Sep. 2). The ancillary domain is composed of one or more thrombospondin type 1 sequence repeats (TSRs), a cysteine-rich domain and a spacer domain. The ancillary domain furthermore determines substrate specificity as well as the localization of the protease and its interaction partners. Ancillary domains probably also have independent biological functions.

The 19 human ADAMTS proteases have been grouped into 8 clusters, largely on the basis of their known substrates. The first two clusters of the human ADAMTS proteases comprise the aggrecanases or proteoglycanases groups with ADAMTS-1, -4, -5, -8, -15, and ADAMTS-9 and -20, respectively. The third cluster, consisting of pro-collagen N-propeptidases, is constituted by ADAMTS-2, -3 and -14 which are crucially involved in the maturation of collagen-fibrils. The only member of the fourth cluster is ADAMTS-13, a protease that cleaves von-Willebrand factor. ADAMTS-7 and ADAMTS-12 are members of the fifth cluster. Members of this sub-group possess a mucin domain to which chondroitin sulfate chains are attached (Kelwick et al., Genome Biol., 2015 May 30; 16:113. doi: 10.1186/s13059-015-0676-3). For the three remaining clusters, the substrates are largely unknown. These clusters have been characterized by the organization of the domains of their respective ADAMTS-members. Each of these cluster contains two ADAMTS enzymes: ADAMTS-6 and -10, ADAMTS-16 and -18, and ADAMTS-17 and -19, respectively.

Natural substrates are presently known for only a handful of the ADAMs. They include: TNF for ADAM17/TACE, NOTCH for ADAM10/KUZ, procollagens I and II for ADAMTS2/procollagen N-proteinase (PNPI), aggrecan for ADAMTS4/aggrecanase-1 and ADAMTS5/aggrecanase-2.

The substrate specificity of ADAMTS-7 is poorly characterised. Only two proteins, cartilage oligomeric matrix protein (COMP) and thrombospondin 1 (TSP1) have previously been reported as substrates for ADAMTS-7, without identification of the specific cleavage sites (Colige et al, Proteomic discovery of substrates of the cardiovascular protease ADAMTS-7, 2019, The Journal of Biological Chemistry 294, p. 8037-8045; Zhang et al. (2015) The Function and Roles of ADAMTS-7 in Inflammatory Diseases. Mediators Inflamm. 2015; 2015:801546. doi: 10.1155/2015/801546; Kessler et al. (2015) ADAMTS-7 inhibits re-endothelialization of injured arteries and promotes vascular remodeling through cleavage of thrombospondin-1. Circulation. 131:1191-201).

ADAMTS-7 directly binds to and cleaves COMP (Liu et al., FASEB J. 2006 May; 20(7):988-90. Epub 2006 Apr. 3) with the catalytic domain of ADAMTS-7 being responsible for COMP-cleavage (Liu, Nat Clin Pract Rheumatol., 2009 January; 5(1):38-45. doi: 10.1038/ncprheum0961). Purified ADAMTS-7 protein was first reported to associate with and cleave COMP in a gel-based assay, producing a 100 kDa fragment from the pentameric 600 kDa COMP (Liu 2006 FASEB). Under reducing conditions, the monomeric COMP migrates at 120 kDa and was also shown to be converted into a 100 kDa fragment by ADAMTS-7, or by the paralog ADAMTS-12 (Luan 2008 Osteoarthritis Cartilage). Mapping of the ADAMTS-7 and COMP interaction regions defined the C-terminal TSRS-8 region of ADAMTS-7 and the four EGF repeat region of COMP (Liu 2006 FASEB).

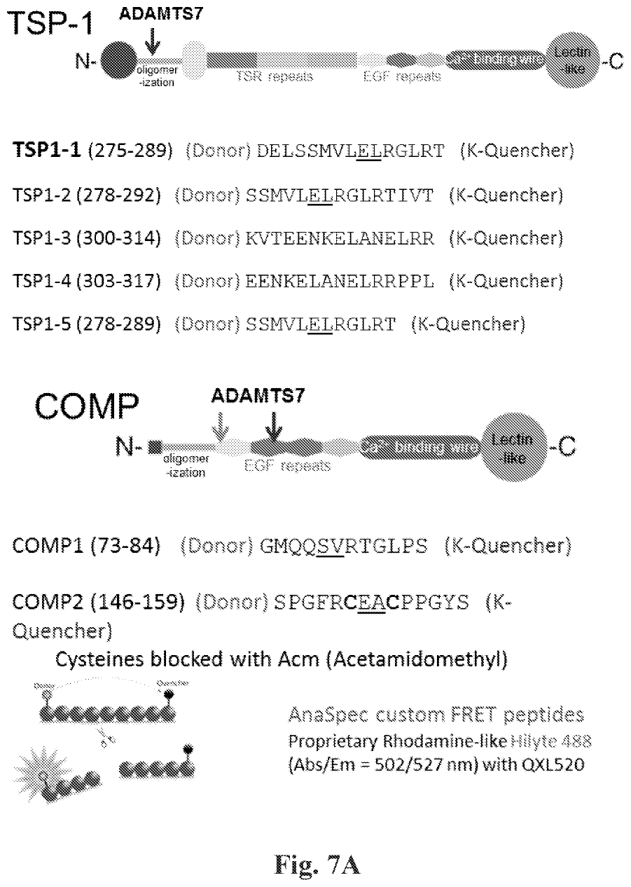

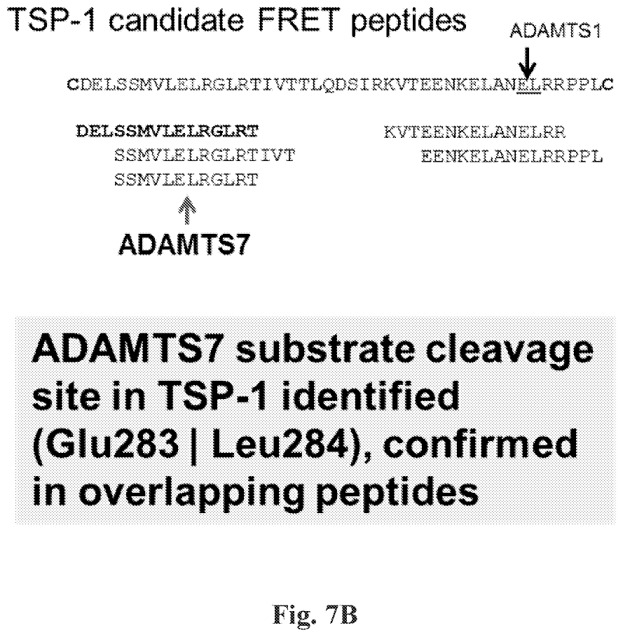

Another reported ADAMTS-7 substrate related to vascular disease is TSP1 (Kessler 2015 Circ Res). Interaction mapping again identified the ADAMTS-7 C-terminal TSRS-8 region as the substrate recognition site for TSP1. Monomeric TSP1 migrates at 170 kDa under reducing conditions and treatment with ADAMTS-7 resulted in a reported 140 kDa fragment (Kessler 2015 Circ Res). This placed the ADAMTS-7 cleavage site in TSP1 nearby what was previous described for ADAMTS1 at TSP1 E311⬇L312 in the context of E⬇LRR (Lee 2006 EMBO J) (See FIG. 7A and FIG. 7B). However, ADAMTS-7 was able to cleave TSP1 mutants E311D and L312I to produce the 140 kDa TSP1 fragment suggesting another substrate cleavage site (Kessler 2015 Circ Res). Many ADAMTS family members share a preference for glutamate at the P1 position, which is typically followed by an alanine or leucine in the P1′ (Kelwick 2015 Genome Bio). For instance, the well-studied aggrecanases ADAMTS4 and ADAMTS5 display a substrate cleavage site preference of E⬇LRG or E⬇ARG (Biniossek 2016 Mol Cell Proteomics). The latter is present in aggrecan at position E393⬇A394, although ADAMTS-7 was reported not to cleave this site (Sommerville 2004 JBC).

Even where a natural substrate has been identified, the specific cleavage site for the respective metalloproteinases is required to design assays and artificial substrates for the identification and characterization of metalloproteinase modulators. As described herein, these suitable cleavage sites have now been successfully identified for ADAMTS-7 and ADAMTS-12. Provided herein are short peptide substrates for ADAMTS-7 and ADAMTS-12, which can be used for the characterization of ADAMTS-7 or ADAMTS-12 activity. Furthermore, the provided peptide substrates can be used to identify and characterize modulators of ADAMTS-7 and/or ADAMTS-12, also in a high throughput assay.

In certain embodiments, selective modulators of ADAMTS-7 and/or its paralog ADAMTS-12 identified according to the methods provided herein may be used for the treatment of various diseases. ADAMTS-7 has been described as being involved in VSMC-calcification (Du et al., Arterioscler Thromb Vasc Biol., 2012 November; 32(11):2580-8. doi: 10.1161/ATVBAHA.112.300206. Epub 2012 Sep. 20). It contributes to oval cell activation and constitutes an important regulator in the context of biliary fibrosis. Furthermore, ADAMTS-7 has been reported to be involved in interactions of host and pathogen (Zhang et al., Mediators Inflamm. 2015; 2015:801546. doi: 10.1155/2015/801546. Epub 2015 Nov. 30. Both, ADAMTS-7 and ADAMTS-12 levels were significantly increased in cartilage and synovium of patients suffering from arthritis (Liu, Nat Clin Pract Rheumatol. 2009 January; 5(1):38-45. doi: 10.1038/ncprheum0961). ADAMTS-7 could also be detected in the urine of patients with different forms of cancer, such as prostate and bladder cancer (Roy et al., Clin Cancer Res. 2008 Oct. 15; 14(20):6610-7. doi: 10.1158/1078-0432.CCR-08-1136). Single nucleotide polymorphisms (SNPs) in the gene encoding for ADAMTS-7 have been linked to keratoconus with cataract, an ophthalmological disease (Dash et al., Mol. Vis. 2006 May 12; 12:499-505). Additionally, ADAMTS-7 expression was increased in patients with aortic aneurysms while COMP levels were markedly reduced in this patient group (Qin et al., Mol Med Rep., 2017 October; 16(4):5459-5463. doi: 10.3892/mmr.2017.7293. Epub 2017 Aug. 21).

Genome-wide association studies and meta-analyses thereof have suggested a correlation between ADAMTS-7 genetic variants and coronary artery disease (CAD) (Reilly et al., Lancet. 2011 Jan. 29; 377(9763):383-92. doi: 10.1016/S0140-6736(10)61996-4. Epub 2011 Jan. 14). An association between ADAMTS-7 and CAD was further supported by a meta-analysis of 14 genome-wide association studies (Schunkert et al., Nat. Genet. 2011 Mar. 6; 43(4):333-8. doi: 10.1038/ng.784). Genome-wide association studies have also proposed an association between ADAMTS-7 and myocardial infarction (MI) (Chan et al., J Am Heart Assoc., 2017 Oct. 31; 6(11). pii: e006928. doi: 10.1161/JAHA.117.006928).

In 2010 Wang et al. showed an increased expression of ADAMTS-7 in early- and late-stage human atherosclerotic plaques (Wang et al., Sheng Li Xue Bao. 2010 Aug. 25; 62(4):285-94), while Bauer et al. found in 2015 that knockout of Adamts-7 resulted in reduced atherosclerosis in mice (Bauer et al. (2015) Knockout of ADAMTS-7, a novel coronary artery disease locus in humans, reduces atherosclerosis in mice. Circulation 131:1202-1213).

ADAMTS-7 has been described to be involved in the migration of smooth muscle cells (SMC) and the development of neointimal hyperplasia. Immunohistochemistry showed co-localisation of ADAMTS-7 with vascular smooth muscle cells (VSMC) within the neointima (Wang et al., Circ Res., 2009 Mar. 13; 104(5):688-98. doi: 10.1161/CIRCRESAHA.108.188425. Epub 2009 Jan. 22). Formation of a neointima is characterized by a media-to-intima migration of VSMC and their subsequent proliferation. Interestingly, both migration as well as proliferation of VSMC were accelerated by ADAMTS-7 in vitro (Wang et al., Sheng Li Xue Bao. 2010 Aug. 25; 62(4):285-94). In line with these observations, overexpression of ADAMTS-7 led to an increased neointima formation in vivo, whereas knockdown reduced/delayed injury-induced neointimal hyperplasia (Wang et al, Circ Res., 2009 Mar. 13; 104(5):688-98. doi: 10.1161/CIRCRESAHA.108.188425. Epub 2009 Jan. 22). Kessler et al. could finally show that ADAMTS-7 knockouts exhibited increased re-endothelization and reduced neointima formation after balloon-wire injury (Kessler et al. (2015) ADAMTS-7 inhibits re-endothelialization of injured arteries and promotes vascular remodeling through cleavage of thrombospondin-1. Circulation. 131:1191-201).

One of the leading SNPs in the ADAMTS-7 locus, rs3825807, shows an adenine (A)-to-guanine (G) exchange in the ADAMTS-7 gene with the G-allele being associated with a reduced risk for CAD. The above-mentioned SNP (Single Nucleotide Polymorphism) leads to an amino acid exchange in the pro-domain of ADAMTS-7 and thereby has an impact on the maturation of ADAMTS-7. Furthermore, it has been described that the above mentioned SNP finds itself in a linkage disequilibrium with further SNPs within the ADAMTS-7 locus, such as rs1994016 and rs7178051 (Chan et al., J Am Heart Assoc., 2017 Oct. 31; 6(11). pii: e006928. doi: 10.1161/JAHA.117.006928). Supporting the aforementioned notions, rs3825807 has also been described to be associated with the susceptibility towards CAD in a chinese population and this association persisted even after correction for clinical co-variates (You et al., Mol Genet. Genomics. 2016 February; 291(1):121-8. doi: 10.1007/s00438-015-1092-9. Epub 2015 Jul. 19).

These observations are consistent with the findings of genome wide association studies, wherein carriers of SNP rs3825807 displayed a decreased protease activity of ADAMTS-7 and an improved survival for CAD (Bayoglu et al. (2018) Genetic variants rs1994016 and rs3825807 in ADAMTS-7 affect its mRNA expression in atherosclerotic occlusive peripheral arterial disease. J Clin Lab Anal. 32(1)).

### Sequences

Where the sequence information or listing apart from the amino acid sequence comprises modifications, fluorophores and/or quencher (e.g., within the feature data), these shall not be read restrictively but only in an exemplary way.

TABLE 1

| SEQ ID | | |
|---|---|---|
| NO: | VARIANT | TYPE |
| 1 | rPro-hCD (Rat 1-217/Human 237-537)-TEV-2Strep-6His | PRT |
| | MHRGLNLLLILCALAPHVLGPASGLPTEGRAGLDIVHPVRVDAGGSFLSYELWPRVLRKRDV | |
| | SAAQASSAFYQLQYQGRELLFNLTTNPYLLAPGFVSEIRRRSNLSNVHIQTSVPTCHLLGDV | |
| | QDPELEGGFAAISACDGLRGVFQLSNEDYFIEPLDEVPAQPGHAQPHMVYKHKRSGQQDDSR | |
| | TSGTCGVQGSPELKHQREHWEQRQQKRRQQRSVSKEKWVETLVVADAKMVEYHGQPQVESYV | |
| | LTIMNMVAGLFHDPSIGNPIHITIVRLVLLEDEEEDLKITHHADNTLKSFCKWQKSINMKGD | |
| | AHPLHHDTAILLTRKDLCAAMNRPCETLGLSHVAGMCQPHRSCSINEDTGLPLAFTVAHELG | |
| | HSFGIQHDGSGNDCEPVGKRPFIMSPQLLYDAAPLTWSRCSRQYITRFLDRGWGLCLDDPPA | |
| | KDIIDFPSVPPGVLYDVSHQCRLQYGAYSAFCEDMDNVCHTLWCSVGTTCHSKLDAAVDGTR | |
| | CGENKWCLSGECVPVGFRPEAVGSENLYFQSGWSHPQFEKGGGSGGGSGGGSWSHPQFEKHH | |
| | HHHH | |
| 2 | rPro-hCD-FM2 (Rat 1-217/Human 237-537 FM2 (Q216K))-TEV-2Strep- | PRT |
| | 6His | |
| | MHRGLNLLLILCALAPHVLGPASGLPTEGRAGLDIVHPVRVDAGGSFLSYELWPRVLRKRDV | |
| | SAAQASSAFYQLQYQGRELLFNLTTNPYLLAPGFVSEIRRRSNLSNVHIQTSVPTCHLLGDV | |
| | QDPELEGGFAAISACDGLRGVFQLSNEDYFIEPLDEVPAQPGHAQPHMVYKHKRSGQQDDSR | |
| | TSGTCGVQGSPELKHQREHWEQRQQKRRQKRSVSKEKWVETLVVADAKMVEYHGQPQVESYV | |
| | LTIMNMVAGLFHDPSIGNPIHITIVRLVLLEDEEEDLKITHHADNTLKSFCKWQKSINMKGD | |
| | AHPLHHDTAILLTRKDLCAAMNRPCETLGLSHVAGMCQPHRSCSINEDTGLPLAFTVAHELG | |
| | HSFGIQHDGSGNDCEPVGKRPFIMSPQLLYDAAPLTWSRCSRQYITRFLDRGWGLCLDDPPA | |
| | KDIIDFPSVPPGVLYDVSHQCRLQYGAYSAFCEDMDNVCHTLWCSVGTTCHSKLDAAVDGTR | |
| | CGENKWCLSGECVPVGFRPEAVGSENLYFQSGWSHPQFEKGGGSGGGSGGGSWSHPQFEKHH | |
| | HHHH | |
| 3 | rADAMTS-7 (Rat 1-575)-Flag | PRT |
| | MHRGLNLLLILCALAPHVLGPASGLPTEGRAGLDIVHPVRVDAGGSFLSYELWPRVLRKRDV | |
| | SAAQASSAFYQLQYQGRELLFNLTTNPYLLAPGFVSEIRRRSNLSNVHIQTSVPTCHLLGDV | |
| | QDPELEGGFAAISACDGLRGVFQLSNEDYFIEPLDEVPAQPGHAQPHMVYKHKRSGQQDDSR | |
| | TSGTCGVQGSPELKHQREHWEQRQQKRRQQRSISKEKWVETLVVADSKMVEYHGQPQVESYV | |
| | LTIMNMVAGLYHDPSIGNPIHITVVRLIILEDEEKDLKITHHADDTLKNFCRWQKNVNMKGD | |
| | DHPQHHDTAILLTRKDLCATMNHPCETLGLSHVAGLCHPQLSCSVSEDTGLPLAFTVAHELG | |
| | HSFGIQHDGTGNDCESIGKRPFIMSPQLLYDRGIPLTWSRCSREYITRFLDRGWGLCLDDRP | |
| | SKGVINFPSVLPGVLYDVNHQCRLQYGPSSAYCEDVDNVCYTLWCSVGTTCHSKMDAAVDGT | |
| | SCGKNKWCLNGECVPEGFQPETVDGGWSGWSAWSVCSRSCGVGVRSSERQCTQPVPKNKGKY | |
| | CVGERKRYRLCNLQACPENLYFQGDYKDDDDK | |
| 4 | Peptide Substrate for ADAMTS-7/12 | PRT |
| | (HiLyteFluor-488)-DELSSMVLELRGLRT-K(QXL520)-NH2 | |
| 5 | Peptide Substrate for ADAMTS-7/12 | PRT |
| | (HiLyteFluor-488)-SSMVLELRGLRTIVT-K(QXL520)-NH2 | |
| 6 | Peptide Substrate for ADAMTS-7/12 | PRT |
| | (HiLyteFluor-488)-KVTEENKELANELRR-K (QXL520)-NH2 | |
| 7 | Peptide Substrate for ADAMTS-7/12 | PRT |
| | (HiLyteFluor-488)-EENKELANELRRPPL-K (QXL520)-NH2 | |
| 8 | Peptide Substrate for ADAMTS-7/12 | PRT |
| | (HiLyteFluor-488)-SSMVLELRGLRT-K(QXL520)-NH2 | |
| 9 | Peptide Substrate for ADAMTS-7/12 | PRT |
| | (Donor) GMQQSVRTGLPS (K-Quencher) | |
| 10 | Peptide Substrate for ADAMTS-7/12 | PRT |
| | (Donor) SPGFRCEACPPGYS (K-Quencher) | |
| 11 | Peptide Substrate for ADAMTS-7 and ADAMTS-12 | PRT |
| | (HiLyteFluor-488)-DELSSMVLELRGLRT-K(QXL520)-E-NH2 | |
| 12 | Peptide Substrate for ADAMTS-7/12 | PRT |
| | (HiLyteFluor-488)-DELSSMVLELRGLRT-K(QXL520)-K-NH2 | |
| 13 | Peptide Substrate for ADAMTS-7/12 | PRT |
| | (HiLyteFluor-488)-DELSSMVLELRGLRT-K(QXL520)-OH | |
| 14 | Peptide Substrate for ADAMTS4 and for ADAMTS5 | PRT |
| | Dabcyl-EEVKAKVQPY-Glu(Edans)-NH2 | |
| 15 | rPro-hCD for ADAMTS-12 | PRT |
| | MPCAQGNWMAKLSMVAQLLNFGAFCHGRQAQPWPVRFPDPKQEHFIKSLPEYHIVSPVQVDA | |
| | SGHFLSYGLHHPVTGSRKKRAAGGSGDQVYYRISHEEKNLFFNLTVNWEFLSNGYVVERRYG | |
| | NLSHVKMAASSGQPCHLRGTVLQQGPTIRMGTAALSACQGLTGFFHLPHGDFFIEPVKKHPL | |
| | TEEGYQPHVIYRRQSYRVPETKEPTCGLKDSLDNSVKQELQREKWERKNWPSRSLSRRSISK | |
| | ERWVETLVVADTKMIEYHGSENVESYILTIMNMVTGLFHNPSIGNAIHIVVVRLILLEEEEQ | |
| | GLKIVHHAEKTLSSFCKWQKSINPKSDLNPVHHDVAVLLTRKDICAGFNRPCETLGLSHLSG | |
| | MCQPHRSCNINEDSGLPLAFTIAHELGHSFGIQHDGKENDCEPVGRHPYIMSRQLQYDPTPL | |
| | TWSKCSEEYITRFLDRGWGFCLDDIPKKKGLKSKVIAPGVIYDVHHQCQLQYGPNATFCQEV | |
| | ENVCQTLWCSVKGFCRSKLDAAADGTQCGEKKWCMAGKCITVGKKPESIPGGGGSDYKDHDG | |
| | DYKDHDIDYKDDDDK | |
| 16 | ADAMTS Catalytic Motif Consensus Sequence | PRT |
| | HEXXHXBG (/N/S) BXHD | |
| 17 | Peptide Substrate for MMP12 | PRT |
| | Mca-Pro-Leu-Gly-Leu-Glu-Glu-Ala-Dap(Dnp)-NH2 | |
| 18 | Peptide Substrate for MMP15 | PRT |
| | MCA-Lys-Pro-Leu-Gly-Leu-DPA-Ala-Arg-NH2 | |
| 19 | Peptide Substrate for MMP2 | PRT |
| | MCA-Pro-Leu-Gly-Leu-DPA-Ala-Arg-NH2 | |
| 20 | Peptide Substrate for ADAM17 | PRT |
| | Mca-Pro-Leu-Ala-Gln-Ala-Val-Dap(Dnp)-Arg-Ser-Ser-Ser-Arg-NH2 | |
| 21 | Human ADAMTS-7 from Q9UKP4 | PRT |
| | MPGGPSPRSPAPLLRPLLLLLCALAPGAPGPAPGRATEGRAALDIVHPVRVDAGGSFLSY | |
| | ELWPRALRKRDVSVRRDAPAFYELQYRGRELRFNLTANQHLLAPGFVSETRRRGGLGRAH | |
| | IRAHTPACHLLGEVQDPELEGGLAAISACDGLKGVFQLSNEDYFIEPLDSAPARPGHAQP | |
| | HVVYKRQAPERLAQRGDSSAPSTCGVQVYPELESRRERWEQRQQWRRPRLRRLHQRSVSK | |
| | EKWVETLVVADAKMVEYHGQPQVESYVLTIMNMVAGLFHDPSIGNPIHITIVRLVLLEDE | |
| | EEDLKITHHADNTLKSFCKWQKSINMKGDAHPLHHDTAILLTRKDLCAAMNRPCETLGLS | |
| | HVAGMCQPHRSCSINEDTGLPLAFTVAHELGHSFGIQHDGSGNDCEPVGKRPFIMSPQLL | |
| | YDAAPLTWSRCSRQYITRFLDRGWGLCLDDPPAKDIIDFPSVPPGVLYDVSHQCRLQYGA | |
| | YSAFCEDMDNVCHTLWCSVGTTCHSKLDAAVDGTRCGENKWCLSGECVPVGFRPEAVDGG | |
| | WSGWSAWSICSRSCGMGVQSAERQCTQPTPKYKGRYCVGERKRFRLCNLQACPAGRPSFR | |
| | HVQCSHFDAMLYKGQLHTWVPVVNDVNPCELHCRPANEYFAEKLRDAVVDGTPCYQVRAS | |
| | RDLCINGICKNVGCDFEIDSGAMEDRCGVCHGNGSTCHTVSGTFEEAEGLGYVDVGLIPA | |
| | GAREIRIQEVAEAANFLALRSEDPEKYFLNGGWTIQWNGDYQVAGTTFTYARRGNWENLT | |
| | SPGPTKEPVWIQLLFQESNPGVHYEYTIHREAGGHDEVPPPVFSWHYGPWTKCTVTCGRG | |
| | VQRQNVYCLERQAGPVDEEHCDPLGRPDDQQRKCSEQPCPARWWAGEWQLCSSSCGPGGL | |
| | SRRAVLCIRSVGLDEQSALEPPACEHLPRPPTETPCNRHVPCPATWAVGNWSQCSVTCGE | |
| | GTQRRNVLCTNDTGVPCDEAQQPASEVTCSLPLCRWPLGTLGPEGSGSGSSSHELFNEAD | |
| | FIPHHLAPRPSPASSPKPGTMGNAIEEEAPELDLPGPVFVDDFYYDYNFINFHEDLSYGP | |
| | SEEPDLDLAGTGDRTPPPHSHPAAPSTGSPVPATEPPAAKEEGVLGPWSPSPWPSQAGRS | |
| | PPPPSEQTPGNPLINFLPEEDTPIGAPDLGLPSLSWPRVSTDGLQTPATPESQNDFPVGK | |
| | DSQSQLPPPWRDRTNEVFKDDEEPKGRGAPHLPPRPSSTLPPLSPVGSTHSSPSPDVAEL | |
| | WTGGTVAWEPALEGGLGPVDSELWPTVGVASLLPPPIAPLPEMKVRDSSLEPGTPSFPTP | |
| | GPGSWDLQTVAVWGTFLPTTLTGLGHMPEPALNPGPKGQPESLSPEVPLSSRLLSTPAWD | |
| | SPANSHRVPETQPLAPSLAEAGPPADPLVVRNAGWQAGNWSECSTTCGLGAVWRPVRCSS | |
| | GRDEDCAPAGRPQPARRCHLRPCATWHSGNWSKCSRSCGGGSSVRDVQCVDTRDLRPLRP | |
| | FHCQPGPAKPPAHRPCGAQPCLSWYTSSWRECSEACGGGEQQRLVTCPEPGLCEEALRPN | |
| | TTRPCNTHPCTQWVVGPWGQCSGPCGGGVQRRLVKCVNTQTGLPEEDSDQCGHEAWPESS | |
| | RPCGTEDCEPVEPPRCERDRLSFGFCETLRLLGRCQLPTIRTQCCRSCSPPSHGAPSRGH | |
| | QRVARR | |
| 22 | Rat ADAMTS-7 from Q1EHB3 | PRT |
| | MHRGLNLLLILCALAPHVLGPASGLPTEGRAGLDIVHPVRVDAGGSFLSYELWPRVLRKR | |
| | DVSAAQASSAFYQLQYQGRELLFNLTTNPYLLAPGFVSEIRRRSNLSNVHIQTSVPTCHL | |
| | LGDVQDPELEGGFAAISACDGLRGVFQLSNEDYFIEPLDEVPAQPGHAQPHMVYKHKRSG | |
| | QQDDSRTSGTCGVQGSPELKHQREHWEQRQQKRRQQRSISKEKWVETLVVADSKMVEYHG | |
| | QPQVESYVLTIMNMVAGLYHDPSIGNPIHITVVRLIILEDEEKDLKITHHADDTLKNFCR | |
| | WQKNVNMKGDDHPQHHDTAILLTRKDLCATMNHPCETLGLSHVAGLCHPQLSCSVSEDTG | |
| | LPLAFTVAHELGHSFGIQHDGTGNDCESIGKRPFIMSPQLLYDRGIPLTWSRCSREYITR | |
| | FLDRGWGLCLDDRPSKGVINFPSVLPGVLYDVNHQCRLQYGPSSAYCEDVDNVCYTLWCS | |
| | VGTTCHSKMDAAVDGTSCGKNKWCLNGECVPEGFQPETVDGGWSGWSAWSVCSRSCGVGV | |
| | RSSERQCTQPVPKNKGKYCVGERKRYRLCNLQACPPDRPSFRHTQCSQFDSMLYKGKLHK | |
| | WVPVLNDENPCELHCRPFNYSNREKLRDAVMDGTPCYQGRISRDICIDGICKKVGCDFEL | |
| | DSGAEEDRCGVCRGDGSTCHTVSRTFKEAEGMGYVDVGLIPAGAREILIEEVAEAANFLA | |
| | LRSEDPDKYFLNGGWTIQWNGDYQVAGTTFTYTRKGNWETLTSPGPTTEPVWIQLLFQER | |
| | NPGVHYKYTIQRASHSEAQPPEFSWHYGPWSKCPVTCGTGVQRQSLYCMEKQAGIVDEGH | |
| | CDHLSRPRDRKRKCNEEPCPARWWVGDWQPCSRSCGPGGFFRRAVFCTRSVGLDEQRALE | |
| | PSACGHLPRPLAEIPCYHYVACPSSWGVGNWSQCSVTCGAGIRQRSVLCINNTGVPCDGA | |
| | ERPITETFCFLQPCQYSTYIVDTGASGSGSSSPELFNEVDFDPHQPVPRPSPASSPKPVS | |
| | ISNAIDEEDPELDPPGPVFVDDFYYDYNFINFHEDLSYGSFEESHSDLVDIGGQTVPPHI | |
| | RPTEPPSDSPVPTAGAPGAEEEGIQGSWSPSPLLSEASHSPPVLLENTPVNPLANFLTEE | |
| | ESPIGAPELGLPSVSWPPASVDGMVTSVAPGNPDELLVREDTQSQPSTPWSDRNKLSKDG | |
| | NPLGPTSPALPKSPFPTQPSSPSNSTTQASLSPDAVEVSTGWNVALDPVLEADLKPVHAP | |
| | TDPGLLDQIQTPHTEGTQSPGLLPRPAQETQTNSSKDPAVQPLQPSLVEDGAPTDLLPAK | |
| | NASWQVGNWSQCSTTCGLGAIWRLVRCSSGNDEDCTLSSRPQPARHCHLRPCAAWRAGNW | |
| | SKCSRNCGGGSATRDVQCVDTRDLRPLRPFHCQPGPTKPPTRQLCGTQPCLPWYTSSWRE | |
| | CSEACGGGEQQRLVTCPEPGLCEESLRPNNTRPCNTHPCTQWVVGPWGQCSAPCGGGVQR | |
| | RLVKCVNTQTGLAEEDSDLCSHEAWPESSRPCATEDCELVEPSRCERDRLPFNFCETLRL | |
| | LGRCQLPTIRAQCCRSCPPLSRGVPSRGHQRVARR | |
| 23 | Mouse ADAMTS-7 from Q68SA9 | PRT |
| | MHRGPSLLLILCALASRVLGPASGLVTEGRAGLDIVHPVRVDAGGSFLSYELWPRVLRKR | |
| | DVSTTQASSAFYQLQYQGRELLFNLTTNPYLMAPGFVSEIRRHSTLGHAHIQTSVPTCHL | |
| | LGDVQDPELEGGFAAISACDGLRGVFQLSNEDYFIEPLDGVSAQPGHAQPHVVYKHQGSR | |
| | KQAQQGDSRPSGTCGMQVPPDLEQQREHWEQQQQKRRQQRSVSKEKWVETLVVADSKMVE | |
| | YHGQPQVESYVLTIMNMVAGLFHDPSIGNPIHISIVRLIILEDEEKDLKITHHAEETLKN | |
| | FCRWQKNINIKGDDHPQHHDTAILLTRKDLCASMNQPCETLGLSHVSGLCHPQLSCSVSE | |
| | DTGMPLAFTVAHELGHSFGIQHDGTGNDCESIGKRPFIMSPQLLYDRGIPLTWSRCSREY | |
| | ITRFLDRGWGLCLDDRPSKDVIALPSVLPGVLYDVNHQCRLQYGSHSAYCEDMDDVCHTL | |
| | WCSVGTTCHSKLDAAVDGTSCGKNKWCLKGECVPEGFQPEAVDGGWSGWSAWSDCSRSCG | |
| | VGVRSSERQCTQPVPKNRGKYCVGERKRSQLCNLPACPPDRPSFRHTQCSQFDGMLYKGK | |
| | LHKWVPVPNDDNPCELHCRPSNSSNTEKLRDAVVDGTPCYQSRISRDICLNGICKNVGCD | |
| | FVIDSGAEEDRCGVCRGDGSTCQTVSRTFKETEGQGYVDIGLIPAGAREILIEEVAEAAN | |
| | FLALRSEDPDKYFLNGGWTIQWNGDYRVAGTTFTYARKGNWENLTSPGPTSEPVWIQLLF | |
| | QEKNPGVHYQYTIQRDSHDQVRPPEFSWHYGPWSKCTVTCGTGVQRQSLYCMERQAGVVA | |
| | EEYCNTLNRPDERQRKCSEEPCPPRWWAGEWQPCSRSCGPEGLSRRAVFCIRSMGLDEQR | |
| | ALELSACEHLPRPLAETPCNRHVICPSTWGVGNWSQCSVTCGAGIRQRSVLCINNTDVPC | |
| | DEAERPITETFCFLQPCQYPMYIVDTGASGSGSSSPELFNEVDFIPNQLAPRPSPASSPK | |
| | PVSISNAIDEEELDPPGPVFVDDFYYDYNFINFHEDLSYGSFEEPHPDLVDNGGWTAPPH | |
| | IRPTESPSDTPVPTAGALGAEAEDIQGSWSPSPLLSEASYSPPGLEQTSINPLANFLTEE | |
| | DTPMGAPELGFPSLPWPPASVDDMMTPVGPGNPDELLVKEDEQSPPSTPWSDRNKLSTDG | |
| | NPLGHTSPALPQSPIPTQPSPPSISPTQASPSPDVVEVSTGWNAAWDPVLEADLKPGHGE | |
| | LPSTVEVASPPLLPMATVPGIWGRDSPLEPGTPTFSSPELSSQHLKTLTMPGTLLLTVPT | |
| | DLRSPGPSGQPQTPNLEGTQSPGLLPTPARETQTNSSKDPEVQPLQPSLEEDGDPADPLP | |
| | ARNASWQVGNWSQCSTTCGLGAIWRLVSCSSGNDEDCTLASRPQPARHCHLRPCAAWRTG | |
| | NWSKCSRNCGGGSSTRDVQCVDTRDLRPLRPFHCQPGPTKPPNRQLCGTQPCLPWYTSSW | |
| | RECSEACGGGEQQRLVTCPEPGLCEESLRPNNSRPCNTHPCTQWVVGPWGQCSAPCGGGV | |
| | QRRLVRCVNTQTGLAEEDSDLCSHEAWPESSRPCATEDCELVEPPRCERDRLSFNFCETL | |
| | RLLGRCQLPTIRAQCCRSCPPLSRGVPSRGHQRVARR | |
| 24 | Human ADAMTS-12 from P58397 | PRT |
| | MPCAQRSWLANLSVVAQLLNFGALCYGRQPQPGPVRFPDRRQEHFIKGLPEYHVVGPVRV | |
| | DASGHFLSYGLHYPITSSRRKRDLDGSEDWVYYRISHEEKDLFFNLTVNQGFLSNSYIME | |
| | KRYGNLSHVKMMASSAPLCHLSGTVLQQGTRVGTAALSACHGLTGFFQLPHGDFFIEPVK | |
| | KHPLVEGGYHPHIVYRRQKVPETKEPTCGLKDSVNISQKQELWREKWERHNLPSRSLSRR | |
| | SISKERWVETLVVADTKMIEYHGSENVESYILTIMNMVTGLFHNPSIGNAIHIVVVRLIL | |
| | LEEEEQGLKIVHHAEKTLSSFCKWQKSINPKSDLNPVHHDVAVLLTRKDICAGFNRPCET | |
| | LGLSHLSGMCQPHRSCNINEDSGLPLAFTIAHELGHSFGIQHDGKENDCEPVGRHPYIMS | |
| | RQLQYDPTPLTWSKCSEEYITRFLDRGWGFCLDDIPKKKGLKSKVIAPGVIYDVHHQCQL | |
| | QYGPNATFCQEVENVCQTLWCSVKGFCRSKLDAAADGTQCGEKKWCMAGKCITVGKKPES | |
| | IPGGWGRWSPWSHCSRTCGAGVQSAERLCNNPEPKFGGKYCTGERKRYRLCNVHPCRSEA | |
| | PTFRQMQCSEFDTVPYKNELYHWFPIFNPAHPCELYCRPIDGQFSEKMLDAVIDGTPCFE | |
| | GGNSRNVCINGICKMVGCDYEIDSNATEDRCGVCLGDGSSCQTVRKMFKQKEGSGYVDIG | |
| | LIPKGARDIRVMEIEGAGNFLAIRSEDPEKYYLNGGFIIQWNGNYKLAGTVFQYDRKGDL | |
| | EKLMATGPTNESVWIQLLFQVTNPGIKYEYTIQKDGLDNDVEQQMYFWQYGHWTECSVTC | |
| | GTGIRRQTAHCIKKGRGMVKATFCDPETQPNGRQKKCHEKACPPRWWAGEWEACSATCGP | |
| | HGEKKRTVLCIQTMVSDEQALPPTDCQHLLKPKTLLSCNRDILCPSDWTVGNWSECSVSC | |
| | GGGVRIRSVTCAKNHDEPCDVTRKPNSRALCGLQQCPSSRRVLKPNKGTISNGKNPPTLK | |
| | PVPPPTSRPRMLTTPTGPESMSTSTPAISSPSPTTASKEGDLGGKQWQDSSTQPELSSRY | |
| | LISTGSTSQPILTSQSLSIQPSEENVSSSDTGPTSEGGLVATTTSGSGLSSSRNPITWPV | |
| | TPFYNTLTKGPEMEIHSGSGEEREQPEDKDESNPVIWTKIRVPGNDAPVESTEMPLAPPL | |
| | TPDLSRESWWPPFSTVMEGLLPSQRPTTSETGTPRVEGMVTEKPANTLLPLGGDHQPEPS | |
| | GKTANRNHLKLPNNMNQTKSSEPVLTEEDATSLITEGFLLNASNYKQLTNGHGSAHWIVG | |
| | NWSECSTTCGLGAYWRRVECSTQMDSDCAAIQRPDPAKRCHLRPCAGWKVGNWSKCSRNC | |
| | SGGFKIREIQCVDSRDHRNLRPFHCQFLAGIPPPLSMSCNPEPCEAWQVEPWSQCSRSCG | |
| | GGVQERGVFCPGGLCDWTKRPTSTMSCNEHLCCHWATGNWDLCSTSCGGGFQKRTVQCVP | |
| | SEGNKTEDQDQCLCDHKPRPPEFKKCNQQACKKSADLLCTKDKLSASFCQTLKAMKKCSV | |
| | PTVRAECCFSCPQTHITHTQRQRRQRLLQKSKEL | |
| 25 | Rat ADAMTS-12 from D3ZTJ3 | PRT |
| | MPCAQGNWMAKLSMVAQLLNFGAFCHGRQAQPWPVRFPDPKQEHFIKSLPEYHIVSPVQV | |
| | DASGHFLSYGLHHPVTGSRKKRAAGGSGDQVYYRISHEEKNLFFNLTVNWEFLSNGYVVE | |
| | RRYGNLSHVKMAASSGQPCHLRGTVLQQGPTIRMGTAALSACQGLTGFFHLPHGDFFIEP | |
| | VKKHPLTEEGYQPHVIYRRQSYRVPETKEPTCGLKDSLDNSVKQELQREKWERKNWPSRS | |
| | LSRRSISKERWVETLVVADTKMVEYHGSENVESYILTIMNMVTGLFHNPSIGNAVHIVVV | |
| | RLILLEEEEQGLKIVHHAEKTLSSFCKWQKSINPKSDLNPVHHDVAVLITRKDICAGVNR | |
| | PCETLGLSQLSGMCQPHRSCNINEDSGLPLAFTIAHELGHSFGIQHDGKENDCEPVGRHP | |
| | YIMSQQIQYDPTPLTWSKCSKEYITRFLDRGRGFCLDDVPRKKGLKSNVIAPGVIYDVHH | |
| | QCQLQYGPNATFCQEVENVCQTLWCSVKGFCRSKLDAAADGTRCGEKKWCMAGKCITVGK | |
| | KPESIPGGWGRWSPWSHCSRTCGAGAQSAERLCNNPEPKFGGKYCTGERKRYRLCNVHPC | |
| | RSDTPTFRQMQCSEFDTVPYKNQFYRWFPVFNPAHPCELYCRPIDEQFSERMLEAVIDGT | |
| | PCFEGGNSRNVCINGICKRVGCDYEIDSNATEDRCGVCLGDGSACQTVKKVFRQKEGSGY | |
| | IDIGLIPKGARDIRVMEIKAAGNFLAIRSEDPEKYYLNGGFIIQWNGNYKLAGTVFQYDR | |
| | KGDLERLMAPGPTNESVWLQLLFQVTNPGIKYEYTVRKDGLDNDVEKLLYFWQFGRWTEC | |
| | SVTCGTGIRRQTAHCVKKGHGIVKTTFCNPETQPSVRQKKCYEKDCPPRWWAGEWEACSM | |
| | TCGPYGEKKRTVLCIQTMGSDEQALPATDCQHLLKPKTLVSCNRDILCPSDWTVGNWSEC | |
| | SVSCGGGVRIRSVTCAKNLNEPCDKTRKPNSRALCGLQQCPFSRRVLKPNKDTVPSGKNP | |
| | TTSEHDHFKPIPASTSRPTPLSTPTVPESVSTSTPTINSLGSTITSQEEPDGIGWQNNST | |
| | QAEEDSHIPTSVGSTSQTPLTSWSWSMQPDDENVSSSAIGPTSESDFWATTSDSGLSSSN | |
| | AMTWQVTPFYSTATTEPEVEIHSGSGEDSDQPLNKEENNSVLWNKIRVPERDAPMEMDAE | |
| | IPLGPPPTSYVTEESSWPPFSTMMKSSLPAWSFKNETPRDEGMITEKSGNIPLPLGGDHQ | |
| | TTSPEKLGNNDQLASANSTNPTQGSGPVLTEEDASTLIEEGFLLNASNYKHLMKDHSPAH | |
| | WIVGNWSKCSTTCGLGAYWRSVECSTGMNADCAAIQRPDPAKKCHLRPCAGWRVGNWSKC | |
| | SRNCSGGFKIREVQCMDGVDHHRSLRPFHCQFLAGVPPPLSMSCNLEPCEEWKVEPWSQC | |
| | SRSCGGGVQERGVFCPGGLCDWTKRPASTVPCNRHLCCHWATGNWELCTTSCGGGSQKRT | |
| | VHCIPSENSTTEDQDQCFCDHQARPPEFQNCNQQACRKSADLTCTKDRLSTSFCQTLKSM | |
| | KKCSVPSVRVQCCLSCPQTQSIHTQRQRKQQMLQNHDTL | |
| 26 | Mouse ADAMTS-12 from Q811B3 | PRT |
| | MPCARGSWLAKLSIVAQLINFGAFCHGRQTQPWPVRFPDPRQEHFIKSLPEYHIVSPVQV | |
| | DAGGHVLSYGLHHPVTSSRKKRAAGGSGDQLYYRISHEEKDLFFNLTVNWEFLSNGYVVE | |
| | KRYGNLSHVKMVASSGQPCHLRGTVLQQGTTVGIGTAALSACQGLTGFFHLPHGDFFIEP | |
| | VKKHPLTEEGSYPHVVYRRQSIRAPETKEPICGLKDSLDNSVKQELQREKWERKTLRSRS | |
| | LSRRSISKERWVETLVVADTKTVEYHGSENVESYILTIMNMVTGLFHSPSIGNLVHIVVV | |
| | RLILLEEEEQGLKIVHHAEKTLSSFCKWQKSINPKSDLNPVHHDVAVLITRKDICAGVNR | |
| | PCETLGLSQLSGMCQPHRSCNINEDSGLPLAFTIAHELGHSFGIQHDGKENDCEPVGRHP | |
| | YIMSQQIQYDPTPLTWSKCSKEYITRFLDRGRGFCLDDIPSKKGLKSNVIAPGVIYDVHH | |
| | QCQLQYGPNATFCQEVENVCQTLWCSVKGFCRSKLDAAADGTRCGEKKWCMAGKCITVGK | |
| | KPESIPGGWGRWSPWSHCSRTCGAGAQSAERLCNNPEPKFGGKYCTGERKRYRLCNVHPC | |
| | RSDTPTFRQMQCSEFDTVPYKNQFYRWFPVFNAAHPCELYCRPIDEQFSERMLEAVIDGT | |
| | PCFEGGNSRNVCINGICKRVGCDYEIDSNATEDRCGVCLGDGSACQTVKKLFRQKEGSGY | |
| | VDIGLIPKGARDIRVMEIKAAGNFLAIRSEDPEKYYLNGGFIIQWNGNYKLAGTVFQYDR | |
| | KGDLEKLIAPGPTNESVWLQLLFQVTNPGIKYEYTVRKDGLDNDVEKLLYFWQFGRWTEC | |
| | SVTCGTGIRRQAAHCVKKGHGIVKTTFCNPETQPSVRQKKCHEKDCPPRWWAGEWEACST | |
| | TCGPYGEKKRTVLCIQTMGSDEQALPATDCQHLLKPKALVSCNRDILCPSDWTVGNWSEC | |
| | SVSCGGGVRIRSVTCAKNLNEPCDKTRKPNSRALCGLQQCPFSRRVLKPNKDIAPSGKNQ | |
| | STAEHDPFKPIPAPTSRPTPLSTPTVPESMSTSTPTINSLGSTIASQEDANGMGWQNNST | |
| | QAEEGSHFPTSSGSTSQVPVTSWSLSIQPDDENVSSSAIGPTSEGDFWATTTSDSGLSSS | |
| | DAMTWQVTPFYSTMTTDPEVEIHSGSGEDSDQPLNKDKSNSVIWNKIGVPEHDAPMETDA | |
| | ELPLGPPPTSYMGEEPSWPPFSTKMEGSLPAWSFKNETPRDDGMIAEKSRKIPLPLAGDH | |
| | HPATSEKLENHDKLALPNTTNPTQGFGPVLTEEDASNLIAEGFLLNASDYKHLMKDHSPA | |
| | YWIVGNWSKCSTTCGLGAYWRSVECSSGVDADCTTIQRPDPAKKCHLRPCAGWRVGNWSK | |
| | CSRNCSGGFKIREVQCMDSLDHHRSLRPFHCQFLAGAPPPLSMSCNLEPCGEWQVEPWSQ | |
| | CSRSCGGGVQERGVSCPGGLCDWTKRPATTVPCNRHLCCHWATGNWELCNTSCGGGSQKR | |
| | TIHCIPSENSTTEDQDQCLCDHQVKPPEFQTCNQQACRKSADLTCLKDRLSISFCQTLKS | |
| | MRKCSVPSVRAQCCLSCPQAPSIHTQRQRKQQLLQNHDML | |

### Definitions

Unless otherwise defined, all scientific and technical terms used in the description, figures and claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and not intended to be limiting. Unless stated otherwise, the following terms used in this document, including the description and claims, have the definitions given below.

The terms “comprising”, “including”, “containing”, “having” etc. shall be read expansively or open-ended and without limitation.

Singular forms such as “a”, “an” or “the” include plural references unless the context clearly indicates otherwise.

Unless otherwise indicated, the term “at least” preceding a series of elements is to be understood to refer to every element in the series. The terms “at least one” and “at least one of” include for example, one, two, three, four, or five or more elements.

The term “ADAMTS-7” (also ADAMTS7, ADAM-TS 7, ADAM-TS7) refers to the protein A disintegrin and metalloproteinase with thrombospondin motifs 7. The ADAMTS-7 protein is encoded by the gene ADAMTS-7. The ADAMTS-7 protein comprises human, murine, rat and further mammalian and non-mammalian homologues. Sequence(s) for human ADAMTS-7 are accessible via UniProt Identifier Q9UKP4 (ATS7_HUMAN), for instance human isoform Q9UKP4-1. Sequence(s) for murine ADAMTS-7 are accessible via UniProt Identifier Q68SA9 (ATS7 MOUSE). Different isoforms, variants and SNPs may exist for the different species and are all comprised by the term ADAMTS-7. Also comprised are ADAMTS-7 molecules before and after maturation—independently from cleavage of one or more pro-domains. In addition, synthetic variants of the ADAMTS-7 protein may be generated and are comprised by the term ADAMTS-7. The protein ADAMTS-7 may furthermore be subject to various modifications, e.g, synthetic or naturally occurring modifications. Recombinant functional human ADAMTS-7 (e.g. according to SEQ ID NO: 1 and 2) can be manufactured as described in the Examples.

The term “ADAMTS-12” (also ADAMTS12, ADAM-TS 12, ADAM-TS12) refers to the protein A disintegrin and metalloproteinase with thrombospondin motifs 12. Such proteins preferably include a ADAMTS-12 catalytic domain. The ADAMTS-12 protein is encoded by the gene ADAMTS-12. The ADAMTS-12 protein comprises human, murine, rat and further mammalian and non-mammalian homologues. Sequence(s) for human ADAMTS-12 including the catalytic domains are accessible via UniProt Identifier P58397 (ATS12_HUMAN), for instance human isoform P58397-1. Sequence(s) for murine ADAMTS-12 are accessible via UniProt Identifier Q811B3 (ATS12 MOUSE). Different isoforms and variants may exist for the different species and are all comprised by the term ADAMTS-12. Also comprised are ADAMTS-12 molecules before and after maturation independently from cleavage of one or more pro-domains. In addition, synthetic variants of the ADAMTS-12 protein may be generated and are comprised by the term ADAMTS-12. The protein ADAMTS-12 may furthermore be subject to various modifications, e.g., synthetic or naturally occurring modifications. Recombinant functional human ADAMTS-12 (e.g., according to SEQ ID NO: 15) can be manufactured as described in the Examples.

The terms “ADAMTS-4” and “ADAMTS-5” refer to the protein A disintegrin and metalloproteinase with thrombospondin motifs 4 and 5, respectively. The ADAMTS-4 and -5 proteins are encoded by the genes ADAMTS4 and ADAMTS-5, respectively. These proteins comprise human, murine, rat and further mammalian and non-mammalian homologues. Sequence(s) for human ADAMTS-4/-5 are accessible via UniProt Identifier 075173 (ATS4_HUMAN)/Q9UNA0 (ATS5_HUMAN), respectively. Different isoforms and variants may exist. Recombinant active human ADAMTS-4 and ADAMTS-5 can be manufactured as known in the art.

The terms “MMP2”, “MMP12”, and “MMP15” refer to the 72 kDa type IV collagenase, Macrophage metalloelastase 2 and 12 and Matrix metalloproteinase-15, respectively. The MMP2, MMP12, and MMP15 proteins are encoded by the genes MMP2, MMP12, and MMP15, respectively. The proteins comprise human, murine, rat and further mammalian and non-mammalian homologues. Sequence(s) for human MMP2, MMP12, and MMP15 are accessible via UniProt Identifier P08253 (MMP2_HUMAN), P39900 (MMP12_HUMAN), and P51511 (MMP15_HUMAN), respectively. Different isoforms and variants may exist. Recombinant active human ADAMTS-4 and ADAMTS-5 can be manufactured as known in the art.

The term “ADAM17” refers to Disintegrin and metalloproteinase domain-containing protein 17, encoded by the gene ADAM17. The protein comprises human, murine, rat and further mammalian and non-mammalian homologues. Sequence(s) for human ADAM17 are accessible via UniProt Identifier P78536 (ADA17_HUMAN). Different isoforms and variants may exist. Recombinant active human ADAM17 can be manufactured as known in the art.

The term “prodomain” includes parts of ADAMTS-7 or ADAMTS-12 that are relatively N-terminal to the respective protein's biologically functional chain (e.g., parts having metalloprotease function and disintergrin motifs). For example, a prodomain of ADAMTS-7 as provided in SEQ ID NO: 1 can include its signal peptide (residues 1-20) and its propeptide (residues 21-217), both of which are N-terminal to its catalytic domain, although not necessarily immediately N-terminal to it. In some embodiments, prodomain of ADAMTS-7 or ADAMTS-12 includes 75%, 80%, 85%, 90%, 95%, or 100% of the N-terminal part of the respective protein that has its signal peptide plus its propeptide. The term “prodomain” also encompasses the parts of the encoded polypeptide that are processed (e.g., cleaved off) before generation of the functional enzymatic chain in the natural environment of the enzyme.

The term “CD domain” includes parts of ADAMTS-7 or ADAMTS-12 that have ADAMTS-7 or ADAMTS-12 functionality, respectively, and that are C-terminal to the respective protein's prodomain. In some embodiments, the term “CD domain” refers to the catalytic domain plus disintegrin part of the respective protein (e.g., as characterized by UniProt, which might use the alternative term “peptidase” for the catalytic domain), potentially also including any residues C-terminal to the respective protein's prodomain and N-terminal to the respective protein's peptidase domain. In some embodiments, the CD domain includes 75%, 80%, 85%, 90%, 95%, or 100% of the part of the respective enzyme that has its disintegrin domain, its peptidase domain, and any residues it might have between its prodomain and its peptidase domain.

The term “functional protein” refers to a protein that has biological activity. For example, functional ADAMTS-7 refers to ADAMTS-7 that is able to catalyze the proteolytic cleavage of its (natural) substrate(s), e.g. TSP1 and/or COMP. Similarly, a functional segment of a protein or domain (e.g., CD domain, catalytic domain) has biological activity. In the specific context of a prodomain, a functional segment of a prodomain has activity in the sense that it facilitates expressing or purifying the biologically active domain it is associated with (e.g., by improving the folding of the CD domain or catalytic domain).

The term “metalloproteinase” refers to a protease enzyme whose catalytic mechanism involves a metal. Therefore, a functional metalloproteinase is a functional protein, wherein the protein is a protease and wherein the protease is a metalloproteinase according to the foregoing definitions.

The expression “a cleavage site for a protease” refers to any peptide or protein sequence that is recognized and cleaved by the functional protease. A cleavage site for ADAMTS-7 thus refers to any peptide or protein sequence that is recognized and cleaved by functional ADAMTS-7. For example, being natural substrates of ADAMTS-7, the sequences of proteins COMP and TSP1 both comprise cleavage sites for ADAMTS-7. In particular the subsequence DELSSMVLELRGLRT (derived from TSP1, residues 275-289; provided as residues 1-15 of SEQ ID NO: 4) constitutes or comprises a cleavage site for ADAMTS-7 and ADAMTS-12.

A “substrate” is a molecule upon which an enzyme acts. For example, the substrate of a proteinase can be a peptide or protein or derivative thereof, which is cleaved by the proteinase. Metalloproteinase paralogs ADAMTS-7/-12 on the one hand and their common peptide substrate on the other hand are interrelated in the sense of a plug and socket relationship.

The term “COMP”, TSP-5 or TSP5 refers to the protein Cartilage oligomeric matrix protein. The COMP protein is encoded by the gene COMP. The COMP protein comprises human, murine, rat and further mammalian and homologues. Sequence(s) for human COMP are accessible via UniProt Identifier P49747 (COMP_HUMAN), for instance human isoform P49747-1. Sequence(s) for murine COMP are accessible via UniProt Identifier Q9ROG6 (COMP_MOUSE). Different isoforms and variants may exist for the different species and are all comprised by the term COMP. Also comprised are COMP molecules before and after maturation, independently from cleavage of one or more pro-domains. In addition, synthetic variants of the COMP protein may be generated and are comprised by the term COMP. The protein COMP may furthermore be subject to various modifications, e.g, synthetic or naturally occurring modifications. Recombinant human COMP or derivatives thereof can be manufactured as described in the Examples.

The term “TSP1” (also THBS1 or TSP) refers to the protein Thrombospondin-1. The TSP1 protein is encoded by the gene THBS1. The TSP1 protein comprises human, murine, rat and further mammalian and non-mammalian homologues. Sequence(s) for human TSP1 are accessible via UniProt Identifier P07996 (TSP1_HUMAN), for instance human isoform P07996-1. Sequence(s) for murine TSP1 are accessible via UniProt Identifier P35441 (TSP1_MOUSE). Different isoforms and variants may exist for the different species and are all comprised by the term TSP1. Also comprised are TSP1 molecules before and after maturation, independently from cleavage of one or more pro-domains. In addition, synthetic variants of the TSP1 protein may be generated and are comprised by the term TSP1. The protein TSP1 may furthermore be subject to various modifications, e.g, synthetic or naturally occurring modifications. Recombinant human TSP1 or derivatives thereof can be manufactured as described in the Examples.

The term “fluorophore” as used herein refers to a molecule or chemical group that has the ability to absorb energy from light, transfer this energy internally, and emit this energy as light of a characteristic wavelength. Typically, since some energy is lost during this process, the energy of the emitted fluorescence light is lower than the energy of the absorbed light, and therefore emission occurs at a longer wavelength than absorption. A variety of fluorophores and quenchers has been described in the art and can be used in the methods and compositions provided herein, see for example Bajar et al, Sensors 2016, A Guide to Fluorescent Protein FRET Pairs.

The term “quencher” as used herein refers to a molecule or chemical group that has the ability to decrease the fluorescence intensity of a given fluorophore. A variety of processes can result in quenching, such as excited state reactions, energy transfer, complex-formation and collisional quenching. Dark quenchers are dyes with no native fluorescence. Quencher fluorescence can increase background noise due to overlap between the quencher and reporter fluorescence spectra. A variety of fluorophores and quenchers has been described in the art and can be used in the methods and compositions provided herein, see for example Bajar et al, Sensors 2016, A Guide to Fluorescent Protein FRET Pairs. In certain embodiments, suitable quenchers include, for example, DDQ-I A (430 nm), Dabcyl (475 nm), Eclipse B (530 nm), Iowa Black FQ C (532 nm), BHQ-1D (534 nm), QSY-7 E (571 nm), BHQ-2 D (580 nm), DDQ-II A (630 nm), Iowa Black RQ C (645 nm), QSY-21 E (660 nm) or BHQ-3 D (670 nm). Examples of quenchers that can be used in certain of the methods and compositions provided herein include Dabsyl (dimethylaminoazobenzene sulfonic acid), which absorbs in the green spectrum and is often used with fluorescein, black hole quenchers which are capable of quenching across the entire visible spectrum, Qxl quenchers which span the full visible spectrum, Iowa black FQ (absorbs in the green-yellow part of the spectrum), Iowa black RQ (blocks in the orange-red part of the spectrum) and IRDye QC-1 (quenches dyes from the visible to the near-infrared range (500-900 nm)).

Suitable internally quenched fluorescent (IQF)/FRET pairs include ABz-Tyr(NO2), ABz-EDDNP, Trp-Dansyl, and 7-methoxy-coumarin-4-yl acetic acid-2,4-dinitrophenyl-lysine (MCA-Lys(DNP)) (Poreba, Marcin et al., Highly sensitive and adaptable fluorescence-quenched pair discloses the substrate specificity profiles in diverse protease families. Scientific reports 7, 43135. (2017), doi:10.1038/srep43135). Further suitable examples are listed in: Poreba M. & Drag M. Current strategies for probing substrate specificity of proteases. Curr Med Chem 17, 3968-3995 (2010).

The term “construct” refers to nucleic acids (e.g., double stranded DNA, which can be in the form of a plasmid).

The term “align” in the context of two sequences (e.g., amino acid sequences), includes arranging the two sequences with respect to each other (e.g., the first sequence along a first row, and the second sequence along a second row, potentially with gap(s) in one or both sequences) to obtain a measure of their relationship to each other (e.g., % identity, % similarity, alignment score). A particular alignment of two sequences can be optimal (i.e., there are no alignments of the two sequences that result in a higher alignment score, if alignment score is the metric of concern for optimality) or non-optimal. In addition, a particular alignment can be global or local with respect to either sequence. For example, if the alignment is global with respect to both sequences (i.e., a global-global alignment), then all residues of both of the sequences are factored in to the calculation of the measure of their relationship, including any internal or external gaps in either sequence. In contrast, in a global-local alignment, while all residues and gaps in the first sequence are considered, no external gaps in the second sequence are considered, thereby allowing fitting the first sequence into a part of the second sequence.

The term “Needleman-Wunsch score” implies that either a global-global or a global-local (or local-global) alignment has been used to generate the alignment score. When partiality modifiers are used for both the first sequence (e.g., “portion”) and the second sequence (e.g., “segment”), this implies that the alignment is global-global with respect to the specified parts. A “Needleman-Wunsch score” includes scores calculated by the originally published method (S. B. Needleman & C. D. Wunsch, A general method applicable to the search for similarities in the amino acid sequence of two proteins, J. Mol. Biol. 48(3):443-53 (1970)) as well as by subsequent refinements of the method. Although Needleman-Wunsch algorithm is able to, and is designed to, find an optimal alignment, and thereby a maximum alignment score, the term “Needleman-Wunsch score” as used here is not restricted to the optimal/maximum score; it can be the alignment score of any alignment of the two sequences as long as at least one of the sequences (e.g., as defined, for example as a residue range) is considered globally.

Alignment methods (e.g., Needleman-Wunsch) that generate an alignment score (e.g., Needleman-Wunsch score) can make use of a substitution matrix. A particular substitution matrix that can be used is BLOSUM62, which is reproduced below in Table 2.

TABLE 2

| | A | R | N | D | C | Q | E | G | H | I | L | K | M | F | P | S | T | W | Y | V | B | Z | X | * |
|---|---|---|---|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 | −2 | −1 | 0 | −4 |
| R | −1 | 5 | 0 | −2 | −3 | 1 | 0 | −2 | 0 | −3 | −2 | 2 | −1 | −3 | −2 | −1 | −1 | −3 | −2 | −3 | −1 | 0 | −1 | −4 |
| N | −2 | 0 | 6 | 1 | −3 | 0 | 0 | 0 | 1 | −3 | −3 | 0 | −2 | −3 | −2 | 1 | 0 | −4 | −2 | −3 | 3 | 0 | −1 | −4 |
| D | −2 | −2 | 1 | 6 | −3 | 0 | 2 | −1 | −1 | −3 | −4 | −1 | −3 | −3 | −1 | 0 | −1 | −4 | −3 | −3 | 4 | 1 | −1 | −4 |
| C | 0 | −3 | −3 | −3 | 9 | −3 | −4 | −3 | −3 | −1 | −1 | −3 | −1 | −2 | −3 | −1 | −1 | −2 | −2 | −1 | −3 | −3 | −2 | −4 |
| Q | −1 | 1 | 0 | 0 | −3 | 5 | 2 | −2 | 0 | −3 | −2 | 1 | 0 | −3 | −1 | 0 | −1 | −2 | −1 | −2 | 0 | 3 | −1 | −4 |
| E | −1 | 0 | 0 | 2 | −4 | 2 | 5 | −2 | 0 | −3 | −3 | 1 | −2 | −3 | −1 | 0 | −1 | −3 | −2 | −2 | 1 | 4 | −1 | −4 |
| G | 0 | −2 | 0 | −1 | −3 | −2 | −2 | 6 | −2 | −4 | −4 | −2 | −3 | −3 | −2 | 0 | −2 | −2 | −3 | −3 | −1 | −2 | −1 | −4 |
| H | −2 | 0 | 1 | −1 | −3 | 0 | 0 | −2 | 8 | −3 | −3 | −1 | −2 | −1 | −2 | −1 | −2 | −2 | 2 | −3 | 0 | 0 | −1 | −4 |
| I | −1 | −3 | −3 | −3 | −1 | −3 | −3 | −4 | −3 | 4 | 2 | −3 | 1 | 0 | −3 | −2 | −1 | −3 | −1 | 3 | −3 | −3 | −1 | −4 |
| L | −1 | −2 | −3 | −4 | −1 | −2 | −3 | −4 | −3 | 2 | 4 | −2 | 2 | 0 | −3 | −2 | −1 | −2 | −1 | 1 | −4 | −3 | −1 | −4 |
| K | −1 | 2 | 0 | −1 | −3 | 1 | 1 | −2 | −1 | −3 | −2 | 5 | −1 | −3 | −1 | 0 | −1 | −3 | −2 | −2 | 0 | 1 | −1 | −4 |
| M | −1 | −1 | −2 | −3 | −1 | 0 | −2 | −3 | −2 | 1 | 2 | −1 | 5 | 0 | −2 | −1 | −1 | −1 | −1 | 1 | −3 | −1 | −1 | −4 |
| F | −2 | −3 | −3 | −3 | −2 | −3 | −3 | −3 | −1 | 0 | 0 | −3 | 0 | 6 | −4 | −2 | −2 | 1 | 3 | −1 | −3 | −3 | −1 | −4 |
| P | −1 | −2 | −2 | −1 | −3 | −1 | −1 | −2 | −2 | −3 | −3 | −1 | −2 | −4 | 7 | −1 | −1 | −4 | −3 | −2 | −2 | −1 | −2 | −4 |
| S | 1 | −1 | 1 | 0 | −1 | 0 | 0 | 0 | −1 | −2 | −2 | 0 | −1 | −2 | −1 | 4 | 1 | −3 | −2 | −2 | 0 | 0 | 0 | −4 |
| T | 0 | −1 | 0 | −1 | −1 | −1 | −1 | −2 | −2 | −1 | −1 | −1 | −1 | −2 | −1 | 1 | 5 | −2 | −2 | 0 | −1 | −1 | 0 | −4 |
| W | −3 | −3 | −4 | −4 | −2 | −2 | −3 | −2 | −2 | −3 | −2 | −3 | −1 | 1 | −4 | −3 | −2 | 11 | 2 | −3 | −4 | −3 | −2 | −4 |
| Y | −2 | −2 | −2 | −3 | −2 | −1 | −2 | −3 | 2 | −1 | −1 | −2 | −1 | 3 | −3 | −2 | −2 | 2 | 7 | −1 | −3 | −2 | −1 | −4 |
| V | 0 | −3 | −3 | −3 | −1 | −2 | −2 | −3 | −3 | 3 | 1 | −2 | 1 | −1 | −2 | −2 | 0 | −3 | −1 | 4 | −3 | −2 | −1 | −4 |
| B | −2 | −1 | 3 | 4 | −3 | 0 | 1 | −1 | 0 | −3 | −4 | 0 | −3 | −3 | −2 | 0 | −1 | −4 | −3 | −3 | 4 | 1 | −1 | −4 |
| Z | −1 | 0 | 0 | 1 | −3 | 3 | 4 | −2 | 0 | −3 | −3 | 1 | −1 | −3 | −1 | 0 | −1 | −3 | −2 | −2 | 1 | 4 | −1 | −4 |
| X | 0 | −1 | −1 | −1 | −2 | −1 | −1 | −1 | −1 | −1 | −1 | −1 | −1 | −1 | −2 | 0 | 0 | −2 | −1 | −1 | −1 | −1 | −1 | −4 |
| * | −4 | −4 | −4 | −4 | −4 | −4 | −4 | −4 | −4 | −4 | −4 | −4 | −4 | −4 | −4 | −4 | −4 | −4 | −4 | −4 | −4 | −4 | −4 | 1 |

In this table, the first 20 columns (and the first 20 rows) labelled with single-letter amino acid codes represent the standard amino acids. The remaining columns/rows represent additional residue types (e.g., “B” for Asx (Asn/Asp), “Z” for Glx (Gln/Glu), “X” for any amino acid, and “*” for a translation stop encoded by a termination codon). If the sequences of interest have only a subset of these residues, then a corresponding sub-matrix of BLOSUM62 can also be alternatively sufficient for aligning them (e.g., the scores from the upper-left 20×20 part of the scores if only standard amino acids that are singly-identified are of concern).

As a demonstration of using BLOSUM62 in calculating an alignment score, if residues 1-8 of SEQ ID NO: 14 (“EEVKAKVQ”) were aligned with themselves, the alignment score would be the sum of scores for each of those amino acids pairing with itself (e.g., when residue “A” in the first sequence pairs with itself, an “A,” in the second sequence, it contributes a score of 4, as seen in the cell at row 2, column 2 of the BLOSUM62 matrix). Therefore, the alignment score in this hypothetical example would be 37 (5+5+4+5+4+5+4+5). If the “A” in the second sequence, in this hypothetical, is changed into a “W,” the alignment score would be 30 (5+5+4+5-3+5+4+5). If the “E” at residue one, or the “K” at residue four, or the “Q” at residue eight is deleted in the second sequence instead, in this hypothetical, the alignment score in each case would be 20 (the original total score of 37 is lessened by 5 due to the deleted residue, and there is an additional total gap penalty of 12 in this case, as explained further next). The total gap penalty is calculated by summing a gap extension penalty as multiplied by the number of residues in the gap with a gap-opening penalty. As an example, a gap-opening penalty of 11 and a gap extension penalty of 1 can be used. In that case, a single gap as in the last hypothetical example would result in a total gap penalty of 12 (11+1*1), regardless of whether the gap is internal or external, which can be the case for global-global alignments. If the gap were three amino acids long in the middle of a sequence, the total gap penalty would be 14 (11+1*3), which would be subtracted from of the scores of the aligning residues (identical as well as non-identical) to arrive at the alignment score.

Using a substitution matrix such as BLOSUM62 is superior to cruder methods such as percent identity calculations, at least because different aligned identical residues can give different contributions to the overall score depending on how rare or common themselves or their mutations are (e.g., a W:W alignment contributes 11, as seen in Table 2, while an A:A alignment contributes only 4). In addition, mutations into different residues can also be treated differently with a substitution matrix (e.g., a D:E change has a positive score, 2, as seen in Table 2, while a D:L change has a negative score, −4, whereas each of these changes would be clumped as the same non-identical change in a percent-identity approach). Overall, using a substitution matrix like BLOSUM62 provides a dramatically more sensitive measure for inferring sequence relatedness than percent identity methods, since the matrix allows calibrating the score of changing each of the amino acids (e.g., 20) into each of the amino acids (e.g., 20) individually, while with percent identity the changes are limited to merely identical and non-identical ones. As a result, a polypeptide sequence defined with respect to its alignment to a reference sequence in terms of a Needleman-Wunsch score obtained by using BLOSUM62 matrix is significantly more likely to have structural features, physical properties, and functional features in common with the polypeptide of the reference sequence.

For percent sequence identity values, the same alignment method (e.g., Needleman-Wunsch algorithm for global-global alignment, using BLOSUM62 matrix, with gap opening penalty of 11 and a gap extension penalty of 1) can be used to obtain the alignment, after which the pairs of aligned identical residues can be counted and then divided by the total length of the alignment (including gaps, internal as well as external) to arrive at the percent identity value. For percent similarity values, the same approach as for percent identity values can be used, except that what is counted, instead of pairs of identical residues, would be the aligned residue pairs with BLOSUM62 values that are not negative (i.e., ≥0).

Numerous programs as well as web sites exist for calculation of alignment scores. For example, executables for local use can be downloaded from the UVa FASTA Server (available from World Wide Web at fasta.bioch.virginia.edu/fasta_www2/fasta_list2.shtml). Among the programs available at the UVa FASTA server, ggsearch can perform global-global alignments (e.g., using Needleman-Wunsch algorithm, and BLOSUM62 matrix with gap opening penalty of 11 and a gap extension penalty of 1) and glsearch can perform global-local alignments. Similar functionality is also available through an online submission form at the same server (e.g., World Wide Web at fasta.bioch.virginia.edu/fasta_www2/fasta_www.cgi?rm=select&pgm=gnw, after selecting “Align two sequences” to align two sequences rather than one sequence against a database). Additional online sources for similar functionality include the National Center for Biotechnology Information (available through the World Wide Web at https://blast.ncbi.nlm.nih.gov/Blast.cgi) and the European Bioinformatics Institute of the European Molecular Biology Laboratory (available through the World Wide Web at https://www.ebi.ac.uk/Tools/sss/fasta/). In some embodiments, one can also use BLAST (e.g., by choosing the same score matrix and penalty parameters) to find out whether a given sequence falls within a certain percentage identity or a certain alignment score range with respect to a given reference sequence. Although BLAST need not always result in a global alignment, for highly similar sequences it might be as useful as the other global-global or global-local alignment tools described above.

### EMBODIMENTS

According to a first aspect, provided herein is a recombinant nucleic acid sequence for the improved expression and purification of functional ADAMTS-7.

According to some first embodiments according to the first aspect, there is provided a recombinant nucleic acid for expression of an ADAMTS-7 polypeptide that comprises a rodent prodomain of ADAMTS-7 as a first portion and a functional human ADAMTS-7 (e.g., a functional human ADAMTS-7 CD domain or catalytic domain) as a second portion.

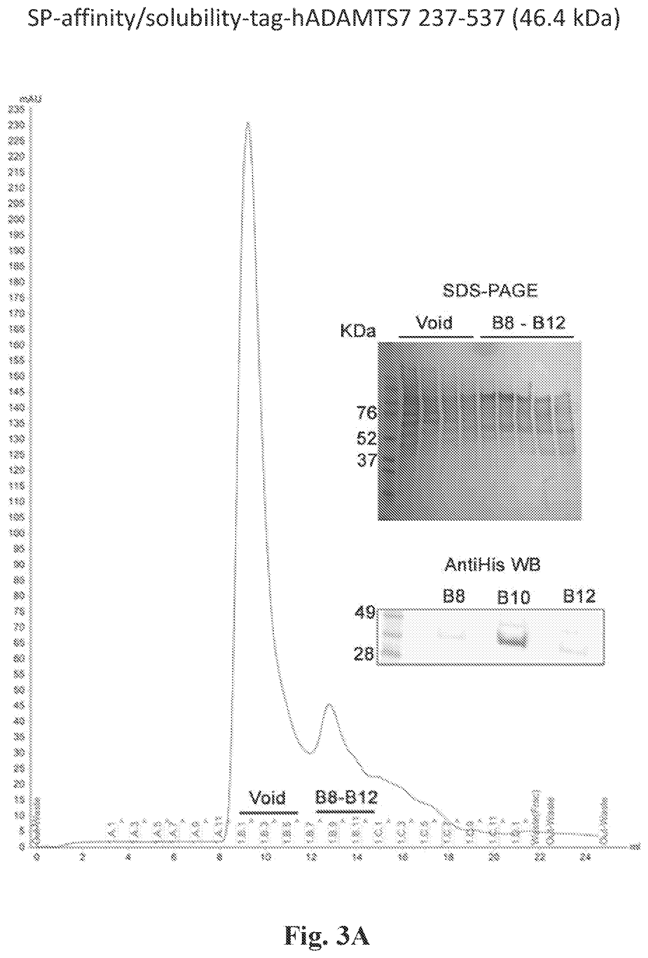

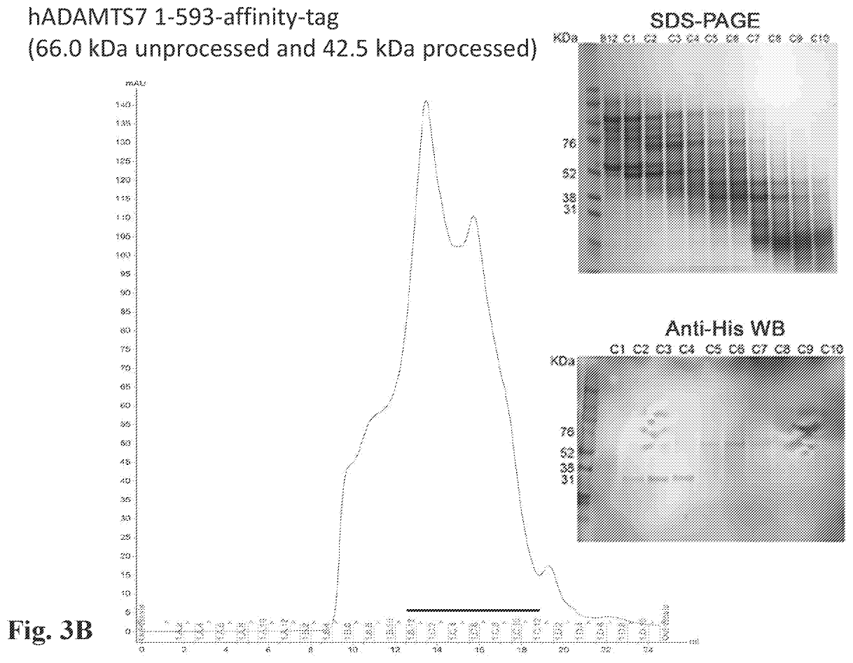

Commercial expression and purification of recombinant functional ADAMTS-7 has so far been a challenge (see, e.g., FIG. 3A and FIG. 3B), rendering the development of an assay for the identification and characterization of modulators of ADAMTS-7 extremely difficult. For example, as shown in FIG. 3A and FIG. 3B and explained in its brief description, expression of hADAMTS-7 (residues 237-537) or hADAMTS-7 Pro-CD-TSR1 (residues 1-593) yielded little soluble proteins. The nucleic acids according to the first aspect solved the problem of protein solubility as well as expression yield, both of which were lower for fully human ADAMTS-7 constructs compared to hybrid constructs (e.g., Example 2).

The constructs according to the first aspect are suited for the production of sufficient amounts of ADAMTS-7 with reproducible activity and purity. The resulting recombinant functional ADAMTS-7 can therefore be used in an assay for the identification and characterization of modulators of ADAMTS-7.

In order to obtain a suitable construct for the expression of functional ADAMTS-7, more than 50 E. coli constructs, 20 constructs from Baculovirus and 30 HEK293 constructs were designed and evaluated to achieve the efficient expression of functional ADAMTS-7. The recombinant nucleic acid according to the first aspect surprisingly solved these problems and enabled the efficient expression and purification of functional ADAMTS-7 protein: In particular it was surprisingly found that a rodent prodomain is more effective in driving folding of the catalytic domain of human ADAMTS-7, thereby improving the yield of the soluble ADAMTS-7 proteins—10 fold (see Example 2).

In some embodiments according to the first aspect, the recombinant nucleic acid sequence encodes for a recombinant polypeptide that comprises a first portion and a second portion, wherein the first portion has a sequence identity of >80% with the sequence of residues 1-217 of SEQ ID NO: 1 or with the sequence of residues 1-217 of SEQ ID NO: 2, and the second portion has a sequence identity of >80% with the sequence of residues 218-518 of SEQ ID NO: 1.

In some embodiments according to the first aspect, the recombinant nucleic acid sequence encodes for a recombinant polypeptide that comprises a first portion and a second portion, wherein the first portion has a sequence identity of >90% with the sequence of residues 1-217 of SEQ ID NO: 1 or with the sequence of residues 1-217 of SEQ ID NO: 2, and the second portion has a sequence identity of >90% with the sequence of residues 218-518 of SEQ ID NO: 1.

In some embodiments according to the first aspect, the recombinant nucleic acid sequence encodes for a recombinant polypeptide that comprises a first portion and a second portion, wherein the first portion has a sequence identity of >95% with the sequence of residues 1-217 of SEQ ID NO: 1 or with the sequence of residues 1-217 of SEQ ID NO: 2, and the second portion has a sequence identity of >95% with the sequence of residues 218-518 of SEQ ID NO: 1.

In some embodiments according to the first aspect, the recombinant nucleic acid sequence encodes for a recombinant polypeptide that comprises a first portion and a second portion, wherein the first portion has a sequence identity of >98% with the sequence of residues 1-217 of SEQ ID NO: 1 or with the sequence of residues 1-217 of SEQ ID NO: 2, and the second portion has a sequence identity of >98% with the sequence of residues 218-518 of SEQ ID NO: 1.

In some embodiments according to the first aspect, the recombinant nucleic acid sequence encodes for a recombinant polypeptide that comprises a first portion and a second portion, wherein the first portion comprises residues 1-217 of SEQ ID NO: 1 or residues 1-217 of SEQ ID NO: 2, and/or the second portion comprises residues 218-518 of SEQ ID NO: 1 (e.g., Example 1). In some embodiments, the second portion is C-terminal to the first portion.

In some embodiments according to the first aspect, the recombinant nucleic acid sequence encodes for a polypeptide that has a first portion and a second portion. The first portion of the polypeptide, in some embodiments, has an amino acid sequence that when aligned with an amino acid sequence of a ADAMTS-7 prodomain or a fragment thereof from a first species generates a Needleman-Wunsch score greater than 700, if BLOSUM62 matrix, a gap opening penalty of 11, and a gap extension penalty of 1 are used. In some embodiments, this generated Needleman-Wunsch score is greater than 750, 800, 850, 900, 950, 1000, 1050, or 1100. In some embodiments, the first portion and the ADAMTS-7 prodomain share a sequence identity that is at least 70%, 75%, 80%, 85%, 90%, 95%, 97% or 98%. The second portion of the polypeptide, in some embodiments, has an amino acid sequence that when aligned with an amino acid sequence of a ADAMTS-7 CD domain or a fragment thereof from a second species generates a Needleman-Wunsch score greater than 1000 if BLOSUM62 matrix, a gap opening penalty of 11, and a gap extension penalty of 1 are used. In some embodiments, this generated Needleman-Wunsch score is greater than 1050, 1100, 1150, 1200, 1250, 1300, 1350, 1400, 1450, 1500, 1550, 1600, or 1650. In some embodiments, the second portion and the ADAMTS-7 CD domain share a sequence identity that is at least 70%, 75%, 80%, 85%, 90%, 95%, 97% or 98%. In some embodiments of these aspects, the second portion has an amino acid sequence that aligns with a functional segment of an ADAMTS-7 catalytic domain amino acid sequence from a second species with a Needleman-Wunsch score greater than 700 when BLOSUM62 matrix, a gap opening penalty of 11, and a gap extension penalty of 1 are used. In some embodiments, this generated Needleman-Wunsch score is greater than 750, 800, 850, 900, 950, 1000, 1050, 1100, or 1150. In some embodiments, the second portion and the ADAMTS-7 catalytic domain share a sequence identity that is at least 70%, 75%, 80%, 85%, 90%, 95%, 97% or 98%.

In some embodiments according to the first aspect, the recombinant nucleic acid sequence encodes for a recombinant polypeptide that comprises a first portion having an amino acid sequence that aligns with an amino acid sequence of an ADAMTS-7 prodomain or a fragment thereof from a first species with a Needleman-Wunsch score greater than 700, when BLOSUM62 matrix, a gap opening penalty of 11, and a gap extension penalty of 1 are used; and a second portion having an amino acid sequence that aligns with an amino acid sequence of a ADAMTS-7 CD domain or a fragment thereof from a second species with a Needleman-Wunsch score greater than 1000 when BLOSUM62 matrix, a gap opening penalty of 11, and a gap extension penalty of 1 are used. In some embodiments of the first aspect, the second portion has an amino acid sequence that aligns with a functional segment of an ADAMTS-7 catalytic domain amino acid sequence from a second species with a Needleman-Wunsch score greater than 700 when BLOSUM62 matrix, a gap opening penalty of 11, and a gap extension penalty of 1 are used.

In some embodiments, the first species is a non-human species, such as a rodent species, such as rat. In some different or the same embodiments, the second species is human, e.g. the CD domain is derived from human ADAMTS-7. In some embodiments, the first species is a rodent species, and the second species is human.

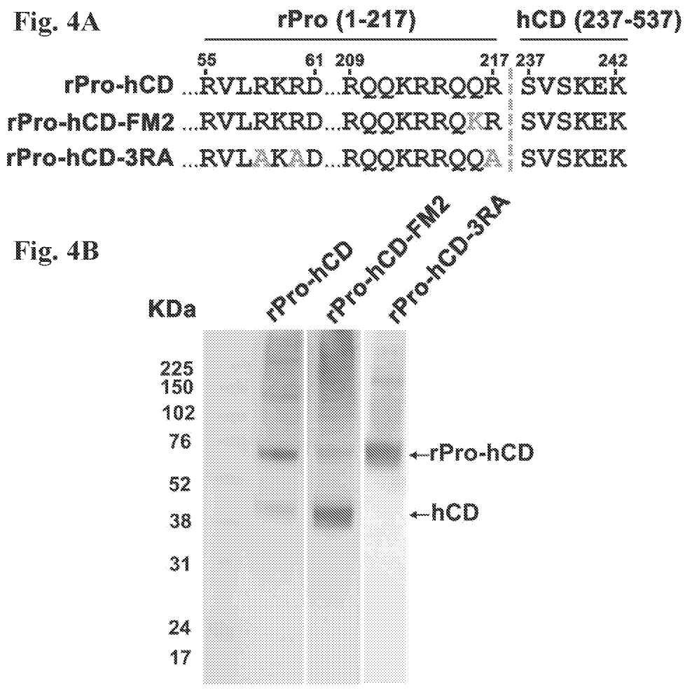

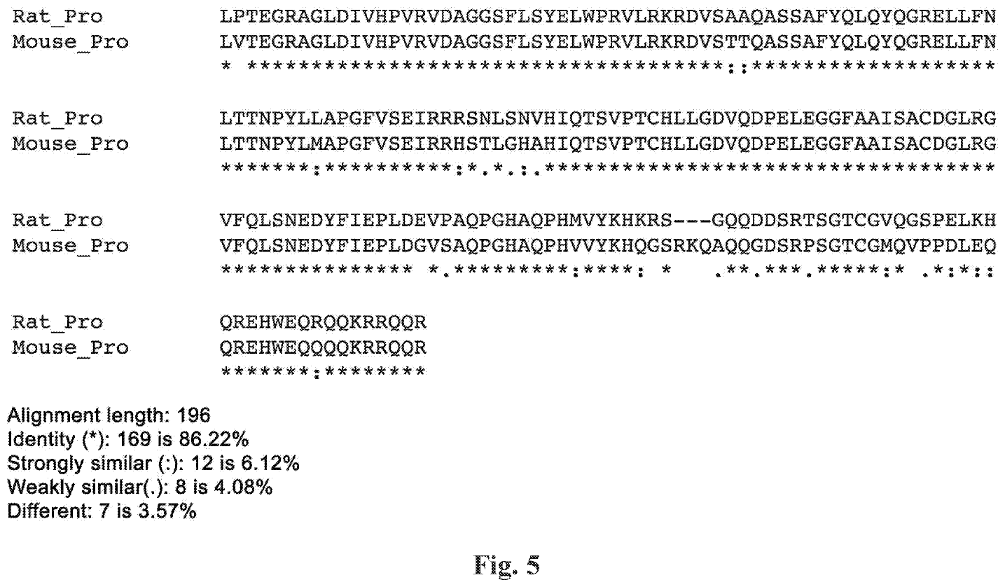

In some embodiments according to the first aspect, the first portion of the polypeptide comprises a mutation within the furin cleavage site. FIG. 4A and FIG. 4B show that furin cleavage site mutants of ADAMTS-7 improved the yield of processed or unprocessed ADAMTS-7. For example, in some of these embodiments, the motif RQQR is mutated to RQKR (Q216K). In some embodiments, the motif RQQR within the first portion of the polypeptide is altered, preferably into RQKR. Thus, in some embodiments, the first portion comprises a mutation, e.g. at position 216 with respect to SEQ ID NO: 1, such as the mutation Q216K. This mutation was found to improve cleavage by Furin between the prodomain and the catalytic domain of ADAMTS-7, thereby leading to improved yields of processed ADAMTS-7 compared to the wild type (WT). (e.g., Example 1, e.g., FIG. 4A and FIG. 4B).

In some embodiments, the first portion comprises triple mutant R58A/R61A/R217A (rPro-hCD-3RA). FIG. 4B shows that these mutations abolished the processing to generate unprocessed protein only.

According to some embodiments of the first aspect, the recombinant nucleic acid sequence encodes at least for a rat pro-domain of ADAMTS-7 (SEQ ID NO: 1, residues 1-217) and a CD domain of human ADAMTS-7, wherein optionally within the rat pro-domain of ADAMTS-7 the motif RQQR is mutated to RQKR (as in SEQ ID NO: 2, residues 1-217). In some of these embodiments, said rat pro-domain of ADAMTS-7 comprises or consists of a sequence according to SEQ ID NO: 1, residues 1-217 or SEQ ID NO: 2, residues 1-217 and/or said CD domain of human ADAMTS-7 comprises or consists of a sequence according to SEQ ID NO: 1, residues 218-518 or SEQ ID NO: 2, residues 218-518.

In some embodiments, the polypeptide or a fragment thereof encoded by the recombinant nucleic acid is suited to cleave a peptide comprising standard residues 1-15 of the amino acid sequence of SEQ ID NO: 4. For example, the polypeptide is suited to cleave a peptide comprising standard residues 1-15 of the amino acid sequence of SEQ ID NO: 4 and/or SEQ ID NO: 11 with a kcat/K_{M} of at least 20% of a corresponding kcat/K_{M} of human ADAMTS-7.

The produced polypeptide (e.g., having both the first portion and the second portion, or having only the second portion) can have a catalytic activity close to that of human ADAMTS-7 enzyme, e.g., in terms of kcat/K_{M}, which can be even higher than the kcat/K_{M} of the human enzyme, or can be within 50%, 45%, 40%, 35%, 30%, 25%, 20%, 15%, 10%, or 5% of the kcat/K_{M} of the human enzyme, for example when a peptide having the sequence of SEQ ID NO: 11 is used as the substrate.

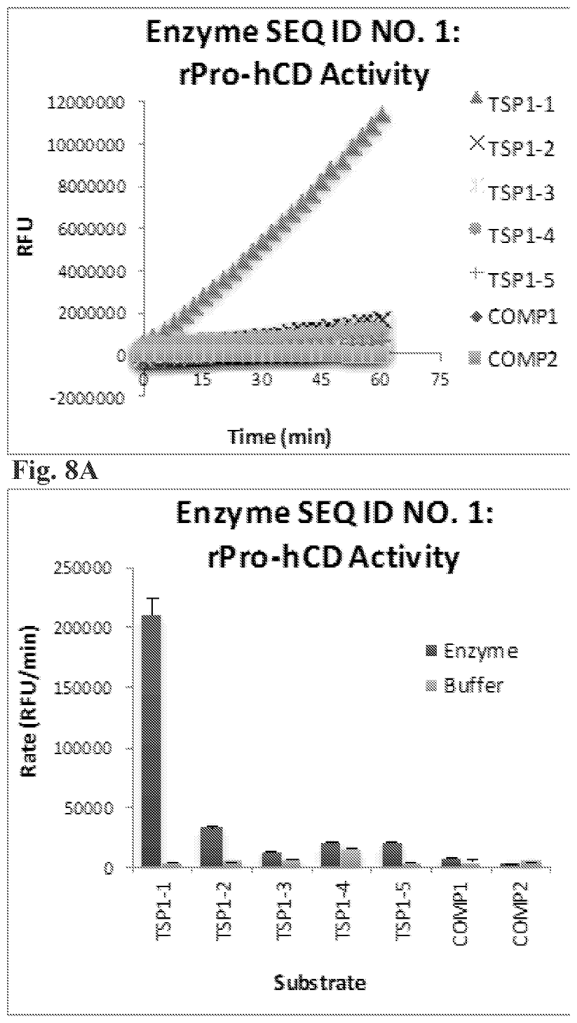

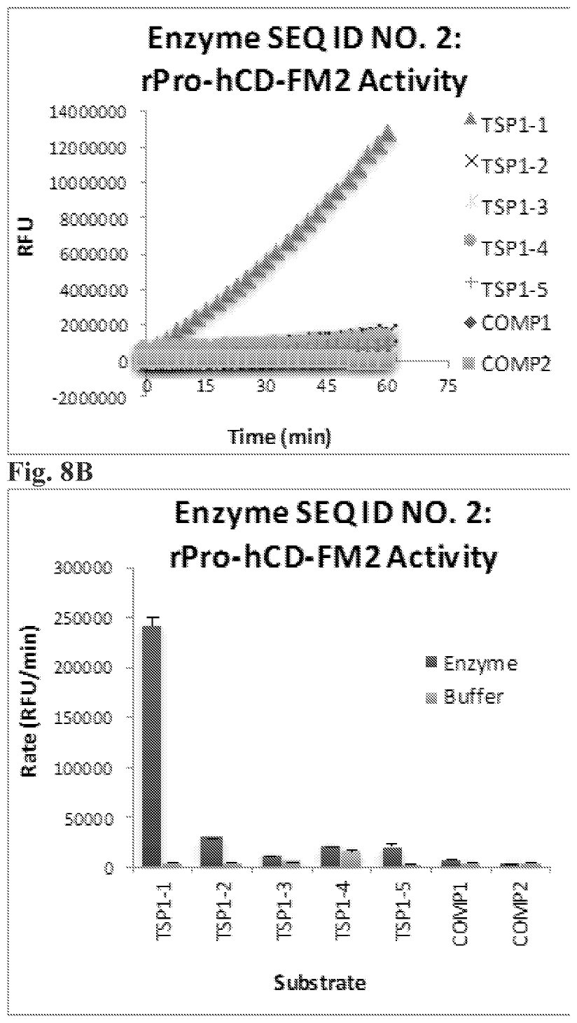

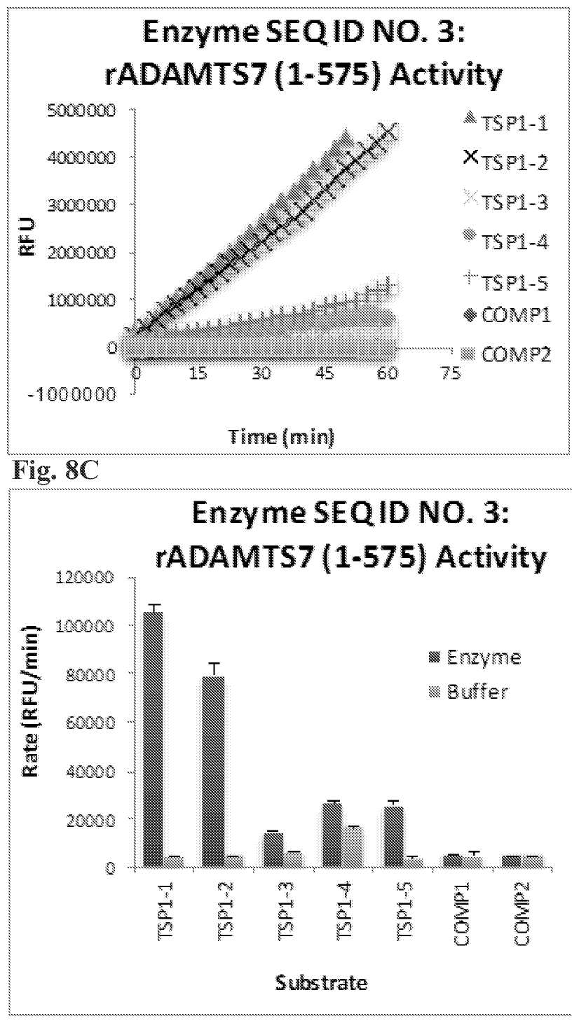

In some embodiments, for a given substrate, the produced polypeptide (e.g., having both the first portion and the second portion, or having only the second portion) has a catalytic activity in the same order of magnitude as rat ADAMTS-7, e.g., in terms of kcat, e.g., FIG. 8A-FIG. 8C and Table 3. In some embodiments, for a substrate of SEQ ID NO: 4, the produced polypeptide has a kcat (min⁻¹) in the order of magnitude of 10⁻². In some embodiments, for a substrate of SEQ ID NO: 5, the produced polypeptide has a kcat (min⁻¹) in the order of magnitude of 10⁻³.

Sequential cleavage or processing of ADAMTS-7 at furin cleavage sites by cellular enzyme furin leading to a complete removal of the Pro domain from the rest of the protein is likely a necessary step to a fully active or mature ADAMTS-7.

In some embodiments, the recombinant nucleic acid sequence furthermore encodes for additional residues, such as a purification tag, such as a FLAG tag, a His tag, a Strep tag or any combination or repetition thereof. In some embodiments of the first aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the aspect. Purification of a polypeptide obtained from a nucleic acid provided herein can occur as described in Example 3.

The recombinant nucleic acid according to the first aspect can be used for the expression of functional ADAMTTS7 as described in Example 3. For example, the construct can be inserted into a plasmid that is compatible with a certain expression system. In some embodiments, the construct can be cloned into a plasmid, preferably into a mammalian expression vector, such as pcDNA6mycHis. Expression can be performed in a compatible expression system, such as in a mammalian expression system, such as in HEK cells or Expi293 cells as known in the art. The Gibco Expi293 Expression System is a commercially available high-yield transient expression system based on suspension-adapted Human Embryonic Kidney (HEK) cells.

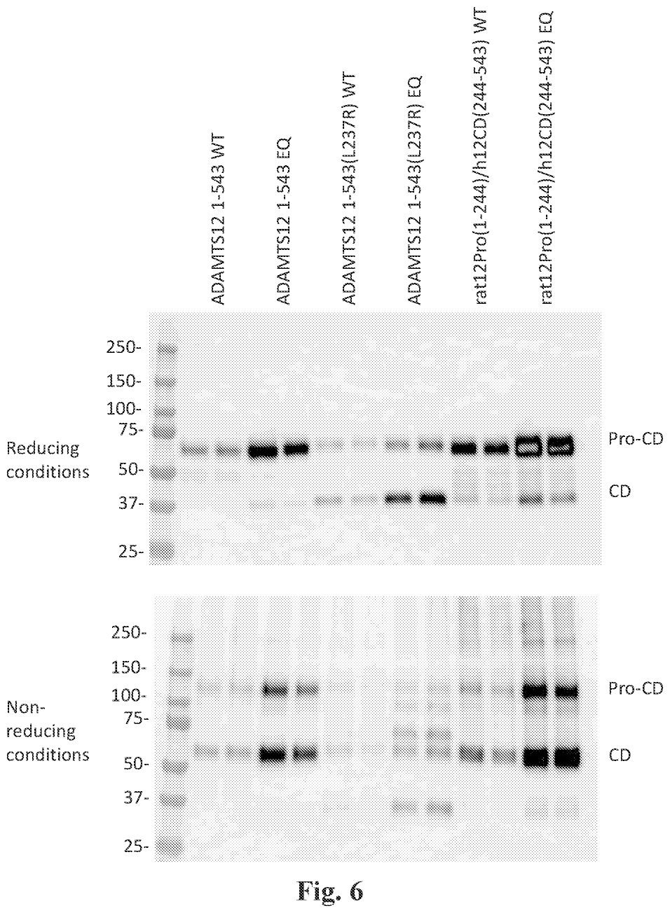

According to a second aspect, provided herein is a recombinant nucleic acid for the expression of functional ADAMTS-12. It was surprisingly found that human ADAMTS-12 expression is improved when a non-human prodomain is used (e.g., Example 9, e.g., FIG. 6). In particular, it was found that human ADAMTS-12 with a rodent prodomain demonstrated a better expression profile compared to human ADAMTS-12 with a human prodomain. The recombinant nucleic acids according to the second aspect are therefore suited for the improved production of sufficient amounts of ADAMTS-12 with reproducible activity and purity. The resulting recombinant functional ADAMTS-12 can be used in an assay for the characterization of modulators of ADAMTS-7, for example in which the selectivity for ADAMTS-7 is assessed.

According to some first embodiments according to the second aspect, there is provided a recombinant nucleic acid for expression of an ADAMTS-12 polypeptide that comprises a rodent prodomain of ADAMTS-12 as a first portion and a functional human ADAMTS-12 (e.g., a functional human ADAMTS-12 CD domain or catalytic domain) as a second portion.

In some embodiments according to the second aspect, the recombinant nucleic acid sequence encodes for a recombinant polypeptide that comprises a first portion and a second portion, wherein the first portion has a sequence identity of >80% with the sequence of residues 1-244 of SEQ ID NO: 15, and the second portion has a sequence identity of >80% with the sequence of residues 245-547 of SEQ ID NO: 15.

In some embodiments according to the second aspect, the recombinant nucleic acid sequence encodes for a recombinant polypeptide that comprises a first portion and a second portion, wherein the first portion has a sequence identity of >90% with the sequence of residues 1-244 of SEQ ID NO: 15, and the second portion has a sequence identity of >90% with the sequence of residues 245-547 of SEQ ID NO: 15.

In some embodiments according to the second aspect, the recombinant nucleic acid sequence encodes for a recombinant polypeptide that comprises a first portion and a second portion, wherein the first portion has a sequence identity of >95% with the sequence of residues 1-244 of SEQ ID NO: 15, and the second portion has a sequence identity of >95% with the sequence of residues 245-547 of SEQ ID NO: 15.

In some embodiments according to the second aspect, the recombinant nucleic acid sequence encodes for a recombinant polypeptide that comprises a first portion and a second portion, wherein the first portion has a sequence identity of >98% with the sequence of residues 1-244 of SEQ ID NO: 15, and the second portion has a sequence identity of >98% with the sequence of residues 245-547 of SEQ ID NO: 15.

In some embodiments, according to the second aspect, the recombinant nucleic acid sequence encodes for a recombinant polypeptide that comprises a first portion and a second portion, wherein the first portion comprises residues 1-244 of SEQ ID NO: 15, and/or the second portion comprises residues 245-547 of SEQ ID NO: 15. In some different or the same embodiments, the second portion is immediately C-terminal to the first portion.

In some embodiments according to the second aspect, the recombinant nucleic acid encodes a polypeptide that has a first portion and a second portion. The first portion of the polypeptide, in some embodiments, has an amino acid sequence that when aligned with an amino acid sequence of an ADAMTS-12 prodomain (or a fragment thereof) from a first species generates a Needleman-Wunsch score greater than 800 if BLOSUM62 matrix, a gap opening penalty of 11, and a gap extension penalty of 1 are used. In some embodiments, this generated Needleman-Wunsch score is greater than 850, 900, 950, 1000, 1050, 1100, 1150, 1200, 1250, or 1300. In some embodiments, the first portion and the ADAMTS-12 prodomain share a sequence identity that is at least 70%, 75%, 80%, 85%, 90%, 95%, 97% or 98%. The second portion of the polypeptide, in some embodiments, has an amino acid sequence that when aligned with an amino acid sequence of a human ADAMTS-12 CD domain (or a fragment thereof) generates a Needleman-Wunsch score greater than 1000 if BLOSUM62 matrix, a gap opening penalty of 11, and a gap extension penalty of 1 are used. In some embodiments, this generated Needleman-Wunsch score is greater than 1050, 1100, 1150, 1200, 1250, 1300, 1350, 1400, 1450, 1500, 1550, 1600, or 1650. In some embodiments, the second portion and the ADAMTS-12 CD domain share a sequence identity that is at least 70%, 75%, 80%, 85%, 90%, 95%, 97% or 98%. In some embodiments of these aspects, the second portion has an amino acid sequence that aligns with a functional segment of an ADAMTS-12 catalytic domain amino acid sequence from a second species with a Needleman-Wunsch score greater than 700 when BLOSUM62 matrix, a gap opening penalty of 11, and a gap extension penalty of 1 are used. In some embodiments, this generated Needleman-Wunsch score is greater than 750, 800, 850, 900, 950, 1000, 1050, 1100, or 1150. In some embodiments, the second portion and the ADAMTS-7 catalytic domain share a sequence identity that is at least 70%, 75%, 80%, 85%, 90%, 95%, 97% or 98%.

In some embodiments according to the second aspect, the recombinant nucleic acid for expression of an ADAMTS-12 polypeptide encodes for a recombinant polypeptide that comprises a first portion having an amino acid sequence that aligns with an amino acid sequence of a ADAMTS-12 prodomain or a fragment thereof from a non-human species with a Needleman-Wunsch score greater than 800 when BLOSUM62 matrix, a gap opening penalty of 11, and a gap extension penalty of 1 are used; and a second portion having an amino acid sequence that aligns with an amino acid sequence of a ADAMTS-12 CD domain or a fragment thereof from a second species with a Needleman-Wunsch score greater than 1000 when BLOSUM62 matrix, a gap opening penalty of 11, and a gap extension penalty of 1 are used. In some embodiments of this aspect, the second portion has an amino acid sequence that aligns with a functional segment of an ADAMTS-12 catalytic domain amino acid sequence from a second species with a Needleman-Wunsch score greater than 700 when BLOSUM62 matrix, a gap opening penalty of 11, and a gap extension penalty of 1 are used.

In some embodiments, the first species is a non-human species, such as a rodent species, such as rat. In some different or the same embodiments, the second species is human, e.g. the CD domain is derived from human ADAMTS-12. In some embodiments, the first species is a rodent species, and the second species is human.

In some embodiments, the second portion comprises a mutation at position E393, such as E393Q (EQ), with respect to SEQ ID NO: 24 (e.g., changing the sequence part AHEL to AHQL). This mutation results in increased protein yield similar to ADAMTS-7 catalytic mutations. In the Examples, unless specified otherwise, the references to residues and mutations are with respect to the wild-type forms of the proteins (e.g., SEQ ID NOs: 21 through 26). E393Q mutation, when specified with respect to SEQ ID NO: 15, corresponds to E397Q.

In some embodiments, the polypeptide or a fragment thereof encoded by the recombinant nucleic acid is suited to cleave a peptide comprising standard residues 1-15 of the amino acid sequence of SEQ ID NO: 4. For example, in some embodiments, the polypeptide is suited to cleave a peptide comprising standard residues 1-15 of the amino acid sequence of SEQ ID NO: 4 and/or SEQ ID NO: 11 with a kcat/K_{M} of at least 20% of a corresponding kcat/K_{M} of human ADAMTS-12.

The produced polypeptide (e.g., having both the first portion and the second portion, or having only the second portion) can have a catalytic activity close to that of human ADAMTS-12 enzyme (e.g., in terms of kcat/K_{M}, which can be even higher than the kcat/K_{M} of the human enzyme, or can be within 50%, 45%, 40%, 35%, 30%, 25%, 20%, 15%, 10%, or 5% of the kcat/K_{M} of the human enzyme, for example when a peptide having the sequence of SEQ ID NO: 4 or 11 is used as the substrate.

In some embodiments, the recombinant nucleic acid sequence furthermore encodes for additional residues such as a purification tag, such as a FLAG tag, a His tag, a Strep tag or any combination or repetition thereof. In some embodiments of the second aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the aspect. Purification of the functional ADAMTS-12 obtained from a recombinant nucleic acid provided herein can occur as described in Example 4. According to some embodiments of the second aspect, the recombinant nucleic acid sequence encodes at least for a rat pro-domain of ADAMTS-12, such as amino acids 1-244 of rat sequence UniProt D3ZTJ3 (from SEQ ID NO: 25) and/or encodes for a CD domain of human ADAMTS-12, such as amino acids 241-543 of human sequence UniProt P58397 (from SEQ ID NO: 24), e.g., Example 1. In certain embodiments, said recombinant nucleic acid encodes for a sequence according to SEQ ID NO: 15.

The recombinant nucleic acid according to the second aspect can be used for the expression of functional ADAMTTS-12 as described in Example 4. For example, the recombinant nucleic acid can be inserted into a plasmid that is compatible with a certain expression system. In some embodiments, the construct can be inserted into a plasmid, preferably into a mammalian expression vector, such as pcDNA3.4. Expression can be performed in a compatible expression system, such as in a mammalian expression system, such as in HEK cells or Expi293 cells as known in the art. The Gibco Expi293 Expression System is a commercially available high-yield transient expression system based on suspension-adapted Human Embryonic Kidney (HEK) cells.

According to a third aspect, provided herein is a recombinant polypeptide, wherein the recombinant polypeptide is the recombinant polypeptide encoded by a recombinant nucleic acid according to the first or second aspect, or a fragment thereof. In some embodiments, the fragment is the processed polypeptide which results after Furin cleavage. In some embodiments, the Furin cleavage occurs at the site known in the art or described herein.

In some embodiments, the recombinant polypeptide according to the third aspect or a fragment thereof is suited to cleave a peptide substrate comprising standard residues 1-15 of SEQ ID NO: 4. The recombinant polypeptide according to the third aspect or a fragment thereof can be used in an assay for the identification and characterization of modulators of ADAMTS-7 and/or ADAMTS-12, as shown within the Examples.

In some embodiments, the recombinant polypeptide according to the third aspect or a fragment thereof is a functional ADAMTS-7 protein. In some embodiments, the polypeptide comprises a rodent prodomain of ADAMTS-7 as a first portion and a functional human ADAMTS-7 (e.g., a functional human ADAMTS-7 CD domain or catalytic domain) as a second portion.

In some embodiments, the polypeptide comprises a first portion and a second portion, wherein the first portion has a sequence identity of >80% with the sequence of residues 1-217 of SEQ ID NO: 1 or with the sequence of residues 1-217 of SEQ ID NO: 2, and the second portion has a sequence identity of >80% with the sequence of residues 218-518 of SEQ ID NO: 1.

In some embodiments according to the third aspect, the recombinant polypeptide comprises a first portion having an amino acid sequence that aligns with an amino acid sequence of an ADAMTS-7 prodomain or a fragment thereof from a first species with a Needleman-Wunsch score greater than 700, when BLOSUM62 matrix, a gap opening penalty of 11, and a gap extension penalty of 1 are used; and a second portion having an amino acid sequence that aligns with an amino acid sequence of a ADAMTS-7 CD domain or a fragment thereof from a second species with a Needleman-Wunsch score greater than 1000 when BLOSUM62 matrix, a gap opening penalty of 11, and a gap extension penalty of 1 are used. In some embodiments, the first species is a rodent species such as rat and the second species is human. In some embodiments of these aspects, the second portion has an amino acid sequence that aligns with a functional segment of an ADAMTS-7 catalytic domain amino acid sequence from a second species with a Needleman-Wunsch score greater than 700 when BLOSUM62 matrix, a gap opening penalty of 11, and a gap extension penalty of 1 are used.

In some embodiments, the first portion of the polypeptide comprises a mutation within the furin cleavage site. In some embodiments, the motif RQQR within the first portion of the polypeptide is altered, preferably into RQKR. Thus, in some embodiments, the first portion comprises a mutation, e.g. at position 216, such as the mutation Q216K, with respect to SEQ ID NO: 1. This mutation was found to improve cleavage by Furin between the prodomain and the CD domain of ADAMTS-7, thereby leading to improved yields of processed ADAMTS-7 compared to the wild type (WT). (e.g., Example 1, e.g., FIG. 4A and FIG. 4B).

In some embodiments, the polypeptide comprises a rodent prodomain of ADAMTS-12 as a first portion and a functional human ADAMTS-12 (e.g., a functional human ADAMTS-12 CD domain or catalytic domain) as a second portion.

In some embodiments, the polypeptide comprises a first portion and a second portion, wherein the first portion has a sequence identity of >80% with the sequence of residues 1-244 of SEQ ID NO: 15, and the second portion has a sequence identity of >80% with the sequence of residues 245-547 of SEQ ID NO: 15.

In some embodiments according to the current aspect, the polypeptide comprises a first portion having an amino acid sequence that aligns with an amino acid sequence of a ADAMTS-12 prodomain or a fragment thereof from a first species with a Needleman-Wunsch score greater than 800 when BLOSUM62 matrix, a gap opening penalty of 11, and a gap extension penalty of 1 are used; and a second portion having an amino acid sequence that aligns with an amino acid sequence of a ADAMTS-12 CD domain or a fragment thereof from a second species with a Needleman-Wunsch score greater than 1000 when BLOSUM62 matrix, a gap opening penalty of 11, and a gap extension penalty of 1 are used. In some embodiments, the first species is a non-human species, such as a rodent species, such as rat. In some different or the same embodiments, the second species is human, e.g. the CD domain is derived from human ADAMTS-12. In some embodiments, the first species is a rodent species, and the second species is human. In some embodiments of this aspect, the second portion has an amino acid sequence that aligns with a functional segment of an ADAMTS-12 catalytic domain amino acid sequence from a second species with a Needleman-Wunsch score greater than 700 when BLOSUM62 matrix, a gap opening penalty of 11, and a gap extension penalty of 1 are used.

In some embodiments, the second portion comprises a mutation at position E397, such as E397Q (EQ), with respect to SEQ ID NO: 15. This mutation results in increased protein yield.

In some embodiments, the polypeptide (e.g., having both the first portion and the second portion, or having only the second portion) has a catalytic activity close to that of human ADAMTS-7 or ADAMTS-12 enzyme (e.g., in terms of kcat/K_{M}, which can be even higher than the kcat/K_{M} of the human enzyme, or can be 50%, 45%, 40%, 35%, 30%, 25%, 20%, 15%, 10%, or 5% of the kcat/K_{M} of the human enzyme, for example when a peptide having the sequence of SEQ ID NO: 4 or 11 is used as the substrate.

In some embodiments, the polypeptide or a fragment thereof is suited to cleave a peptide comprising standard residues 1-15 of the amino acid sequence of SEQ ID NO: 4. In some embodiments, the polypeptide is suited to cleave a peptide comprising standard residues 1-15 of the amino acid sequence of SEQ ID NO: 4 and/or SEQ ID NO: 11 with a kcat/K_{M} of at least 20% of a corresponding kcat/K_{M} of human ADAMTS-7 or human ADAMTS-12.

In some embodiments, the polypeptide comprises a purification tag, such as a FLAG tag, a His tag, a Strep tag or any combination or repetition thereof. In some embodiments of the first aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the aspect.

Surprisingly, the recombinant polypeptide according to the current aspect was found to fold into a functional ADAMTS, e.g. ADAMTS-7 or ADAMTS-12. Expression and purification of recombinant functional ADAMTS protein according to the third aspect can occur as described in Example 3.

According to a fourth aspect, provided herein is a recombinant peptide substrate for ADAMTS-7 and/or ADAMTS-12. The peptide substrate provided herein can be used as an artificial substrate for ADAMTS-7 and/or ADAMTS-12. The peptide substrate can furthermore be used in order to determine the proteolytic activity of ADAMTS-7 and/or ADAMTS-12, identify modulators of ADAMTS-7 and/or ADAMTS-12, and/or to determine the degree of modulation induced by an agonist or antagonist.

In some embodiments, the peptide substrate comprises a subsequence of a natural ADAMTS-7 and/or ADAMTS-12 substrate, such as a subsequence of TSP1 or COMP. In some embodiments, the peptide substrate according to the fourth aspect comprises at least a sequence according to any of SEQ ID NO: 4, 5, 8, 11, 12 or 13, or a fragment thereof comprising the amino acids EL. It was surprisingly found that theses sequences can be used as cleavage site for ADAMTS-7 and ADAMTS-12: For example, the sequence DELSSMVLELRGLRT, derived from TSP1, residues 275-289 has been surprisingly identified as a suitable substrate for ADAMTS-7 and ADAMTS-12. In some embodiments, cleavage occurs between Glu283 and Leu284 (EL).

In some embodiments, the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL.

In some embodiments, the peptide substrate according to the fourth aspect comprises at least the amino acids EL. In some embodiments, the peptide substrate according to the fourth aspect comprises at least the SEQ ID NO: 4, or a fragment thereof comprising the amino acids EL. In some embodiments, the peptide substrate according to the fourth aspect comprises at least the SEQ ID NO: 5, or a fragment thereof comprising the amino acids EL. In some embodiments, the peptide substrate according to the fourth aspect comprises at least the SEQ ID NO: 8, or a fragment thereof comprising the amino acids EL.

In some embodiments, the peptide substrate according to the fourth aspect comprises at least the SEQ ID NO: 11, 12 or 13 or a fragment thereof comprising the amino acids EL. It was surprisingly found that the solubility of the described peptide substrate according to the fourth aspect could be improved by including an additional hydrophilic moiety without affecting the activity profile (e.g., SEQ ID NO: 11, 12, 13).

In some embodiments, the peptide substrate according to the fourth aspect comprises a first moiety conjugated to a residue that is N-terminal to sequence fragment EL as comprised within SEQ ID NO: 4, 5, or 8 or the fragment thereof, and a second moiety conjugated to a residue that is C-terminal to said sequence fragment EL. In some embodiments, the first moiety comprises a fluorophore and the second moiety comprises a quencher, or the first moiety comprises a quencher and the second moiety comprises a fluorophore.

According to one theory that might be applicable, the fluorophore of the peptide substrate can be excited at a suitable wavelength, e.g. by exposing it to light. The suitability of a wavelength depends on the specific fluorophore and can be determined as known in the art. Without being bound by theory, the excited fluorophore transfers the energy to the closely located quencher, which has the ability to decrease the fluorescence intensity of the fluorophore, such that no fluorescence or only background fluorescence is detected at the emission wavelength of the fluorophore. If the peptide substrate provided herein is however exposed to or contacted with a functional ADAMTS-7 or ADAMTS-12, the enzyme cleaves the peptide, thereby separating fluorophore and quencher. In the absence of the quencher, the excited fluorophore emits light (e.g., more in the aggregate, or with a higher probability individually) in returning to the ground state and an increase in fluorescence can be detected.

Using a combination of functional ADAMTS-7 and the peptide substrate comprising a fluorophore and a quencher according to the fourth aspect as a substrate thus allows for the robust and reproducible identification and characterization of ADAMTS-7 modulators and/or ADAMTS-12 modulators.

The skilled person understands that according to the various aspects and embodiments provided herein, multiple sites can be used to attach the fluorophore and the quencher to the peptide as long as a) the distance between fluorophore and quencher allows for the transfer of energy between fluorophore and quencher and b) the ADAMTS-7 cleavage site is arranged in such a way that fluorophore and quencher are separated upon ADAMTS-7 cleavage of the peptide. The latter effect can be obtained by interposing the ADAMTS-7 cleavage site between the fluorophore and the quencher.

A variety of suitable pairs of fluorophores and quenchers have been described in the literature. The skilled person is well aware which pairs of fluorophore and quencher can be combined. To obtain a peptide according to the aspect at hand, the distance between fluorophore and quencher has to be adjusted to allow for the necessary transfer of energy as described herein. The specific distance depends on the specific selection of fluorophore and quencher and can be adjusted as known in the art. For example, in some embodiments according to the aspect at hand, EDANS as a fluorophore can be paired with DABCYL or DABSYL as a quencher. When EDANS and DABCYL are in a close proximity (10-100 Å), the energy emitted from EDANS will be quenched by Dabcyl, resulting in low or no fluorescence. However, if the compounds are separated EDANS will fluoresce. The optimal absorbance and emission wavelengths of EDANS are λabs=336 nm and λem=490 nm respectively, and for Dabcyl, the maximum absorbance wavelength is λabs=472 nm, which, to a large extent, overlap with the emission spectra of EDANS.

Another pair of fluorophore and quencher where the compounds can be used alone or in combination is 7-methoxy-coumarin-4-yl acetic acid (MCA) as the fluorophore with Lys(DNP) as a quencher. In some embodiments according to the aspect at hand, the fluorophore is MCA and the quencher is Lys(DNP). A further pair which can be used comprises 7-amino-4-carbamoylmethylcoumarin (ACC) as the fluorophore and 2,4-dinitrophenyl-lysine (Lys(DNP)) as the quencher. In some embodiments according to the aspect at hand, the fluorophore is ACC and the quencher is Lys(DNP). Another pair of fluorophore and quencher that can be used alone or in combination is HiLyteFluor, e.g. HiLyteFluor-488 as the fluorophore with QXL, e.g. QXL520 as a quencher. HiLyte Fluor fluorophores are commercially available for various wavelengths and can be prepared as known in the art (Jungbauer, L M et al. “Preparation of fluorescently-labeled amyloid-beta peptide assemblies: the effect of fluorophore conjugation on structure and function” Journal of molecular recognition: JMR vol. 22, 5 (2009): 403-13). QXL quenchers are commercially available for various wavelengths and can be prepared as known in the art. QXL 570 dyes are optimized quenchers for rhodamines (such as TAMRA, sulforhodamine B, ROX) and Cy3 fluorophores. Their absorption spectra overlap with the fluorescence spectra of TAMRA, sulforhodamine B, ROX and Cy3.

According to some embodiments according to the fourth aspect, said fluorophore is HiLyteFluor, such as HiLyteFluor-488 and the quencher is a matching QXL quencher, such as QXL520.

According to some embodiments according to the fourth aspect, said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is HiLyteFluor, such as HiLyteFluor-488 and the quencher is a matching QXL quencher such as QXL520.

According to some embodiments according to the fourth aspect, said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is MCA and said quencher is Lys(DNP).

According to some embodiments according to the fourth aspect, said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is ACC and said quencher is Lys(DNP).

In some embodiments, the fluorophore, such as HiLyte fluor 488 is attached to the N-terminus of the peptide and the quencher, such as QXL520, is attached to the C-terminus or vice versa.

In an embodiment according to the aspect at hand, an additional negative residue, such as carboxyl glutamic acid (in which the “carboxyl” simply denotes the position on the peptide) is attached C-terminal of the quencher, e.g. after the QXL520 quencher. The addition of the residue improved the solubility behavior of the peptide substrate and thereby lead to an improved reproducibility of the assay.

According to some embodiments according to the aspect at hand, said peptide substrate for ADAMTS-7 and/or ADAMTS-12 comprises or consists of a subsequence of a natural ADAMTS-7 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, and the peptide substrate is further characterized in comprising an additional negatively charged residue such as a carboxyl glutamic acid (in which the “carboxyl” simply denotes the position on the peptide), e.g. C-terminal of the quencher.

According to some embodiments according to the aspect at hand, said peptide substrate for ADAMTS-7 and/or ADAMTS-12 comprises or consists of a subsequence of a natural ADAMTS-7 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is HiLyteFluor, such as HiLyteFluor-488, said quencher is a matching QXL quencher such as QXL520 and the peptide substrate is further characterized in comprising an additional negatively charged residue such as a carboxyl glutamic acid, e.g. C-terminal of the quencher.

According to a fifth aspect, provided herein is a method for the identification or characterization of an ADAMTS-7 and/or ADAMTS-12 modulator comprising the steps of
a) contacting a recombinant polypeptide or a fragment thereof according to the third aspect, and
b) contacting said recombinant polypeptide or fragment thereof with a peptide substrate according to the fourth aspect, wherein the peptide substrate comprises a fluorophore and a quencher, and
c) detecting fluorescence as a measure for the activity of said recombinant polypeptide or a fragment thereof.

In some embodiments, the recombinant polypeptide is suited to cleave a peptide comprising standard residues 1-15 of the amino acid sequence of SEQ ID NO: 4.

In some embodiments, the recombinant polypeptide comprises a first portion and a second portion, the first portion having a sequence identity of at least 80% with the sequence of residues 1-217 of SEQ ID NO: 1 and/or having a sequence identity of at least 80% with the sequence of residues 1-217 of SEQ ID NO: 2, and the second portion having a sequence identity of >80% with the sequence of residues 218-518 of SEQ ID NO: 1 and the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL.

In some embodiments, the recombinant polypeptide comprises a first portion and a second portion, the first portion having a sequence identity of >80% with the sequence of residues 1-244 of SEQ ID NO: 15, and the second portion having a sequence identity of >80% with the sequence of residues 245-547 of SEQ ID NO: 15 and the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL.

In some embodiments, the recombinant polypeptide comprises a sequence according to SEQ ID NO: 1, SEQ ID NO: 2 or SEQ ID NO: 15 and/or the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, and optionally comprises a further carboxy group.

Contacting of different compounds can be performed by preparing a solution comprising the respective compounds, e.g. in an appropriate concentration, e.g. as described in the Examples. The test compound can be any molecule, such as any small molecule provided for throughout this application.

There is no specific order with regard to the steps of the method, except for the obvious restrictions resulting from the mode of action. In some embodiments, the method according to the aspect at hand is further characterized in that step c) is performed for a solution comprising said recombinant polypeptide, said test compound, and said peptide substrate.

In some embodiments, the method according to the aspect at hand can be used to determine the half maximal inhibitory concentration (IC50) or the half maximal effective concentration (EC50) of an ADAMTS-7 and/or ADAMTS-12 modulator. According to the FDA, IC50 represents the concentration of a drug that is required for 50% inhibition in vitro. The determined IC50 as described herein is a measure of the potency of the test compound in inhibiting ADAMTS-7 and/or ADAMTS-12 induced cleavage of a natural or artificial substrate. Half maximal effective concentration (EC50) refers to the concentration of a test compound which induces a response halfway between the baseline and maximum after a specified exposure time and can be used as a measure of agonistic potency of a compound. IC50 and EC50 can be determined as known in the art. In brief, the IC50 of a drug can be determined by constructing a dose-response curve and analyzing the resulting fluorescence for different concentrations of the test compound.

In some embodiments according to the fifth aspect, a test compound can be or is selected as a modulator, for example as an inhibitor, if after contacting the recombinant polypeptide with the at least one test compound according to step a) and after contacting said recombinant polypeptide with a peptide substrate according to step b) no significant increase of fluorescence is detected or if an increase of fluorescence is detected which is lower (e.g., significantly lower) than the increase of fluorescence which is detectable for a positive control in the absence of the test compound.

In some embodiments according to the fifth aspect, a test compound is selected as an ADAMTS-7 and/or ADAMTS-12 agonist if after contacting the recombinant polypeptide with the at least one test compound according to step a) and after contacting said recombinant polypeptide with a peptide substrate according to step b) an increase of fluorescence is detected (e.g., as high as for a positive control—for a full agonist; higher (e.g., significantly higher) than a positive control—for a superagonist; somewhat lower than but still constituting an increase—for a partial agonist) than the increase of fluorescence which is detectable for a negative control in the absence of the test compound. Various other available methods can be used to select a test compound as a particular type of a modulator (e.g., by constructing a concentration of the test compound vs. ADAMTS activity graph). An exemplary way how the method can be performed is described in Example 5.

In some embodiments according to the current aspect, there is provided a method for evaluating the selectivity of an ADAMTS-7 and/or ADAMTS-12 modulator comprising the method according to the fifth aspect further characterized in comprising the steps of
a) contacting a functional recombinant metalloproteinase different from ADAMTS-7 and ADAMTS-12 with the at least one test compound, and
b) contacting said functional recombinant metalloproteinase with a peptide substrate comprising a fluorophore and a quencher, wherein said peptide substrate comprises a cleavage site for said functional recombinant metalloproteinase different from ADAMTS-7 and/or ADAMTS-12; and
c) detecting the fluorescence as a measure for the activity of functional recombinant metalloproteinase different from ADAMTS-7 and ADAMTS-12.

In some embodiments, the functional recombinant metalloproteinase different from ADAMTS-7 and ADAMTS-12 is ADAMTS4, ADAMTS5, MMP12, MMP15, MMP2 or ADAM17 or any other enzyme listed in Table 4. In some embodiments, the peptide comprising a fluorophore and a quencher is the peptide as specified in Table 4 and/or comprises a cleavage site of said functional recombinant metalloproteinase different from ADAMTS-7 and/or ADAMTS-12 as disclosed in Table 4.

For characterization of the inhibition, based on the fluorescence IC50 values can be calculated from percentage of inhibition of enzyme activity as a function of test compound concentration.

According to a sixth aspect, provided herein is a modulator of ADAMTS-7 and/or ADAMTS-12 identified by a method according to the fifth aspect for use in the treatment of heart disease, vascular disease, and/or cardiovascular disease, including atherosclerosis, coronary artery disease (CAD), myocardial infarction (MI), peripheral vascular disease (PAD)/arterial occlusive disease and/or restenosis after angioplasty (including the use of drug-coated or non drug-coated balloons and/or stent-implantation) and/or for the treatment and/or prophylaxis of lung disease, inflammatory disease, fibrotic disease, metabolic disease, cardiometabolic disease and/or diseases/disease states affecting the kidneys and/or the central nervous and/or neurological system as well as gastrointestinal and/or urologic and/or ophthalmologic disease/disease states.

For these diseases or disease states, alterations or aberrant expression with regard to ADAMTS-7 and/or ADAMTS-12 have been described earlier. In some embodiments, the modulator of ADAMTS-7 and/or ADAMTS-12 is a modulator for use in the treatment of coronary artery disease (CAD), peripheral vascular disease (PAD) and myocardial infarction (MI). In some embodiments, the modulator according to the sixth aspect is a small molecule. In some embodiments, the modulator according to the seventh aspect is an antagonist of human ADAMTS-7. In some different or the same embodiments, the modulator according to the sixth aspect is an antagonist of human ADAMTS-12. In some different or the same embodiments, the modulator according to the sixth aspect is an antagonist of human ADAMTS4. In some embodiments, the modulator according to the sixth aspect is a small molecule, e.g. as provided in the Examples. While the identification of selective ADAMTS-7 and ADAMTS-12 modulators has been extremely challenging in the past, the provided assay now offers numerous means to obtain these modulators according to the current aspect.

According to a seventh aspect, provided herein is a method of producing a recombinant polypeptide according to any of the previous aspects, the method comprising cultivating a recombinant host cell comprising a recombinant nucleic acid according to any aspect described herein and recovering the recombinant polypeptide of a fragment thereof.

According to an eight aspect, provided herein is a kit of parts comprising a recombinant nucleic acid according to the first and/or second aspect and/or a polypeptide according to the third aspect and a peptide substrate according to the fourth aspect. In some embodiments, the kit of parts can be used to perform a method according to the fifth aspect in a convenient and reproducible way. In some embodiments, the provided kit of parts can be used to evaluate whether a test molecule is a modulator of ADAMTS-7. In some different or the same embodiments, the provided kit of parts can be used to evaluate whether a test molecule is a modulator of ADAMTS-12. In some embodiments, the provided kit of parts can be used to evaluate whether a test molecule is a modulator of ADAMTS-7 and a modulator of ADAMTS-12.

### EXAMPLES

### Example 1: Production of Human ADAMTS-7

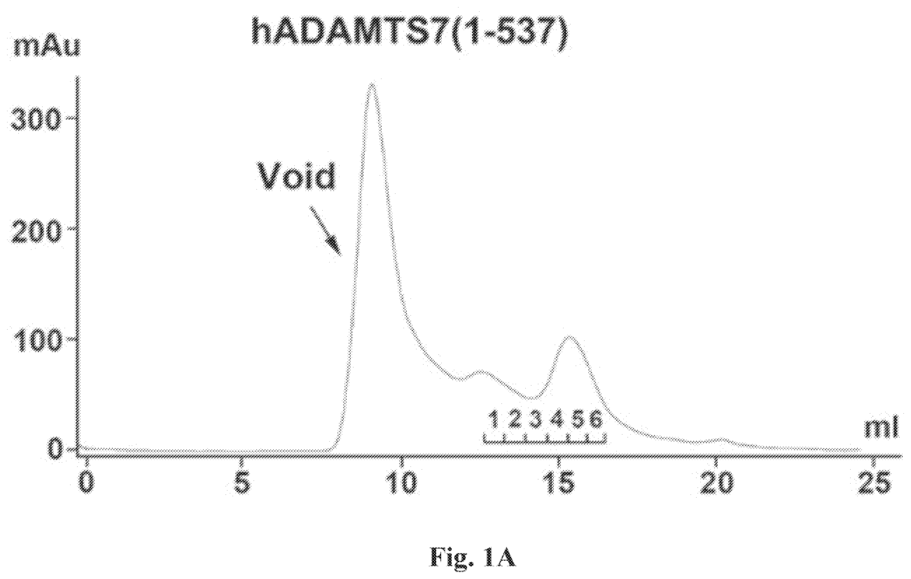

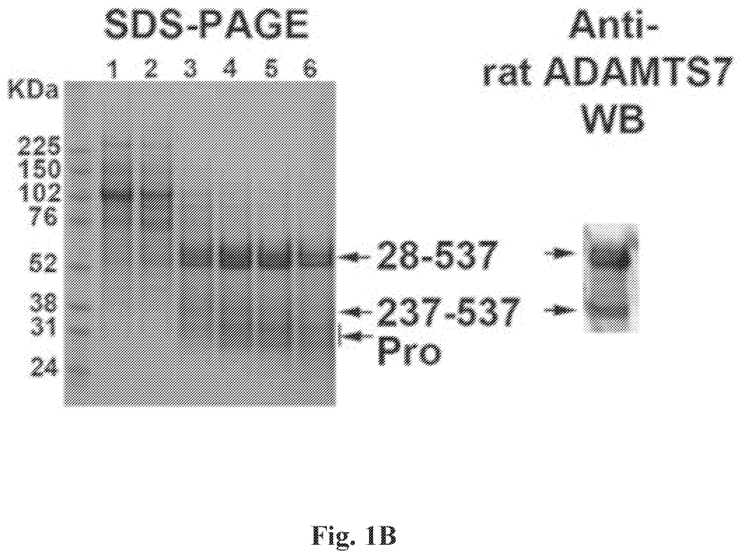

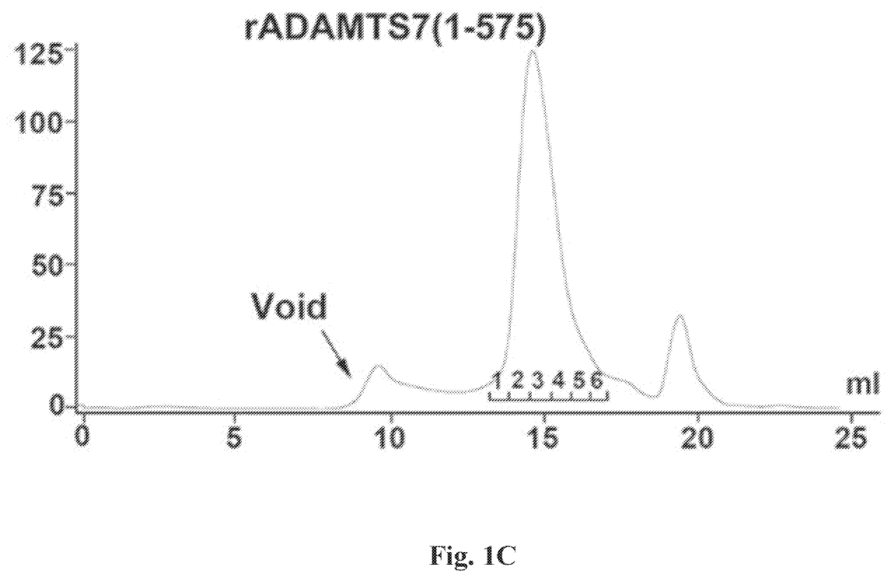

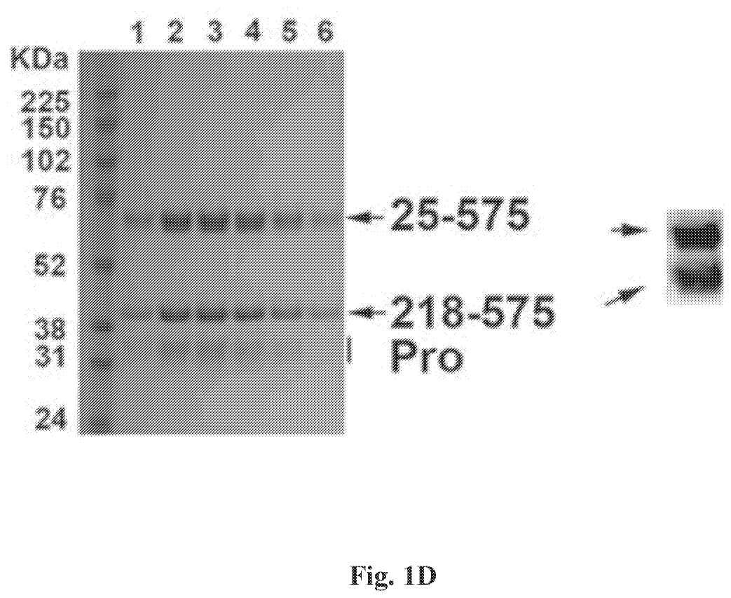

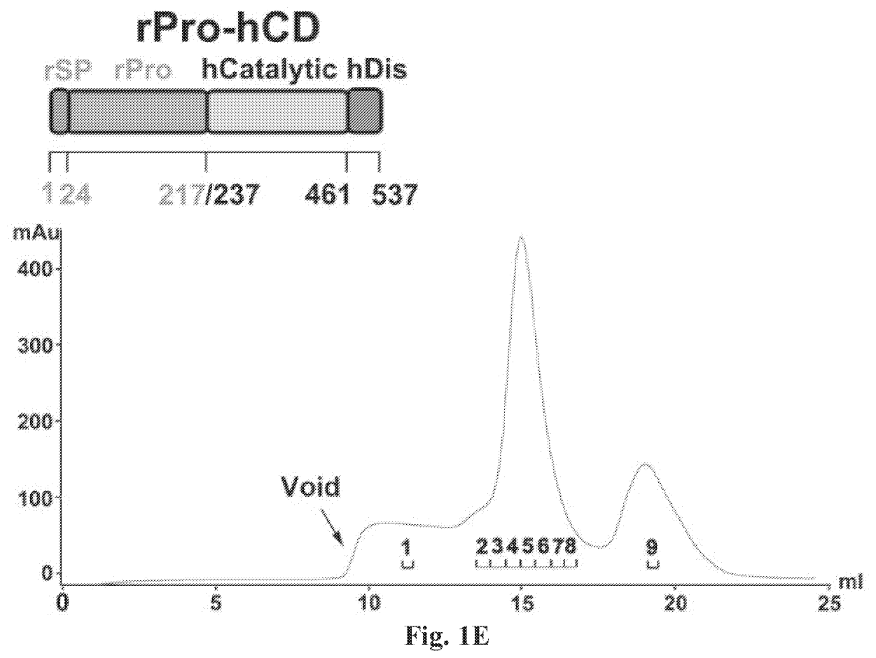

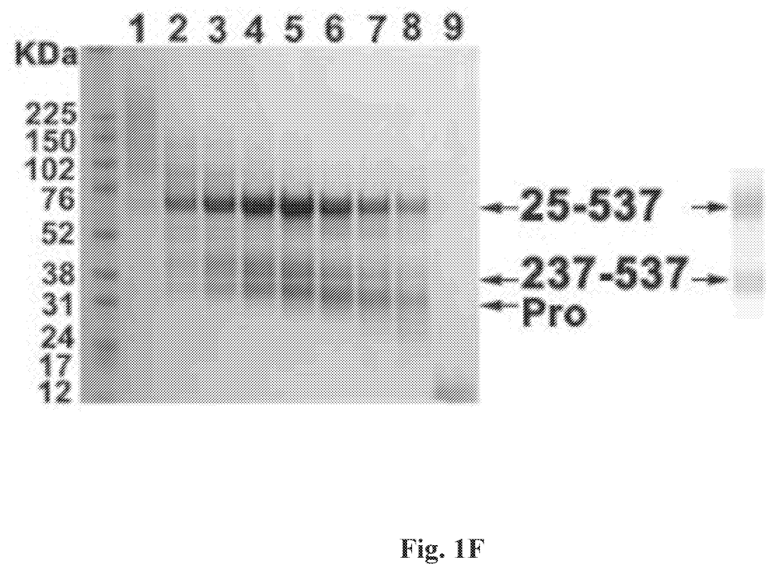

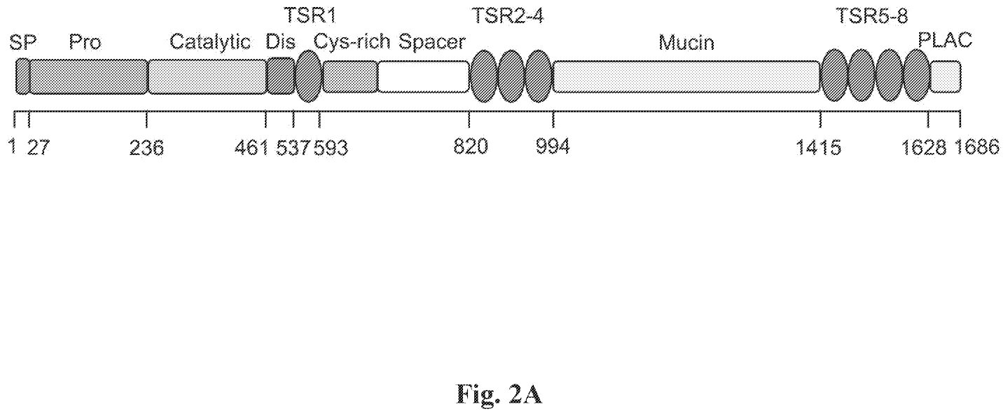

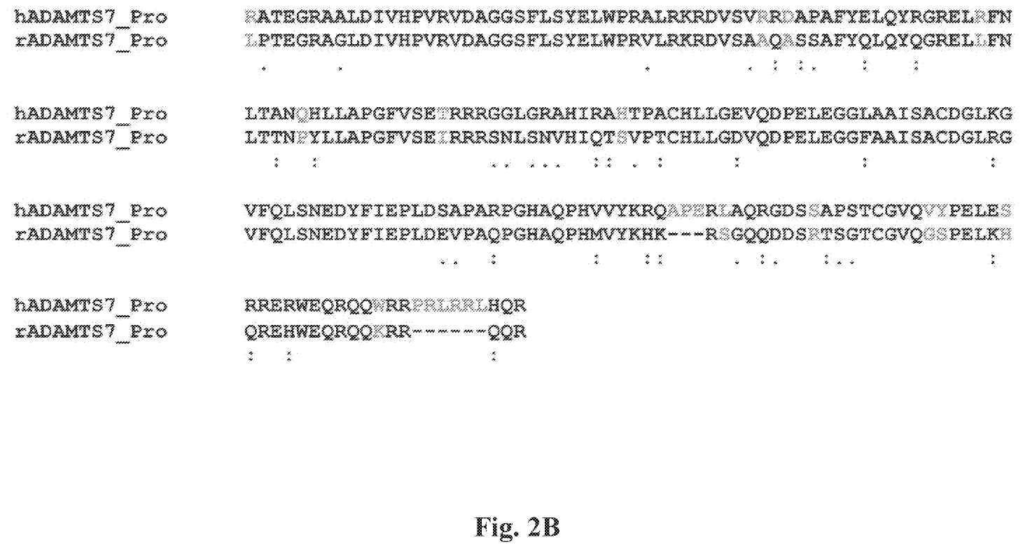

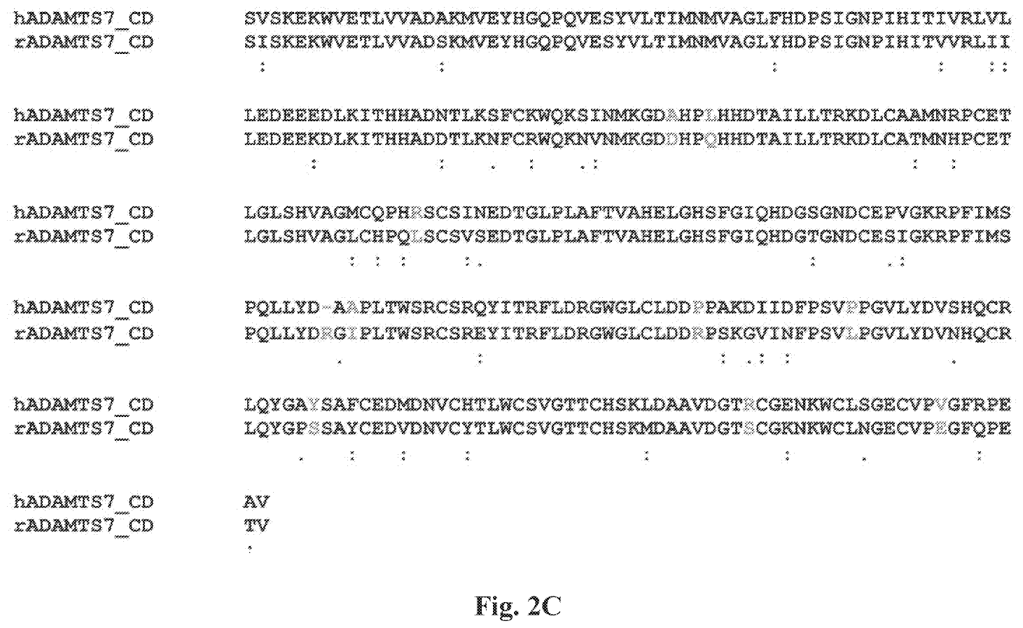

To study the activity of human ADAMTS-7 (hADAMTS-7), the inventors generated multiple constructs for the production of active ADAMTS-7 in E. coli cells. These constructs contain the catalytic domain alone or catalytic domain with the Prodomain (Pro), Disintegrin domain (Dis), or TSR1 (FIG. 2A). Various wild-type human, rat, and mouse ADAMTS-7 sequences are provided in SEQ ID NOs: 21-23. Different tags such as 6×His, GST, MBP, SUMO or Trigger factor (TF) were incorporated to the N-terminus of the constructs to improve the solubility of the protein and to facilitate protein purification, but none of these constructs produced soluble and active ADAMTS-7 to support further studies. Therefore, we tested catalytic domain containing constructs along with secretion signals in Expi293 mammalian cells. ADAMTS-7 proteins secreted into culture media were captured by affinity column and analyzed by analytical size exclusion column (SEC). A construct containing the secretion signal peptide (SP), Pro and CD domains of hADAMTS-7 (residues 1-537, from SEQ ID NO: 21, now referred to as “hPro-hCD” in light of the later discovered hybrids in which the prodomain can be from a non-human species) with an affinity tag at the C-terminus was eluted largely in the void volume from SEC and yielded ˜0.2 mg/L of soluble ADAMTS-7 proteins in the elution fractions 3-6 (FIG. 1A and FIG. 1B). Based on the size and N-terminal sequencing results of the bands on the SDS-page, the soluble ADAMTS-7 is confirmed to be a mixture of amino acid (aa) 28-537 (hPro-hCD, unprocessed ADAMTS-7), aa 237-537 (hCD domain only, furin processed ADAMTS-7), and fragments from Pro. The mixture was dominated by the unprocessed ADAMTS-7, which accounts for ˜90% of the population. A construct containing the secretion SP, an affinity/solubility tag and only hADAMTS-7 CD domain (residues 237-537) yielded mostly soluble aggregates (void). Soluble ADAMTS-7 proteins were only detectible by western blot (fractions B8-B12) (FIG. 3A). Another construct that ends at TSR1 (hSP-hPro-hCD-hTSR1, residues 1-593), instead failed to overexpress soluble ADAMTS-7 proteins. Neither SDS-PAGE nor western blot could detect significant ADAMTS-7 proteins (FIG. 3B). In comparison, rat ADAMTS-7 (rADAMTS-7) residues 1-575 (rSP-rPro-rCD-rTSR1) produced—0.6 mg/L soluble proteins (fractions 1-6) from Expi293 cells with negligible aggregates (void) eluted from SEC. Purified rat ADAMTS-7 contains—1:1 molar ratio of unprocessed (aa 25-575) and processed (aa 218-575) ADAMTS-7, and some fragments from Pro (FIG. 1C and FIG. 1D).

### Example 2: Hybrid Molecules to Improve the Production of Soluble hADAMTS-7

To improve the production of hADAMTS-7 protein, we also explored various other options. For example, we compared and analyzed the sequences of rat and human ADAMTS-7 in the propeptide (“Pro” in the figure, representing the prodomain without the signal peptide) and CD domains (FIG. 2B and FIG. 2C). CD domain sequence is well conserved. The amino acid sequence in the CD domain of rat and human is 84% identical (97% similar), compared to Propeptide which is 70% identical (89% similar). The rest of the sequences are different—neither identical nor similar—between the two species. Specifically, the CD domain has only 10 different residues between rat and human over 302 residues, while Pro has 22 different ones over 202 residues (FIG. 2B and FIG. 2C). To test whether rat Pro played a role in facilitating protein folding and yielding more soluble ADAMTS-7 proteins, we designed hybrid molecules of rat SP-Pro (1-217) followed by human CD (237-537), named rPro-hCD (FIG. 1E and FIG. 1F). The hybrid molecule was purified by Ni affinity column and analyzed by SEC as done to hADAMTS-7 (1-537). Little was eluted as aggregates in the void volume preceding the soluble peak (fractions 2-8). The soluble proteins were analyzed as a mixture of the unprocessed (aa 25-537) and processed (aa 237-537) ADAMTS-7, and Pro (FIG. 1E and FIG. 1F). The yield of ADAMTS-7 protein after affinity and SEC two-step purification was 2.2 mg/L for rPro-hCD. This is in contrast with ˜0.2 mg/L yield of the hPro-hCD, namely hADAMTS-7 (1-537). Our result suggests that the rat Pro is more effective in driving effective folding of the CD domain, therefore improving the yield of the soluble ADAMTS-7 proteins ˜10 fold. Therefore, this Example demonstrates a solution for the problem of protein solubility as well as for the problem of expression yield, both of which were lower for the fully human construct (hPro-hCD) as compared to the hybrid construct (rPro-hCD).

### Example 3: Engineering Furin Cleavage Site to Manipulate the Production Ratio of the Processed and Unprocessed ADAMTS-7

We hypothesized multiple furin cleavage sites in rat Pro (FIG. 4A), one after residue R58 or R60 (RVLR⁵⁸⬇KR⁶⁰⬇D) of Pro, and another between Pro and CD domains after R217 (RQQR²¹⁷⬇S). These cleavage sites were found conserved in rat, mouse, and human Pro. Sequential cleavage or processing at these furin sites by cellular furin enzyme leading to a complete removal of the Pro domain from the rest of the protein is likely a necessary step to a fully active or mature ADAMTS-7. Recombinant production of hPro-hCD and rPro-hCD revealed an inefficient furin processing in the mammalian cell culture (FIG. 1A, FIG. 1B, FIG. 1E, and FIG. 1F). Both constructs yielded a mixture of processed and unprocessed ADAMTS-7. We hypothesized that this could be attributed to a less sufficient amount of endogenous furin produced by mammalian cells or less optimal furin recognition sequence in current ADAMTS-7 constructs. We tested the idea of co-transfection DNAs of rPro-hCD and furin protease into Expi293 cells to coexpress the two proteins. That yielded overexpression of furin in the media but significantly reduced the production of ADAMTS-7 proteins without increasing the ratio of processed versus unprocessed ADAMTS-7. Next, we focused on the optimization of furin recognition site. We introduced mutation Q216K into rPro-hCD to convert ²¹⁴RQQR²¹⁷ to ²¹⁴RQKR²¹⁷, and named it rPro-hCD-FM2 (SEQ ID NO: 2) (FIG. 4A). Q216K mutation significantly increased furin cleavage efficiency of ADAMTS-7 in the same mammalian cell culture compared to the wild type (WT). The ratio of hCD or processed ADAMTS-7 in the raw expression media versus rPro-hCD or unprocessed ADAMTS-7 increased at least 6 fold (FIG. 4B). As a control, a triple mutation R58A/R60A/R217A was introduced into rPro-hCD to silence predicted furin cleavage sites (FIG. 4A). rPro-hCD-3RA completely abolished furin processing and yielded only the unprocessed ADAMTS-7 (FIG. 4B). We conclude that manipulating the sequence of predicted furin recognition site has proved capable of changing the amount of processed and unprocessed of ADAMTS-7 molecules produced in mammalian cell culture. Thus, both the polypeptide of SEQ ID NO: 1 and that of SEQ ID NO: 2 are working solutions for the problems of protein solubility and expression yield, although the polypeptide of SEQ ID NO: 2 achieves a particularly improved ratio for the furin-processed polypeptide.

### Example 4: Expression Constructs for Production of Recombinant Hybrid ADAMTS-7 Enzymes

The human ADAMTS-7 catalytic domain with the endogenous human prodomain did not yield well folded secreted protein with enzymatic activity. Exchange of the human prodomain with the rat prodomain dramatically increased production of the human catalytic domain from hybrid rat-human constructs. Rat/human ADAMTS-7 chimera sequence (rPro-hCD (Rat 1-217/Human 237-537)-TEV-2Strep-6His, SEQ ID NO: 1) encoding rat pro-domain of ADAMTS-7 (amino acids 1-217 of rat sequence UniProt Q1EHB3, which also includes the signal peptide) and catalytic domain of human ADAMTS-7 (amino acids 237-537 of human sequence UniProt Q9UKP4, which also includes a disintegrin domain) followed by TEV cleavage sequence, 2×Strep tag and a His Tag was cloned into the mammalian pcDNA6mycHis (ThermoFischer Scientific) expression vector (or into pcDNA3.4 vector in some embodiments).

Rat/human ADAMTS-7 chimera sequence (rPro-hCD-FM2 (Rat 1-217/Human 237-537 FM2 (Q216K))-TEV-2Strep-6His, SEQ ID NO: 2) encoding rat pro-domain of ADAMTS-7 (amino acids 1-217 of rat sequence UniProt Q1EHB3, which also includes the signal peptide) and catalytic domain of human ADAMTS-7 (amino acids 237-537 of human sequence UniProt Q9UKP4, which also includes a disintegrin domain) followed by TEV cleavage sequence, 2×Strep tag and a His Tag was cloned into the mammalian pcDNA6mycHis (ThermoFischer Scientific) expression vector (or into pcDNA3.4 vector in some embodiments). SEQ ID NO: 2 contains an additional mutation Glutamine 216 to Lysine within the rat pro-domain sequence (RQQR217⬇S to RQKR217⬇S), which improved cleavage by Furin for zymogen processing.

These expression constructs allow production of recombinant ADAMTS-7 enzyme—either containing at least parts of both domains (e.g., prodomain plus catalytic domain as encoded, in which the prodomain can optionally be preceded by a signal peptide and/or the catalytic domain can optionally be followed by a disintegrin domain) or containing primarily the catalytic domain (e.g., catalytic domain as encoded, for example as generated after furin cleavage between the prodomain and the catalytic domain, which catalytic domain can optionally be followed by a disintegrin domain).

### Example 5: Recombinant Production of Active ADAMTS-7 Enzyme

Expi293 cells (A14635, ThermoFischer Scientific) were grown and transfected in accordance to the manufacturer instruction. In brief, at the final cell density of 2.5×10⁶ cells/mL with >95% viability Expi 293 cells were transfected with 1 mg/liter of vector plasmid DNA as generated according to Example 4 using Expifectamine transfection reagent (Thermo Fischer Scientific).

Approximately 96 hours post transfection, Expi293 cell culture was centrifuged at 4000 rpm (˜3700 rcf) for 10 mins, and supernatant was collected. Supernatant was neutralized with 50 mM Tris pH8.0, 5 mM CaCl₂, 10 uM ZnCl₂ and 5 mM imidazole pH 8.0 and filtered through 0.22 μm filter.

The filtered supernatant was loaded on Ni-NTA column (GE healthcare #17-3712-06), equilibrated with buffer A (50 mM Tris pH 8.0, 300 mM NaCl, 10% glycerol, 5 mM CaCl₂, 10 μM ZnCl₂ and 5 mM Imidazole (pH 8.0)) on Acta FPLC system. The column was washed with 20 volumes of buffer A and the bound proteins were eluted by linear gradient of 20 column volumes up to 100% buffer B (50 mM Tris pH 8.0, 300 mM NaCl, 10% glycerol, 5 mM CaCl₂, 10 μM ZnCl₂ and 500 mM Imidazole (pH 8.0)). The collected fractions were analyzed on the SDS gel and the fractions containing ADAMTS-7 protein were combined and concentrated 10 times.

The concentrated material from the Ni-NTA purification was loaded onto superdex S200 (SEC) column equilibrated in column buffer: 50 mM Tris pH 8.0, 300 mM NaCl, 10% glycerol, 5 mM CaCl₂ and 10 μM ZnCl₂. The collected fractions were analyzed on the SDS gel and the fractions containing ADAMTS-7 protein were combined and concentrated 10 times.

The concentrated material from the Ni-NTA purification was loaded onto superdex S200 (SEC) column equilibrated in column buffer: 50 mM Tris pH 8.0, 300 mM NaCl, 10% glycerol, 5 mM CaCl₂ and 10 μM ZnCl₂. The collected fractions were analyzed on the SDS gel and the fractions containing ADAMTS-7 protein were combined.

Combined S200 fractions were loaded to strep-tactin column (Qiagen #1057981) equilibrated in Buffer A (50 mM Tris 8.0, 300 mM NaCl, 10% glycerol, 5 mM CaCl₂, 10 μM ZnCl₂). The column was washed with 20 column volumes of the buffer A and the bound proteins were eluted by linear gradient of 20 column volumes up to 100% buffer B (50 mM Tris 8.0, 300 mM NaCl, 10% glycerol, 5 mM CaCl₂, 10 uM ZnCl₂ and 2.5 mM D-desthiobiotin). The collected fractions were analyzed on the SDS gel and the fractions containing ADAMTS-7 protein were combined. Combined fractions were dialysed overnight at +4° C. against the storage buffer (20 mM Tris pH 8.0, 300 mM NaCl, 10% glycerol, 5 mM CaCl₂ and 10 μM ZnCl₂).

Dialyzed protein was concentrated to 1 mg/ml, aliquoted, flash frozen in dry ice/ethanol and stored at −80° C. The final yield of purified ADAMTS-7 was 0.5-1 mg per liter of Expi293 cell culture.

In some embodiments, the produced polypeptide is further processed (e.g., by TEV protease) to remove some of the parts C-terminal to the catalytic domain.

### Example 6: Identification of ADAMTS-7 Substrates and Cleavage Assay Development

Based on the interaction mapping data and the apparent fragment size in reducing and non-reducing conditions, the results were consistent with an ADAMTS-7 cleavage site near the second EGF repeat of COMP, however the substrate cleavage site has not been defined.

In an attempt to identify ADAMTS-7 substrate cleavage sites, we scanned the potential regions of COMP and TSP1 to identify ADAMTS consensus sites and generated a series of internally-quenched fluorescently-labeled peptides for use with our purified ADAMTS-7 enzyme (FIG. 7A and FIG. 7B). Since ADAMTS-7 was reported to produce the 100 kDA COMP fragment resolvable on a gel under reducing and non-reducing conditions, we hypothesized that cleavage site would not be internal to a disulfide bond. Given the 1-3, 2-4, 5-6 ensemble of disulfide bonds within the EGF repeats, only the E152⬇A153 site between the 4^{th} and 5^{th} cysteines committed to separate disulfide bonds was considered, which we attempted to emulate using acetamidomethyl (Acm) modified cysteines in the COMP candidate peptide (See FIG. 7A and FIG. 7B) A second peptide from COMP (amino acids 73-84 GMQQ⬇SVRTGLPS) was chosen based on observed ADAMTS cleavage of full-length COMP identified by N-terminal sequencing (data not shown). TSP1 contained a potential E⬇LRG upstream from the ADAMTS1 cleavage site at E311⬇L312. We chose to analyze this portion of TSP1 using a series of overlapping peptides. The COMP and TSP1 candidate regions contained a modified rhodamine AF488 dye coupled to the N-terminus and a local acceptor QXL520 quencher at the C-terminus that prevents fluorescence in the uncleaved configuration (FIG. 7A and FIG. 7B). Following endopeptidic cleavage, the quencher is released to allow fluorescence signal detection from the substrate's amino terminus.

Purified recombinant ADAMTS-7 (as of SEQ ID NO: 1 or SEQ ID NO: 2) and purified recombinant rADAMTS-7 (rat ADAMTS-7 residues 1-575 with a carboxyl terminal Flag, SEQ ID NO: 3) were used to identify substrates from the TSP1 and COMP candidate peptides. ADAMTS-7 was diluted in reaction buffer (20 mM HEPES pH 8.0, 150 mM NaCl, 5 mM CaCl₂, 0.004% Brij, 10 μM ZnCl₂) for a concentration of approximately 100 nM and pre-incubated for 2 hr at room temperature prior to reaction start with 10 μM candidate peptide substrates. There were some differences between optimized buffer data and reported 2 mM Zn²⁺ buffer data (Liu 2006 FASEB J); for example, the published amount of Zinc in assay buffer caused the purified ADAMTS-7 proteins to precipitate. Candidate FRET substrates based on SEQ ID NO: 4 to 10 were generated through custom synthesis by Anaspec—AnaSpec, EGT (34801 Campus Drive, Fremont, Calif. 94555, USA); as explained by the manufacturer; AnaSpec, EGT Group's pH insensitive HiLyte™ Fluor dyes are a series of fluorescent labeling dyes with fluorescence emissions that span the full visible and near infrared spectrum. HiLyte™ Fluor dyes and AnaSpec's proprietary quenchers QXL™ have been used as fluorophore and quencher pairs for fluorescence resonance energy transfer (FRET) in our Examples in the reaction buffer. Activity data plotted as Relative Fluorescence Units over time and as a calculated rate (RFU/min) are shown in FIG. 8A-FIG. 8C. Significant activity was not observed with substrates in buffer alone (FIG. 8A-FIG. 8C) or with incubation with purified ADAMTS-7 E389Q catalytic inactive proteins (data not shown; residues numbered with respect to SEQ ID NO: 21).

ADAMTS-7 human catalytic domain constructs rPro-hCD hybrid (SEQ ID NO: 1) and optimized furin site construct FM2 (SEQ ID NO: 2) demonstrated the greatest specificity for the TSP1 S1 (containing amino acids 275-289: DELSSMVLELRGLRT, amino acids 1-15 of SEQ ID NO: 4). ADAMTS-7 human catalytic domain constructs also cleaved the overlapping TSP1 S2 substrate (containing amino acids 278-292: SSMVLELRGLRTIVT, amino acids 1-15 of SEQ ID NO: 5). Substrates 51 and S2 are overlapping at candidate site to E289⬇L290 (FIG. 7A and FIG. 7B). Cleavage at this site was confirmed by mass spec using an unlabeled peptide (data not shown). No significant activity was detected at the defined ADAMTS1 cleavage site E311⬇L312 from TSP1 S3 (containing amino acids 300-314: KVTEENKELANERR, amino acids 1-15 of SEQ ID NO: 6) or TSP1 S4 (containing amino acids 303-317 of human TSP1: EENKELANERRPPL, amino acids 1-15 of SEQ ID NO: 7). This suggests that the ADAMTS-7 substrate is distinct from ADAMTS1. To confirm the TSP1 E2891⬇L290 substrate site, a minimum overlap between the 51 and S2 peptides was tested as TSP1 S5 substrate (containing amino acids 278-289: SSMVLELRGLRT, amino acids 1-12 of SEQ ID NO: 8). Activity was observed for the S5 substrate with ADAMTS-7 human catalytic domain constructs compared to buffer alone, however the removal of the amino DEL sequence greatly reduced the signal for activity compared to the 51 substrate containing DELSSMVLELRGLRT, amino acids 1-15 of SEQ ID NO: 4. No activity was observed with COMP candidate peptides COMP1 amino acids 73-84: GMQQSVRTGLPS, SEQ ID NO: 9 or COMP2 amino acids 146-159: SPGFRCEACPPGYS, SEQ ID NO: 10.

Activity data from the rat ADAMTS-7 construct identified TSP1 S1 (SEQ ID NO: 4) as the substrate, along with S2 and S5 peptides containing the E289⬇L290 cleavage site (FIG. 8A-FIG. 8C). 51 relative fluorescence signal was not as strong for rat ADAMTS-7 compared to the ADAMTS-7 human catalytic domain constructs, resulting in a lower ratio of S1 to S2 activity.

Table 3 provides the specific activities for the proteolysis of substrate SEQ ID NOs: 4-10 and shows that TSP1-1 substrate is most efficiently turned over by the active ADAMTS-7 constructs. The TSP substrate sequences indicate the cleavage site (underlined) between glutamate and leucine as determined by mass spectrometry. COMP substrate cleavage products were below the detection limit of the mass spectrometer.

TABLE 3

| | | | | k_{cat} (min⁻¹⁾ | | |
|---|---|---|---|---|---|---|
| | | | | Enzyme | Enzyme | Enzyme |
| | | | | SEQ ID | SEQ ID | SEQ ID |
| SEQ | | | | NO: 1: | NO: 2 : | NO: 3: |
| NO: | Sub- | | | rPro- | rPro- | rADAMTS7 |
| ID | strate | aa# | Sequence | hCD | hCD-FM2 | (1-575) |
| 4 | TSP1-1 | 275-289 | DELSSMVL | 2.1E−02 | 2.4E−02 | 1.0E−02 |
| | | | ELRGLRT | | | |
| 5 | TSP1-2 | 278-292 | SSMVLELR | 3.2E−03 | 2.9E−03 | 7.7E−03 |
| | | | GLRTIVT | | | |
| 6 | TSP1-3 | 300-314 | KVTEENKEL | 1.2E−03 | 1.2E−03 | 1.4E−03 |
| | | | ANELRR | | | |
| 7 | TSP1-4 | 303-317 | EENKELANE | 2.0E−03 | 2.1E−03 | 2.6E−03 |
| | | | LRRPPL | | | |
| 8 | TSP1-5 | 278-289 | SSMVLEL | 2.0E−03 | 2.1E−03 | 2.5E−03 |
| | | | RGLRT | | | |
| 9 | COMP1 | 73-84 | GMQQSVRT | 7.3E−04 | 7.6E−04 | 4.9E−04 |
| | | | GLPS | | | |
| 10 | COMP2 | 146-159 | SPGFRC | 3.8E−04 | 3.9E−04 | 5.0E−04 |
| | | | (Acm) | | | |
| | | | EAC(Acm) | | | |
| | | | PPGYS | | | |

TSP1 S2 substrate (containing SSMVLELRGLRTIVT, amino acids 1-15 of SEQ ID NO: 5) presented limited solubility compared to the TSP1 S1 substrate (containing DELSSMVLELRGLRT, amino acids 1-15 of SEQ ID NO: 4), potentially due to the additional hydrophobic residues at the carboxyl terminal side (data not shown). To further improve solubility of the 51 peptide, which contained a number of internal hydrophobic residues, modified versions of the 51 peptide ending in -K(Q)<L520)-NH2 were generated to include an additional hydrophilic moiety: -K(Q)<L520)-E-NH2 (SEQ ID NO: 11), -K(QXL520)-K-NH2 (SEQ ID NO: 12) and -K(QXL520)-OH (SEQ ID NO: 13). Activity profiles for these substrates were not significantly affected, however the substrate solubility profile was improved with the additional carboxyl glutamic acid (i.e., a glutamic acid that has been conjugated at a carboxyl position on the peptide) added after the QXL520 quencher for SEQ ID NO: 11 (data not shown).

### Example 7: Assay for ADAMTS-7 Enzymatic Activity and Testing of Inhibitory Compounds

Purified recombinant ADAMTS-7 (as of SEQ ID NO: 1 or SEQ ID NO: 2) was diluted in reaction buffer (20 mM HEPES pH 8.0, 150 mM NaCl, 5 mM CaCl₂, 0.004% Brij, 10 μM ZnCl₂) for a concentration of approximately 20 nM. 25 μl of the solution were transferred into each well of a 384-well white microtiter plate (Greiner Bio-One 781075) and 1 μl test compound solution (modulator/inhibitor dissolved in DMSO, at the corresponding concentration) or pure DMSO as a control were added per well. The enzymatic reaction was initiated by addition of 25 μl of a 1 μM solution of the FRET substrate, HiLyteFluor-488 DELSSMVLELRGLRT-K(QXL520)-E-NH2; (SEQ ID NO: 11, custom synthesis by Anaspec) in the reaction buffer. Amino acids DELSSMVLELRGLRT are derived from Thrombospondin-1 sequence (275-289). An additional carboxyl glutamic acid was added after the QXL520 quencher to increase substrate solubility. The microtiter plate was incubated for 120 min at the temperature of 32° C. The increase of fluorescence intensity was measured in appropriate fluorescence plate reader (e.g. TECAN Ultra) using excitation wavelength of 485 nm and emission wavelength of 520 nm. IC50 values were calculated from percentage of inhibition of ADAMTS-7 activity as a function of test compound concentration. IC50 values derived using functional ADAMTS-7 according to SEQ ID NO: 1 or SEQ ID NO: 2, respectively, were not distinguishable, both laying within the experimental error.

### Example 8: Expression Constructs for Production of Recombinant ADAMTS-12 Enzymes

Rat/human ADAMTS-12 chimera sequence (rPro-hCD (Rat 1-244/Human 241-543)-3×FLAG, SEQ ID 15) encoding rat pro-domain of ADAMTS-12 (amino acids 1-244 of rat sequence UniProt D3ZTJ3, which includes the signal peptide) and CD domain of human ADAMTS-12 (amino acids 241-543 of human sequence UniProt P58397) followed by 3×FLAG Tag was cloned into the mammalian pcDNA3.4 expression vector.

Rat/human ADAMTS-12 WT demonstrated a better expression profile compared to human ADAMTS-12 (1-543) WT with a human prodomain (FIG. 6). Optimization of the furin cleavage site (L237R) in the context of the human prodomain did not improve the yield of processed CD proteins compared to the rat/human ADAMTS-12 construct. Each ADAMTS-12 construct was mutated in parallel at the catalytic site with an E397Q substitution (EQ) with respect to SEQ ID NO: 15 resulting in increased protein yield similar to ADAMTS-7 catalytic mutations. Corresponding yield for the rat/human ADAMTS-12 EQ construct was also higher compared to ADAMTS-12 (1-543) EQ containing the human prodomain.

This expression construct allows production of recombinant ADAMTS-12 enzymes—either containing at least parts of both domains (e.g., prodomain plus catalytic domain as encoded, in which the prodomain can optionally be preceded by a signal peptide and/or the catalytic domain can optionally be followed by a disintegrin domain) or containing primarily the catalytic domain (e.g., catalytic domain as encoded, which can optionally be followed by a disintegrin domain).

### Example 9: Recombinant Production of Active ADAMTS-12 Enzyme

Expi293 cells (Life technologies, A14635) were grown and transfected in accordance to the manufacturer instruction. Briefly, the Expi 293 cells at the final cell density of 2.5×10⁶ cells/mL with >95% viability were transfected by the 1 mg/liter of vector plasmid DNA using Expifectamine transfection reagent (Life technologies, A14525). The overall purification scheme was similar to that used for ratADAMTS-7 (SEQ ID NO: 3).

Approximately 72 hours post transfection, Expi293 cell culture was centrifuged at 4000 rpm (˜3700 rcf) for 10 mins. Supernatant was collected and neutralized with 50 mM Tris pH 8.0, 5 mM CaCl₂, 10 μM ZnCl₂ before centrifuged again at 4000 rpm (˜3700rcf) for 10 min. Final supernatant was filtered through 0.22 μm filter.

The filtered supernatant was incubated overnight at 4° C. with anti-FLAG M2 affinity gel (Sigma-Aldrich A2220) equilibrated with buffer A (50 mM Tris pH 8.0, 300 mM NaCl, 10% glycerol, 5 mM CaCl₂, 10 μM ZnCl₂). The gel was collected and washed with 10 bed volumes of buffer A. The bound proteins were eluted by 100% buffer B (50 mM Tris pH 8.0, 300 mM NaCl, 10% glycerol, 5 mM CaCl₂, 10 μM ZnCl₂, 150 ng/μ1 FLAG peptide (Sigma-Aldrich F4799)). The collected fractions were analyzed on the SDS gel. The fractions containing ADAMTS-12 protein were combined and concentrated 10 times.

The concentrated material from the FLAG affinity purification was loaded onto superdex S200 (SEC) column equilibrated in column buffer: 20 mM Tris pH 8.0, 300 mM NaCl, 10% glycerol, 5 mM CaCl₂ and 10 μM ZnCl₂. The collected fractions were analyzed on the SDS gel. Fractions containing ADAMTS-12 protein were combined and concentrated to 0.5 mg/ml. Aliquoted proteins were flash frozen in liquid nitrogen and stored at −80° C.

### Example 10: Assay for ADAMTS-12 Enzymatic Activity and Testing of Inhibitory Compounds

Purified recombinant ADAMTS-12 was diluted in the reaction buffer (20 mM HEPES pH 8.0; 10 mM NaCl; 7.5 mM CaCl₂); 0,004% Brij; 7.5 μM ZnCl₂; 0.1% SmartBlock (Candor Bioscience 113125)) to the concentration of approximately 20 nM and 25 μl were transferred into each single well of 384-well white microtiter plate (Greiner Bio One 781075). 1 μl of the inhibitor compound solution (dissolved in DMSO, at the corresponding concentration) or pure DMSO as a control was added to the same wells. The enzymatic reaction was initiated by addition of 25 μl of 2 μM solution of the FRET substrate HiLyte Fluor 488-DELSSMVLELRGLRT-K(Q)<L520)E-NH2; (e.g., SEQ ID NO: 11) in the reaction buffer. It was surprisingly found that the same substrate could be used for the paralogs ADAMTS-7 and ADAMTS-12. The microtiter plate was incubated for 120 min at the temperature of 32° C. The increase of fluorescence intensity was measured in appropriate fluorescence plate reader (e.g. TECAN Ultra) using excitation wavelength of 485 nm and emission wavelength of 520 nm. IC50 values were calculated from percentage of inhibition of ADAMTS-12 activity as a function of test compound concentration.

### Example 11: Selectivity Assay for ADAMTS-7 and/or ADAMTS-12 Modulators

The respective enzyme (see Table 4 below) was diluted in reaction buffer (50 mM Tris, 2.5 μM ZnCl₂, 0.05% BSA, 0.001% Brij, pH 7.5 for ADAM17; 50 mM Tris 7.5, 150 mM NaCl, 10 mM CaCl₂, 0.05% Brij for all other enzymes) to the respective concentration and 25 μl were transferred into each well of a 384-well white microtiter plate (Greiner Bio One 781075). 1 μl test compound solution (dissolved in DMSO, at the corresponding concentration) or pure DMSO as a control was added per well. The enzymatic reaction was initiated by addition of 25 μl of the respective concentration of the respective FRET substrate (see Table 4 below) in reaction buffer. The microtiter plate was incubated for 120 min at the temperature of 32° C. The increase of fluorescence intensity was measured in appropriate fluorescence plate reader (e.g. TECAN Ultra) using the respective wavelengths for excitation and emission. IC50 values were calculated from percentage of inhibition of enzyme activity as a function of test compound concentration.

TABLE 4

| | | | | | | Source | Excita- | Emis- |
|---|---|---|---|---|---|---|---|---|
| | | | | | | Sub- | tion | sion |
| | Source | Conc. | | | Conc. | strate | Wave- | Wave- |
| Enzyme | Enzyme | Enzyme | | FRET substrate | Substrate | Jerini | length | length |
| ADAMTS | R&D 4307-AD | 50 | nM | Dabcyl-EEVKAKVQPY- | 1 μM | Peptide | 340 nm | 480 nm |
| 4 | | | | Glu(Edans)-NH2 | | Technol- | | |
| | | | | (e.g., SEQ ID NO: 14) | | ogies | | |
| ADAMTS | R&D 2198-AD | 100 | nM | Dabcyl-EEVKAKVQPY- | 25 μM | Jerini | 340 nm | 480 nm |
| 5 | | | | Glu(Edans)-NH2 | | Peptide | | |
| | | | | (e.g., SEQ ID NO: 14) | | Technol- | | |
| | | | | | | ogies | | |
| MMP12 | R&D 917- | 1 | nM | Mca-PLGLEEA-Dap(Dnp)- | 10 μM | Bachem | 325 nm | 393 nm |
| | MPB, activated | | | NH2 | | M-2670 | | |
| | according to | | | (e.g., SEQ ID NO: 17) | | | | |
| | manufacturers | | | | | | | |
| | instruction | | | | | | | |
| MMP15 | R&D 916-MP, | 6 | nM | Mca-KPLGL-Dpa-AR-NH2 | 20 μM | R&D | 320 nm | 405 nm |
| | activated | | | (e.g., SEQ ID NO: 18) | | ES010 | | |
| | according to | | | (Neumann, U. etal., 2004, | | | | |
| | manufacturers | | | Anal. Biochem. 328:166- | | | | |
| | instruction | | | 173) | | | | |
| MMP2 | R&D 902-MP, | 0.06 | nM | Mca-PLGL-Dpa-AR-NH2 | 20 μM | R&D | 320 nm | 405 nm |
| | activated | | | (e.g., SEQ ID NO: 19) | | ES001 | | |
| | according to | | | | | | | |
| | manufacturers | | | | | | | |
| | instruction | | | | | | | |
| ADAM17 | R&D 930-ADB | 20 | nM | Mca-PLAQAV-Dap(Dnp)- | 20 μM | Bachem | 320 nm | 405 nm |
| | | | | RSSSR-NH2 | | M-2255 | | |
| | | | | (e.g., SEQ ID NO: 20) | | | | |

Table 4. Assay conditions for the evaluation of the selectivity of ADAMTS-7 modulators. Abbreviations: Dabcyl: 4-((4-(dimethylamino)phenyl)azo)benzoic acid; Glu(Edans): L-glutamic acid-γ-[2-(1-sulfonyl-5-naphthyl)-aminoethylamide]; Mca: (7-Methoxycoumarin-4-yl)acetyl; Dap(Dnp): (2S)-3-(2,4-dinitroanilino)-2-(9H-fluoren-9-ylmethoxycarbonylamino)propanoic acid; Dpa: N-3-(2, 4-Dinitrophenyl)-L-2,3-diaminopropionyl.

### Example 12: Modulators of ADAMTS

The assays to determine the IC50 for ADAMTS inhibition were performed as described earlier herein.

TABLE 5

| Example | ADAMTS-7-IC₅₀ | ADAMTS-4-IC₅₀ |
|---|---|---|
| Nr. | [nM] | [nM] |
| 12.1 | 140 | 4750 |
| 12.2 | 355 | 24000 |
| 12.3 | 550 | 50000 |
| 12.4 | 480 | 50000 |
| 12.5 | 52 | 4690 |

### Example 12.1

### N-{[(4R)-4-cyclopropyl-2,5-dioxoimidazolidin-4-yl]methyl}-2-(5-fluoropyridin-2-yl)-2H-1,2,3-triazole-4-carboxamide

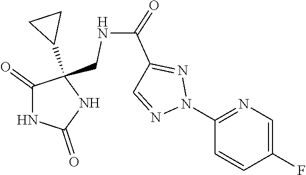 [SMILES: O=C1NC(=O)[C@](CNC(=O)c2cnn(-c3ccc(F)cn3)n2)(C2CC2)N1]

The compound was synthesized according to the General Method A using 111 mg (0.53 mmol) of 2-(5-fluoropyridin-2-yl)-2H-1,2,3-triazole-4-carboxylic acid, 100 mg (0.48 mmol) of (5R)-5-(aminomethyl)-5-cyclopropylimidazolidine-2,4-dione hydrochloride, 121 mg (0.63 mmol) of 1-(3-dimethylaminopropyl)-3-ethylcarbodiimide hydrochloride, 97 mg (0.63 mmol) of 1-hydroxybenzotriazole and 0.24 mL (1.40 mmol) of N,N-diisopropylethylamine in 3 mL of N,N-dimethylformamide. The crude product obtained from the workup was purified by reversed-phase chromatography using a gradient from 95% 65 mM ammonium acetate:acetonitrile 90:10/5% acetonitrile:methanol 1:1 to 63% 65 mM ammonium acetate:acetonitrile 90:10/37% acetonitrile:methanol 1:1 to give 5 mg (3% yield, 99% purity) of the pure compound as an off-white powder.

¹H-NMR (300 MHz, DMSO-d₆): δ [ppm]=0.07-0.20 (m, 1H), 0.29-0.49 (m, 3H), 1.10-1.22 (m, 1H), 3.71 (d, 2H), 7.61 (s, 1H), 7.99-8.19 (m, 2H), 8.48-8.59 (m, 2H), 8.65 (s, 1H), 10.65 (s, 1H).

LC-MS (Method 1): Rt=1.908 min. MS (Mass method 1): m/z=360 (M+H)⁺

### Example 12.2

### N-{[(4R)-4-cyclopropyl-2,5-dioxoimidazolidin-4-yl]methyl}-2-(pyrazin-2-yl)-2H-1,2,3-triazole-4-carboxamide

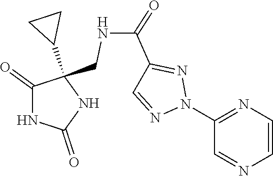 [SMILES: O=C1NC(=O)[C@](CNC(=O)c2cnn(-c3cnccn3)n2)(C2CC2)N1]

The compound was synthesized according to the General Method A using 60 mg (0.31 mmol) of 2-(pyrazin-2-yl)-2H-1,2,3-triazole-4-carboxylic acid, 71 mg (0.34 mmol) of (5R)-5-(aminomethyl)-5-cyclopropylimidazolidine-2,4-dione hydrochloride, 78 mg (0.41 mmol) of 1-(3-dimethylaminopropyl)-3-ethylcarbodiimide hydrochloride, 62 mg (0.41 mmol) of 1-hydroxybenzotriazole and 0.15 mL (0.88 mmol) of N,N-diisopropylethylamine in 3 mL of N,N-dimethylformamide. The crude product obtained from the workup was precipitated with diethyl ether and heptane to give 11 mg (10% yield, 99% purity) of the pure compound as an off-white powder.

¹H-NMR (300 MHz, DMSO-d₆): δ [ppm]=0.07-0.19 (m, 1H), 0.24-0.46 (m, 3H), 1.18-1.21 (m, 1H), 3.68 (ddd, 2H), 7.39 (bp, 1H), 8.57-8.66 (m, 2H), 8.72 (s, 1H), 8.84 (s, 1H), 9.38 (s, 1H), 10.65 (bp, 1H).

LC-MS (Method 1): Rt=1.580 min. MS (Mass method 1): m/z=343 (M+H)⁺

### Example 12.3

### N-{[(4R)-4-cyclopropyl-2,5-dioxoimidazolidin-4-yl]methyl}-3-[1-(cyclopropylmethyl)-1H-pyrazol-4-yl]-1,2-oxazole-5-carboxamide

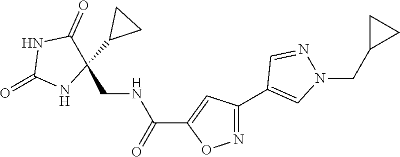 [SMILES: O=C1NC(=O)[C@](CNC(=O)c2cc(-c3cnn(CC4CC4)c3)no2)(C2CC2)N1]

929 mg (2.44 mmol) of HATU were added to a solution of 380 mg (1.63 mmol) of 3-[1-(cyclopropylmethyl)-1H-pyrazol-4-yl]-1,2-oxazole-5-carboxylic acid, 335 mg (1.63 mmol) of (5R)-5-(aminomethyl)-5-cyclopropylimidazolidine-2,4-dione hydrochloride, 2.40 mL (2.40 mmol) of 1M 1-hydroxy-7-azabenzotriazole in N,N-dimethylacetamide and 0.85 mL (4.90 mmol) of N,N-diisopropylethylamine in 5 mL of N,N-dimethylformamide and the reaction was allowed to stir at room temperature for 16 hours. The solvent was evaporated and the residue was diluted with dichloromethane, sequentially washed with an aqueous solution of sodium hydrogencarbonate (aq.) and 2N hydrochloric acid (aq.). The organic layer was dried over magnesium sulfate, filtered and concentrated to afford a residue, which was further purified by flash chromatography on silica gel eluting with dichloromethane/methanol mixtures to give 20 mg (3% yield, 99% purity) of the product as a white solid.

1H-NMR (300 MHz, DMSO-d₆): δ [ppm]=0.07-0.19 (m, 1H), 0.28-0.50 (m, 5H), 0.51-0.67 (m, 2H), 1.09-1.19 (m, 1H), 1.23-1.34 (m, 1H), 3.66 (ddd, 2H), 4.03 (d, 2H), 7.39 (s, 1H), 7.94 (s, 1H), 8.38 (s, 1H), 8.87 (t, 1H), 10.64 (s, 1H).

LC-MS (Method 1): Rt=2.166 min. MS (Mass method 1): m/z=385 (M+H)⁺

### Example 12.4

### rac-{[4-tert-butyl-2,5-dioxoimidazolidin-4-yl]methyl}-2-phenyl-1,3-oxazole-5-carboxamide

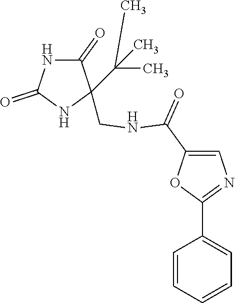 [SMILES: CC(C)(C)C1(CNC(=O)c2cnc(-c3ccccc3)o2)NC(=O)NC1=O]

226 mg (0.59 mmol) of HATU were added to a solution of 75 mg (0.39 mmol) of 2-phenyl-1,3-oxazole-5-carboxylic acid, 73 mg (0.39 mmol) of rac-5-(aminomethyl)-5-tert-butylimidazolidine-2,4-dione, 0.59 mL (0.59 mmol) of 1M 1-hydroxy-7-azabenzotriazole in N,N-dimethylacetamide and 0.17 mL, (1.20 mmol) of triethylamine in 5 mL of N,N-dimethylformamide and the reaction was allowed to stir at room temperature for 1 hour. The reaction was quenched with methanol and the solvent was removed in vacuo. The corresponding residue was diluted with ethyl acetate and sequentially washed with 1N hydrochloric acid (aq.), saturated sodium hydrogencarbonate (aq.) and brine. The organic layer was dried over magnesium sulfate, filtered, concentrated and purified by flash chromatography on silica gel eluting with dichloromethane and methanol to give 86 mg (58% yield, 95% purity) of the racemic product as a white powder.

1H-NMR (300 MHz, DMSO-d₆): δ [ppm]=1.03 (s, 9H), 3.71 (ddd, 2H), 7.53-7.65 (m, 4H), 7.90 (s, 1H), 8.06-8.15 (m, 2H), 8.61 (t, 1H), 10.61 (bp, 1H).

LC-MS (Method 1): Rt=2.385 min. MS (Mass method 1): m/z=357 (M+H)⁺

### Example 12.5

### N-{[(4R)-4-cyclopropyl-2,5-dioxoimidazolidin-4-yl]methyl}-3-(1-methyl-1H-pyrazol-4-yl)-1,2-oxazole-5-carboxamide

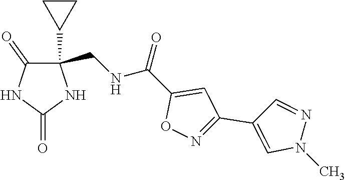 [SMILES: Cn1cc(-c2cc(C(=O)NC[C@@]3(C4CC4)NC(=O)NC3=O)on2)cn1]

To a solution of (5R)-5-(aminomethyl)-5-cyclopropylimidazolidine-2,4-dione hydrochloride (60.0 mg, 292 μmol) and 3-(1-methyl-1H-pyrazol-4-yl)-1,2-oxazole-5-carboxylic acid (62.0 mg, 321 μmol) in N,N-dimethylformamide (1 ml) was added 1-ethyl-3-(3-dimethylaminopropyl)carbodiimide hydrochloride (72.7 mg, 379 μmol), (1.3 eq) 1-hydroxybenzotriazole hydrate (58.1 mg, 379 μmol) and N,N-diisopropylethylamine (140 μl, 817 μmol). The reaction mixture was stirred at RT for 16 h. The reaction was partitioned between ethyl acetate and a saturated solution of NaHCO₃. The water layer was washed with ethyl acetate, dried over magnesium sulfate, filtered and concentrated. Acetonitrile was added to the crude and the resulting precipitate was filtered off and dried in vacuo to afford the product. The obtained amount of product was 35 mg (100% purity, 35% of theory).

LC/MS (Method 7): Rt=0.84 min, MS (ESIpos): m/z=345 [M+H]⁺

¹H-NMR (500 MHz, DMSO-d₆) δ[ppm]: 0.116 (0.71), 0.125 (1.04), 0.135 (1.08), 0.144 (0.82), 0.333 (0.73), 0.345 (0.93), 0.354 (0.89), 0.361 (0.58), 0.370 (0.42), 0.422 (0.75), 0.439 (1.51), 0.450 (1.55), 0.455 (1.57), 0.466 (1.24), 0.476 (0.64), 1.135 (0.76), 1.140 (0.83), 1.151 (1.30), 1.161 (0.76), 1.167 (0.65), 3.600 (0.74), 3.612 (0.82), 3.628 (1.62), 3.640 (1.45), 3.668 (1.50), 3.682 (1.57), 3.695 (0.79), 3.709 (0.72), 3.904 (16.00), 7.373 (6.69), 7.599 (3.81), 7.932 (5.28), 8.309 (5.13), 8.872 (0.96), 8.884 (1.77), 8.897 (0.89), 10.651 (2.32).

### Example 13: Protease Activity is Essential for the Pro-Atherogenic Effects of ADAMTS7

The secreted metalloproteinase ADAMTS7 has been associated with coronary artery disease (CAD) and loss of ADAMTS7 is protective for atherosclerosis, yet a connection between enzymatic function and CAD risk remains unresolved. We therefore generated a catalytic mutant mouse allele and compared it to the Adamts7 knockout. Using two models of atherosclerosis, we found that a reduction of Adamts7 dosage or catalytic function resulted in prevention of atherosclerosis. We then characterized a coding variant, Ser214Pro, associated with CAD risk. This variant had a hypomorphic effect on Adamts7 secretion and migration of vascular smooth muscle cells, findings consistent with our mouse studies. In aggregate, we provide compelling evidence that ADAMTS7 is an attractive therapeutic target for CAD.

Coronary artery disease is a major cause of mortality worldwide and newly identified genetic risk factors are currently being sought for therapeutic development (Khera and Kathiresan (2017) Nat Rev Genet 18: 331-344). Multiple genome wide association studies (GWAS) have identified variants within ADAMTS7 (Reilly et al. (2011) Lancet 377: 383-392; Schunkert et al. (2011) Nat Genet 43: 333-338) and in the flanking intergenic regions associated with CAD (C4D Genetics Consortium (2011) Nat Genet 43: 339-344; Dichgans et al. (2014) Stroke 45: 24-36). ADAMTS7 risk variants are associated with a high-risk plaque phenotype and an increase in secondary cardiovascular events (Pereira et al. (2016) Physiol Genomics 48: 810-815; Chan et al. (2017) J Am Heart Assoc 6(11): e006928; Bengtsson et al. (2017) Sci Rep 7: 3753; Li et al. (2019) Medicine (Baltimore) 98: e17438). Initial characterization of a coding variant rs3825807 (Ser214Pro) have shown effects for zymogen processing consistent with beneficial effects from less ADAMTS7 function (Pu et al. (2013) Am J Hum Genet 92: 366-374). A recent study demonstrated that Adamts7 deficiency in hyperlipidemic ApoE KO or Ldlr KO background mice results in reduced atherosclerotic lesion formation, suggesting that ADAMTS7 is pro-atherogenic (Bauer et al. (2015) Circulation 131: 1202-1213). Further studies performed in rodent vascular injury models support a role for ADAMTS7 loss of function as protective against vascular smooth muscle cell migration and proliferation in neointima formation (Bauer et al. (2015) Circulation 131: 1202-1213; Wang et al. (2009) Circ Res 104: 688-698; Kessler et al. (2015) Circulation 131: 1191-1201; Zhang et al. (2015) Sci China Life Sci 58: 674-681). Despite all of this genetic evidence, it is presently unclear if the catalytic function of ADAMTS7 directly contributes to the atherosclerosis phenotype.

ADAMTS7 (a disintegrin and metalloproteinase with thrombospondin motifs 7) belongs to a family of secreted zinc metalloproteinases containing elaborate carboxyl-terminal ancillary domains (Mead and Apte (2018) Matrix Biol 71-72: 225-239). The catalytic domain of all 19 ADAMTS enzymes is preceded by a signal peptide and prodomain that serves as a chaperone and contains a “cysteine switch” to engage with the catalytic zinc to maintain enzyme latency (Cerda-Costa and Gomis-Ruth (2014) Protein Sci 23: 123-144). The ADAMTS catalytic domain is followed by a set of disintegrin, thrombospondin, cysteine-rich and spacer domains thought to confer substrate specificity and localization (Kelwick et al. (2015) Genome Biol 16: 113). ADAMTS7 contains a total of eight thrombospondin repeats and a highly glycosylated mucin domain with a chondroitin sulfate GAG attachment site rendering ADAMTS7 as both an enzyme and a proteoglycan (Somerville et al. (2004) J Biol Chem 279: 35159-35175). It is notable that there are seven ADAMTS-like (ADAMTSL) proteins which lack the catalytic domain; however, ADAMTSL loss-of-function of mutations are associated with diseases, indicating a role for the ancillary domains in matricellular biology separate from catalytic function (Mead and Apte (2018) Matrix Biol 71-72: 225-239). ADAMTS7, and its close paralog ADAMTS12, are defined as cartilage oligomeric protein (COMP) proteases, capable of associating with COMP via their ancillary carboxyl-terminal domains (Liu et al. (2006) FASEB J20: 988-990; Liu et al. (2006) J Biol Chem 281: 15800-15808). Regulated cleavage of COMP or other extracellular matrix (ECM) proteins modulates VSMC migration (Pu et al. (2013) Am J Hum Genet 92: 366-374; Riessen et al. (2001) Arterioscler Thromb Vasc Biol 21: 47-54). However, a role for the auxiliary domains of ADAMTS7 on VSMC migration and atherogenesis remains a distinct possibility independent from the enzymatic function.

In the current work, we sought to address two primary questions. First, is the catalytic activity of ADAMTS7 essential for the pro-atherogenic effects? And second, could the atherosclerosis protective effects of ADAMTS7 loss be mediated by a known coding variant associated with CAD risk?

### Results

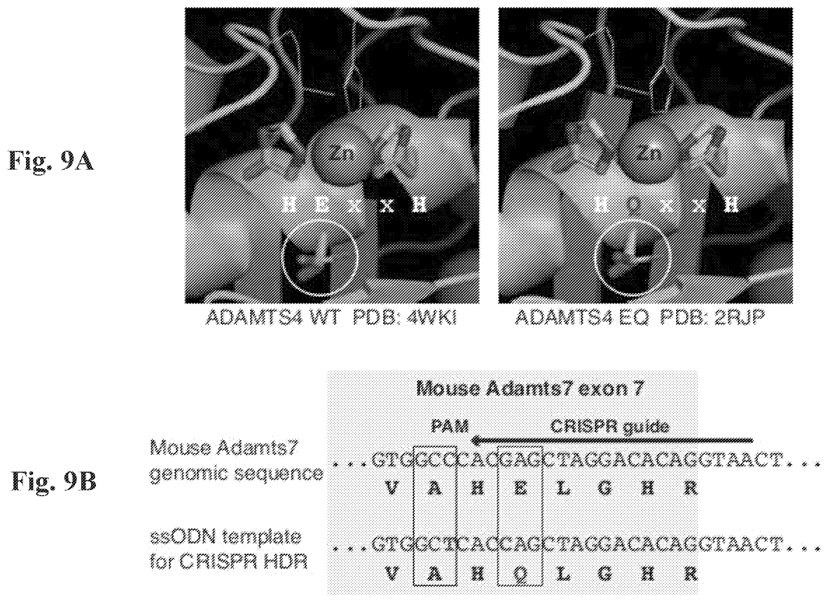

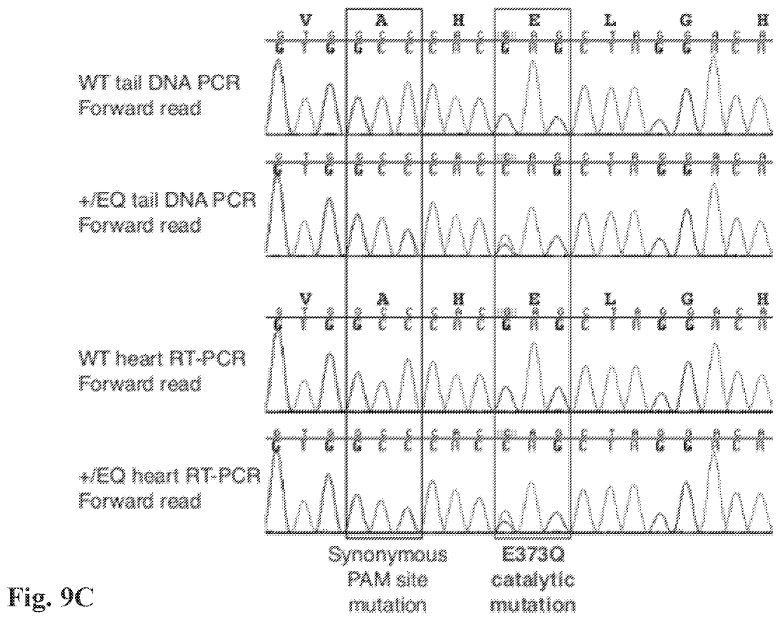

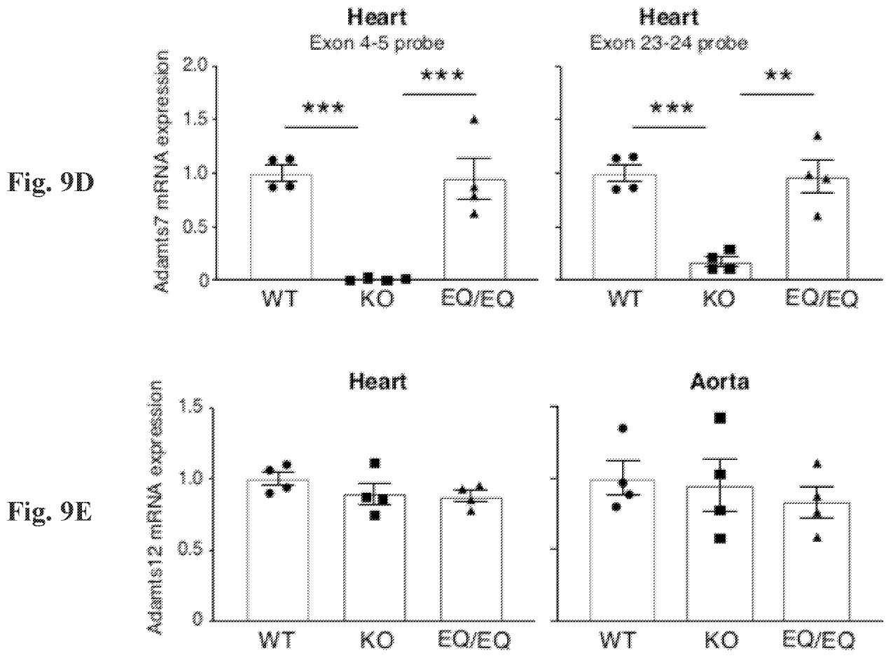

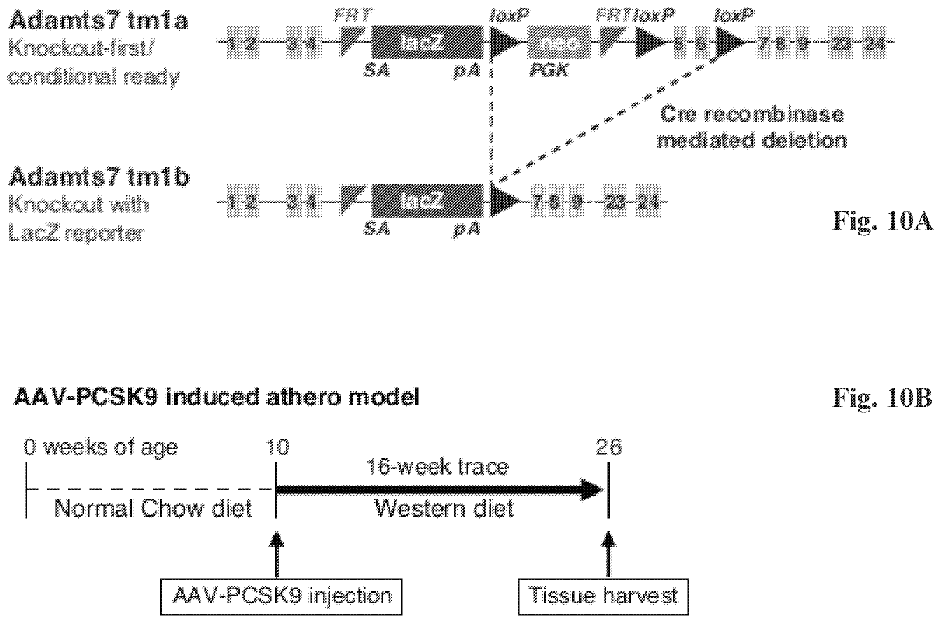

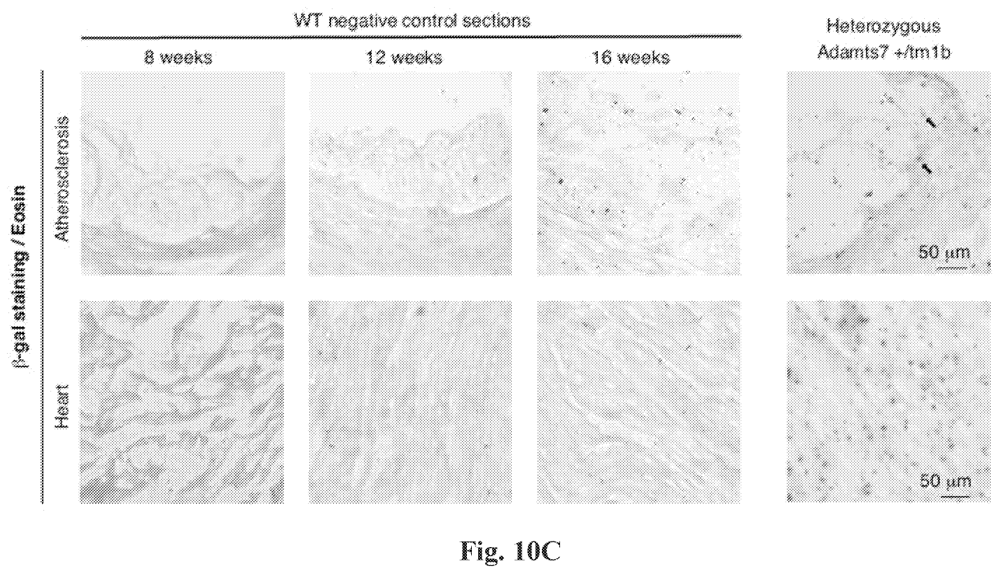

Genetic inhibition of ADAMTS7 catalytic function prevents atherosclerosis To determine if ADAMTS7 catalytic activity is essential for the phenotypic effects, we generated a catalytic mutant mouse and compared it to the loss of function allele. To eliminate catalytic function, we employed a glutamate to glutamine substitution to preserve the helical structure of the HExxH motif while preventing the generation of a reactive nucleophile at the zinc metalloprotease active site (Cerda-Costa and Gomis-Ruth (2014) Protein Sci 23: 123-144) (FIG. 9A). The conserved metalloproteinase HExxH motif is present in mouse ADAMTS7 and the corresponding mouse ADAMTS7 Glu373Gln (E373Q) mutation (CAG to CAC) was generated using CRISPR-HDR (FIG. 9B). From this approach, the Cas9 PAM site located in Ala371 (GCC) was also altered synonymously to prevent additional editing (GCT) resulting in two nucleotide positions changed in the E373Q catalytic mutant allele. We examined Adamts7+/E373Q heterozygotes and observed no evidence of allelic expression imbalance from these modified positions (FIG. 9C). To generate a knockout allele, we obtained the publicly available mouse Admats7 tm1a “knockout-first/conditional ready” allele which carries gene-trap LacZ reporter inserted into intron 4 ahead of loxP flanked exons 5 and 6. This allele was bred to EIIa-Cre mice for germline deletion of the loxP flanked exons encoding part of the catalytic domain to generate the Adamts7 tm1b knockout allele used in our study (FIG. 10A). To test for Adamts7 expression in the heart using our homozygous mouse alleles, we used two probe sets located at the exon 4-5 boundary (absent in the tm1b allele) and at the exon 23-24 boundary of the last intron. As expected, no detectable signal was present from exon 4-5 probe in the Adamts7 KO (tm1b homozygotes). Residual signal in the KO was present with the exon 23-24 probe, similar to what was observed in Adamts7 tm1a homozygous knockouts (Bauer et al. (2015) Circulation 131: 1202-1213). No difference in Adamts7 mRNA expression levels was observed between WT and E373Q/E373Q mice for either probe set (FIG. 9D).

ADAMTS12 is known to share the same domain organization as ADAMTS7 and the two paralogs likely evolved from a gene duplication event (Somerville et al. (2004) J Biol Chem 279: 35159-35175; Cal et al. (2001) J Biol Chem 276: 17932-17940). A recent paper reported compensatory regulation between Adamts7 and Adamts12 transcription levels, notably with upregulation of Adamts12 in the tendon from Adamts7 KO mice (Mead et al. (2018) JCI Insight 3(7): e92941). Therefore, we evaluated Adamts12 mRNA expression in the heart and aorta from WT, Adamts7 KO and E373Q/E373Q mice. We found no evidence of compensatory regulation of Adamts12 in these tissues (FIG. 9E). Using our characterized Adamts7 knockout and catalytic mutant alleles, we examined the development of atherosclerosis in two different atherogenic backgrounds.

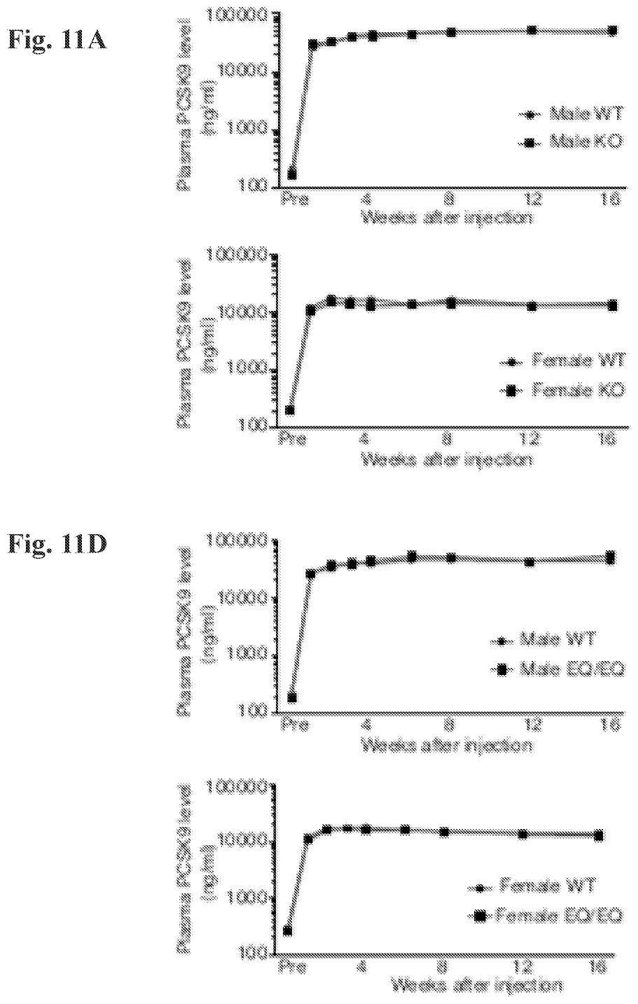

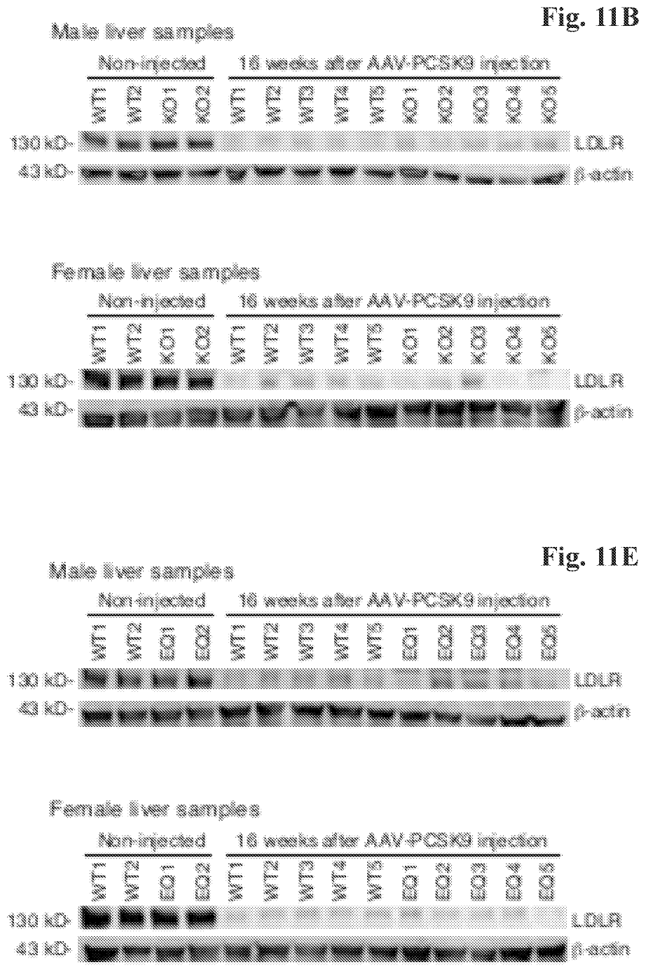

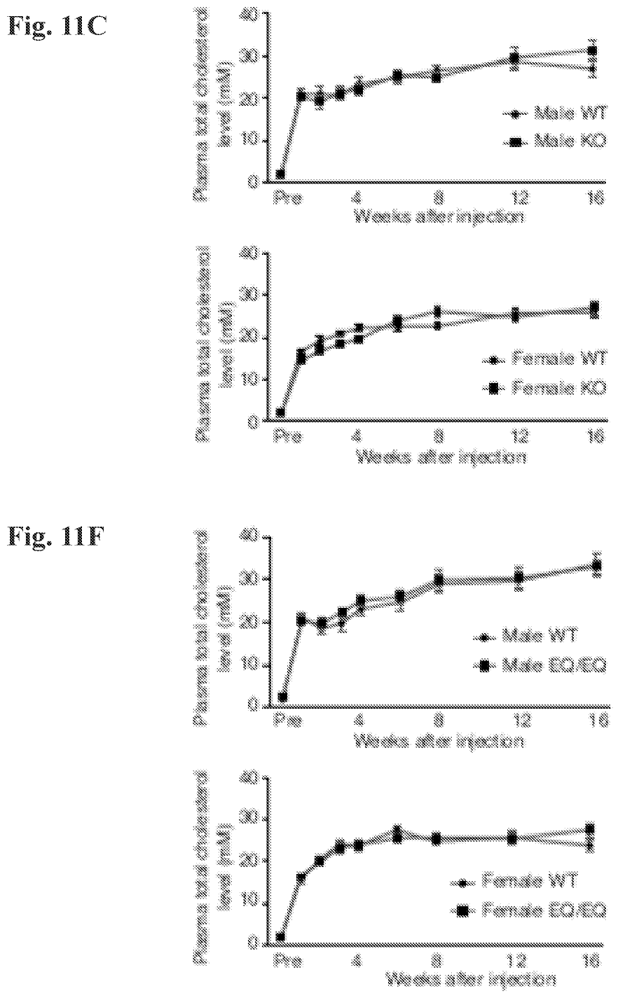

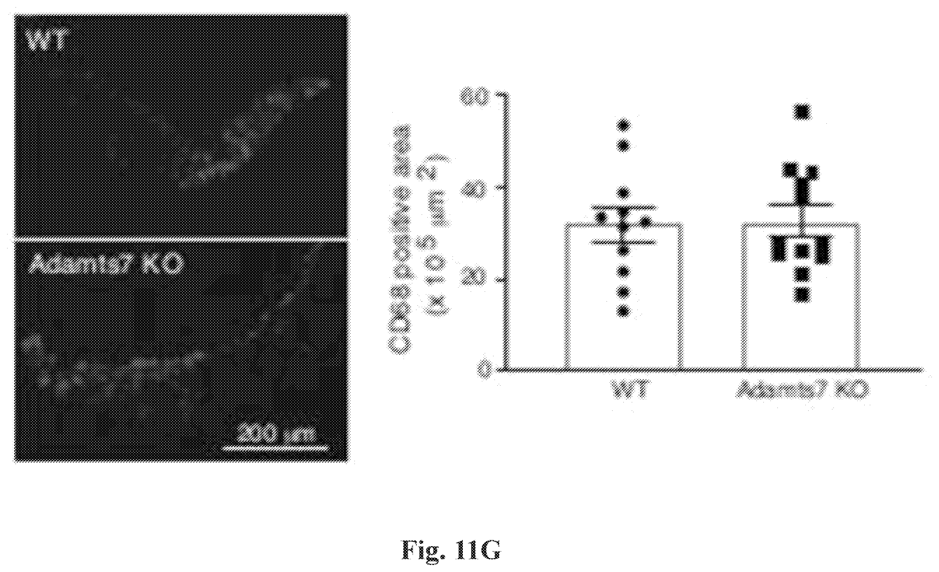

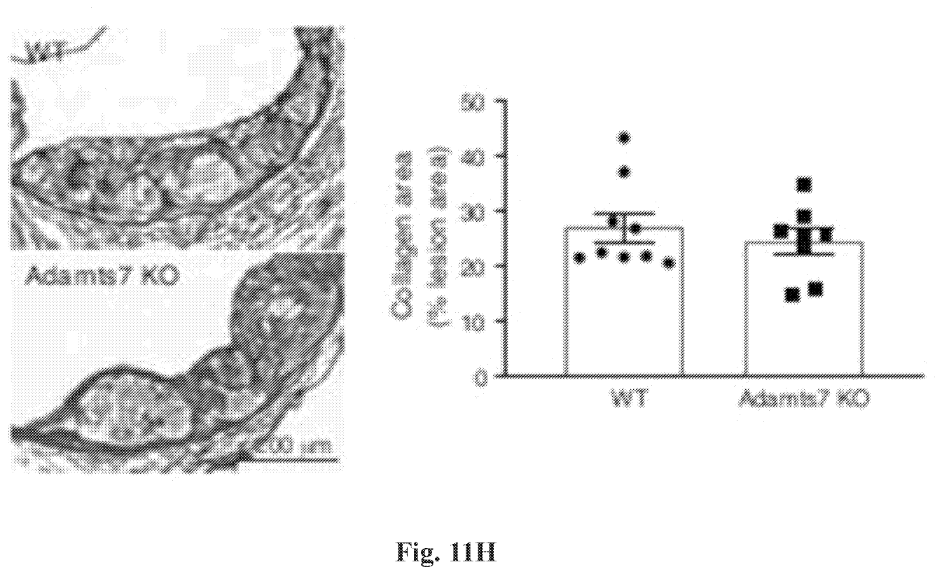

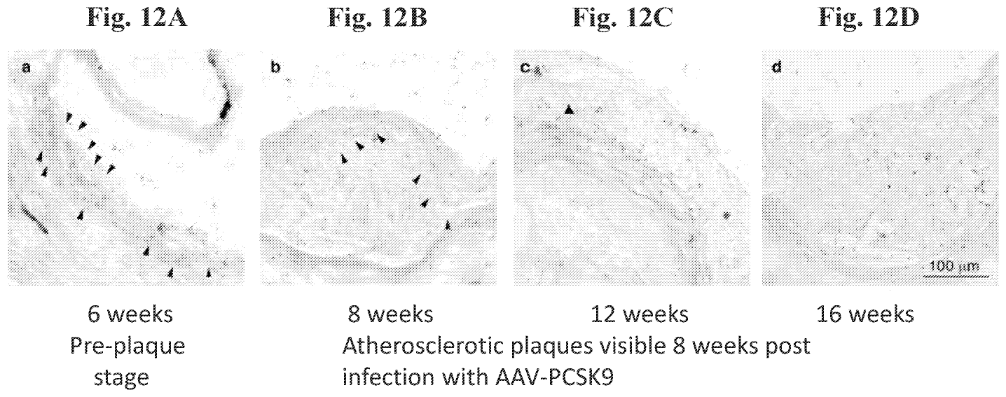

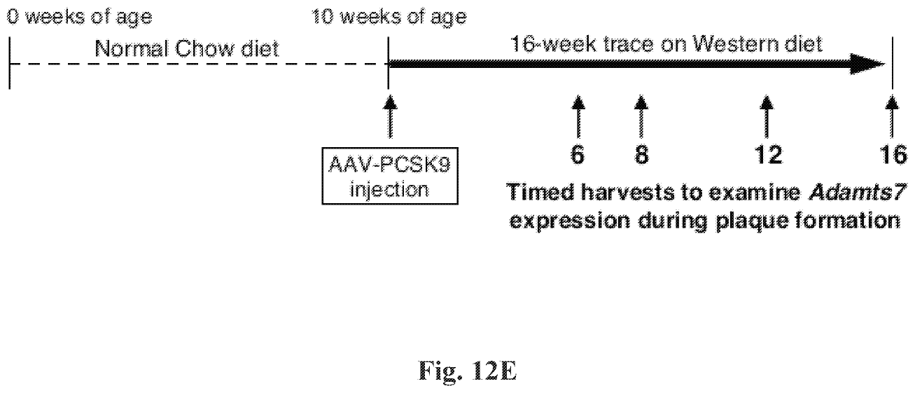

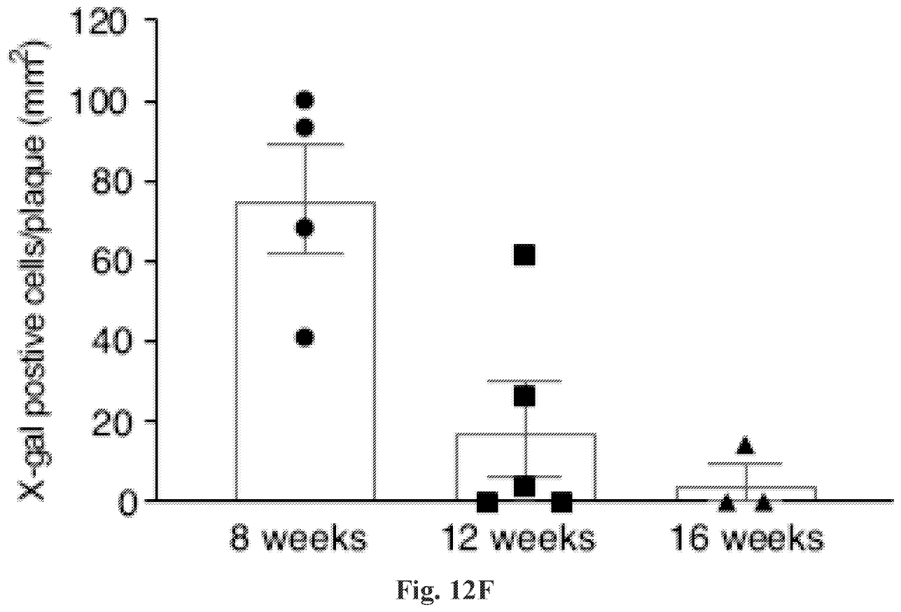

In the first model, adult mice were injected with a gain of function mutant form of PCSK9 (AAV8-mPCSK9 D377Y) and placed on a high fat Western diet for 16 weeks to induce atherosclerosis (Bjorklund et al. (2014) Circ Res 114: 1684-1689) (FIG. 10B). Following a single injection, plasma PCSK9 levels reached peak expression within one week and persisted for 16 weeks during the atherogenesis study (FIG. 11A). Plasma total cholesterol levels were >15 mM within one week and were persistently high, consistent with the significant reduction of LDLR in the liver at week 16 (FIG. 11B and FIG. 11C). We could measure atherosclerotic plaques build-up as early as 8 weeks post injection and continued to accumulate. To examine the expression of ADAMTS7 throughout development of atherosclerosis, we utilized the LacZ reporter from heterozygous Adamts7+/tm1b mice. X-gal staining was evident 6 weeks post injection before plaque formation and present in early plaques at 8 weeks post injection (FIG. 12A and FIG. 12B). Fewer X-gal positive cells were observed at 12 and 16 weeks post injection, consistent with previous reports of transient ADAMTS7-LacZ expression during atherogenesis (Bauer et al. (2015) Circulation 131: 1202-1213) (FIG. 12C-FIG. 12E). No X-gal staining was detected in littermate WT control atherosclerotic plaques or in the heart (FIG. 10C).

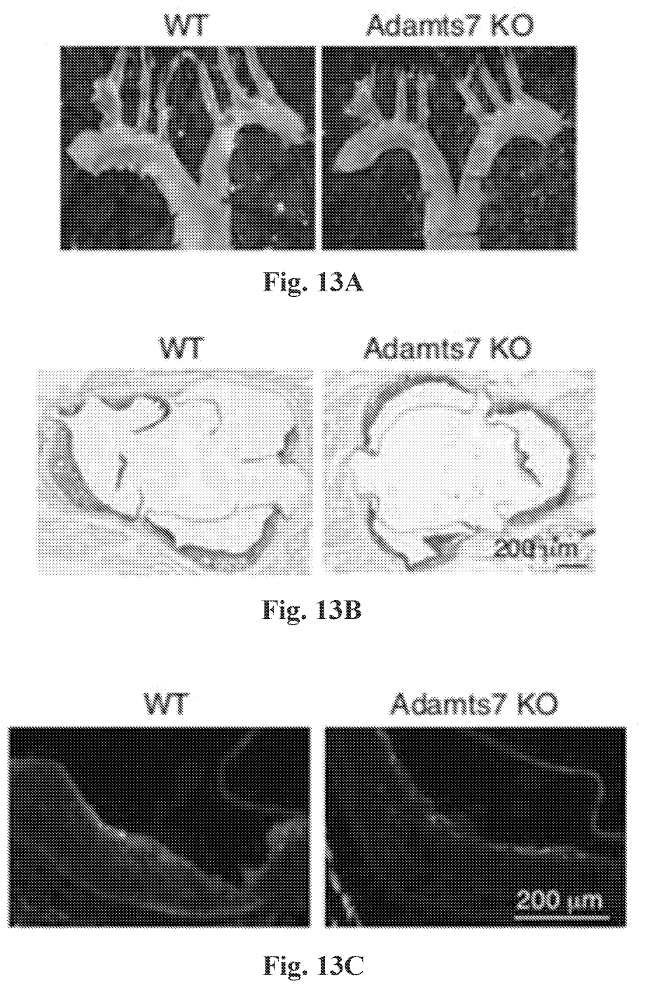

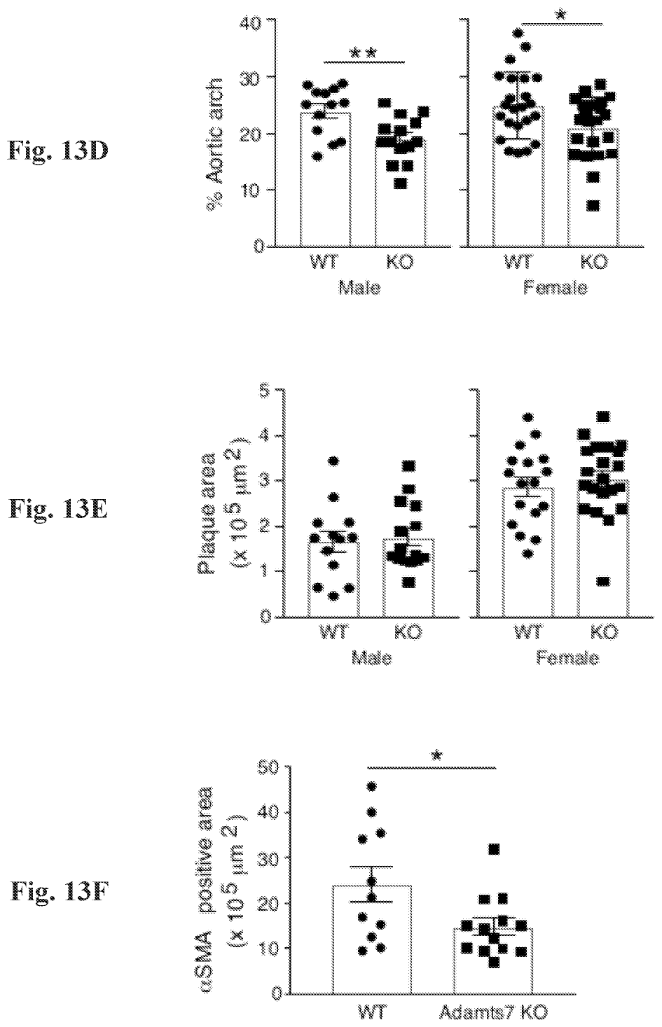

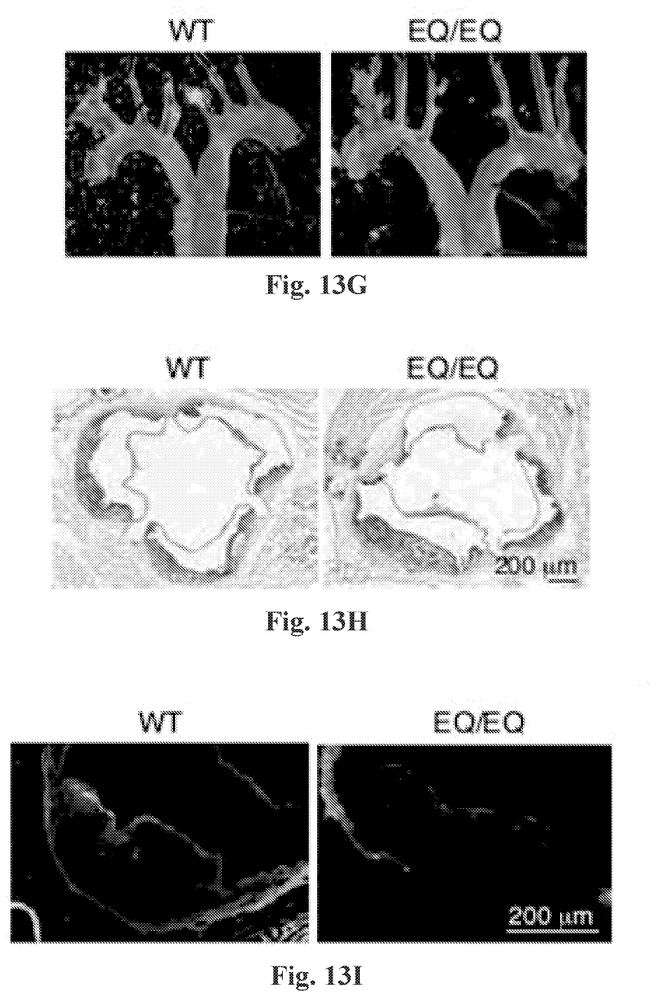

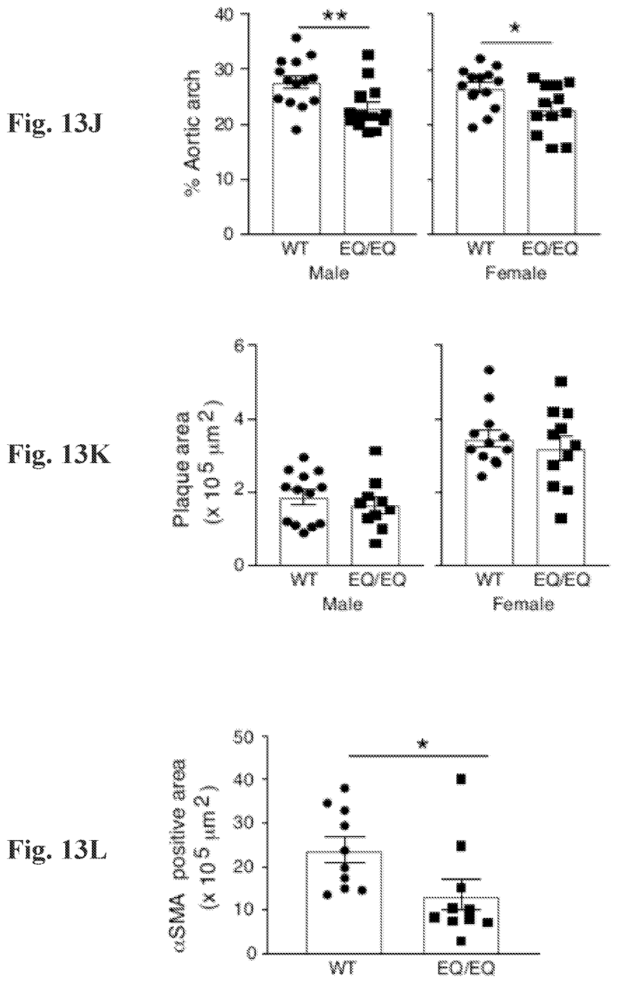

To confirm the effect of Adamts7 deficiency on the development of atherosclerosis, we examined male and female Adamts7 KO mice compared to littermate controls. Adamts7 KO mice showed significantly decreased lesion formation in en face analysis of the aortic arch region (mean±SEM for all en face results: control littermates 23.9±1.2% versus Adamts7 KO mice 19.0±1.1%; P<0.05 [male] and control littermates 25.0±1.2% versus Adamts7 KO mice 21.1±1.1%; P<0.05 [female]; FIG. 13A and FIG. 13D). From the same mice, we harvested and sectioned aortic roots to quantitatively assess plaque progression and composition. There was no significant difference in plaque burden in the aortic root between 2 groups (FIG. 13B and FIG. 13E). By the immunohistochemical analysis of aortic root plaques composition, we observed a significant reduction in SMC content in Adamts7 KO mice compared to control littermates (FIG. 13C and FIG. 13F). Macrophage accumulation and collagen content in the aortic plaque were not affected by Adamts7 deficiency (FIG. 11A-FIG. 1111).

Next, we examined male and female Adamts7 E373Q/E373Q homozygotes compared to littermate controls. Adamts7 E373Q/E373Q mice showed significantly decreased lesion formation in en face analysis of aortic arch (control littermates 27.7±1.2% versus E373Q/E373Q mice 22.9±1.2%; P<0.05 [male] and control littermates 26.7±1.0% versus E373Q/E373Q mice 22.8±1.3%; P<0.05 [female]; FIG. 13G and FIG. 13J). In parallel with the en face analysis, cross-sectional analysis of aortic roots was performed. There was no significant difference in plaque burden in the aortic root between 2 groups (FIG. 13H and FIG. 13K). Consistent with the reduced SMC content in the aortic plaque from Adamts7 KO mice, a significant reduction in SMC content was observed in Adamts7 E373Q/E373Q mice (FIG. 13I and FIG. 13L). Collectively, these results show that atherosclerosis-related phenotype of the catalytically inactive ADAMTS7 mice mirrors the atherosclerosis phenotype of Adamts7 KO mice.

ADAMTS7 Dosage and Catalytic Activity Controls Atherogenesis

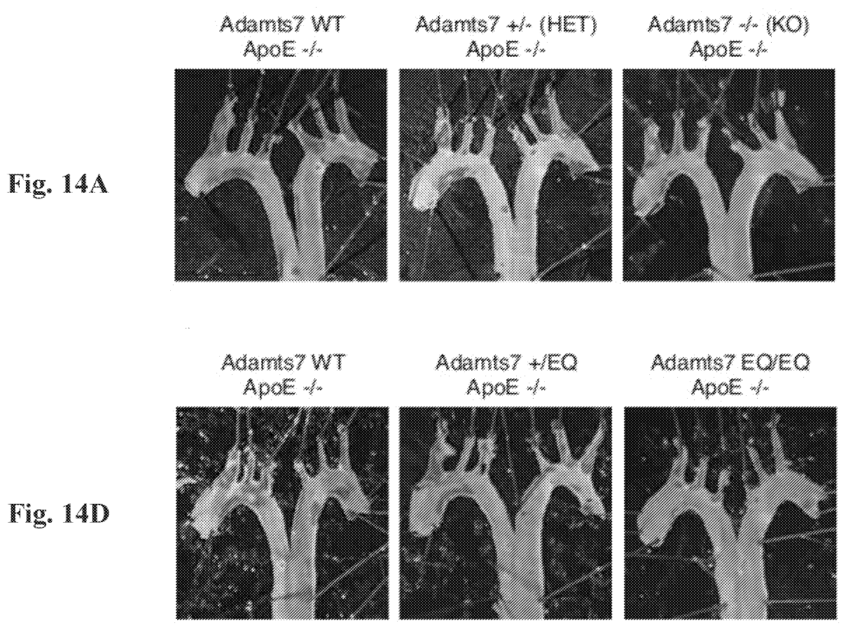

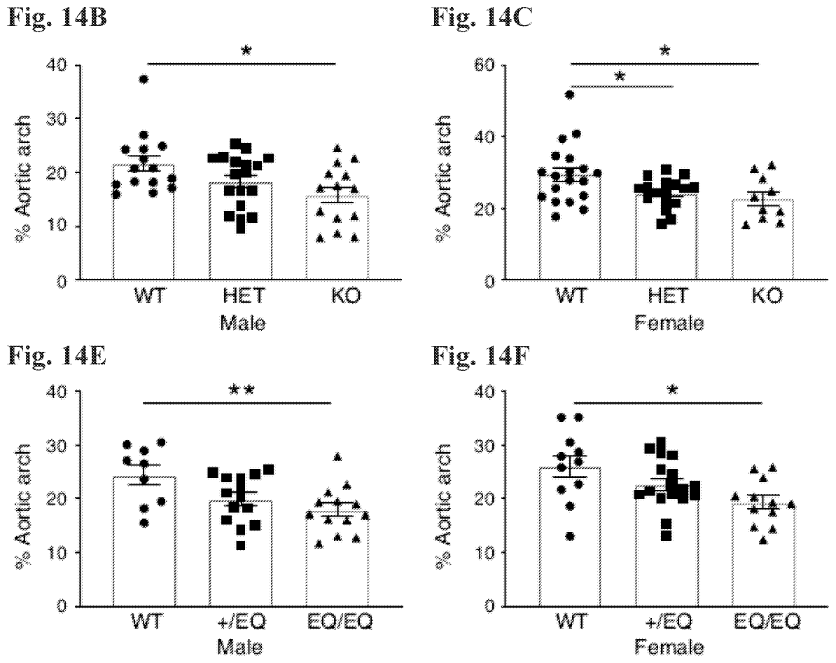

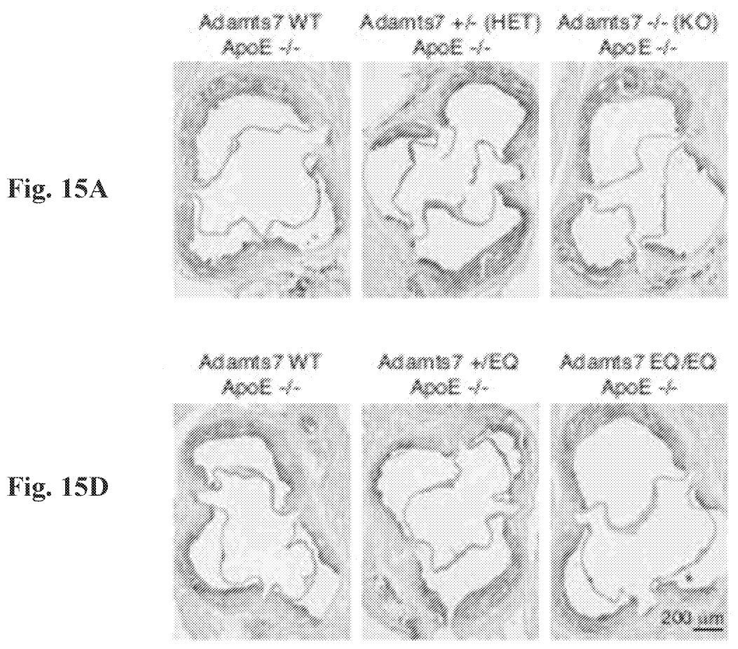

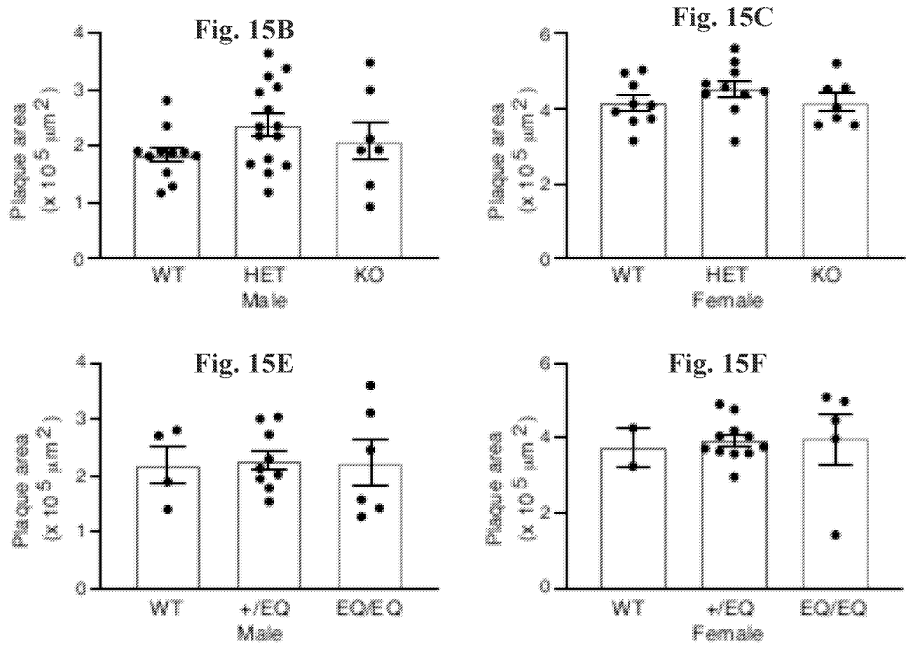

In the second mouse model, we backcrossed the Adamts7 alleles to the atherogenic ApoE KO background. To assess the dosage effects of Adamts7 loss of function, we examined WT, heterozygous and homozygous KO mice exposed to a high-fat Western diet for 10 weeks. On this background, we observed a decrease in lesion formation in the aortic arch region between WT and Adamts7 KO mice similar to previous reports (Bauer et al. (2015) Circulation 131: 1202-1213) (FIG. 14A). Quantitation of the en face results showed a significant reduction between WT and KO (control littermates 21.6±1.4% versus Adamts7 KO mice 15.8±1.5%; P<0.05 [male] and control littermates 29.5±1.9% versus Adamts7 KO mice 22.7±2.0%; P<0.05 [female]; FIG. 14B and FIG. 14C). A comparison between WT and heterozygotes missing one functional Adamts7 allele displayed a similar trend suggesting benefits from 50% loss of function (control littermates 21.6±1.4% versus Adamts7+/tm1b mice 18.4±1.2%; P>0.1 [male] and control littermates 29.5±1.9% versus Adamts7+/tm1b mice 24.4±1.0%; P<0.05 [female]; FIG. 14B and FIG. 14C). Next we analyzed the plaque content from aortic root sections on the ApoE knockout background. Similar to our results on the AAV-PCSK9 model, we did not observe a significant difference in aortic root for any of the genotypes on the ApoE KO background (FIG. 15A-FIG. 15F).

In a parallel cross, we examined WT, heterozygous Adamts7+/E373Q and homozygous Adamts7 E373Q/E373Q mice after administration of a high-fat Western diet for 10 weeks. From this cross, we observed a significant decrease in lesion formation in the aortic arch region between WT and Adamts7 E373Q/E373Q mice both in male and female mice (control littermates 24.5±1.8% versus Adamts7 E373Q/E373Q mice 18.0±1.2%; P<0.01 [male] and control littermates 26.0±2.0% versus Adamts7 E373Q/E373Q mice 19.3±1.2%; P<0.05 [female], FIG. 14D-FIG. 14F). We also observed an intermediate phenotype in Adamts7+/E373Q heterozygous animals approaching significance for both sexes (control littermates 24.5±1.8% versus Adamts7+/E373Q mice 20.0±1.2%; P=0.09 [male] and control littermates 26.0±2.0% versus Adamts7+/E373Q mice 22.8±1.1%; P>0.1 [female], FIG. 14E and FIG. 14F). Collectively our results on the ApoE KO atherogenic background corroborate our data from the AAV-PCSK9 atherogenic model and support a dosage-dependent protective effects from ADAMTS7 catalytic inhibition.

Adamts7 Catalytic Function is Responsible for VSMC Migration

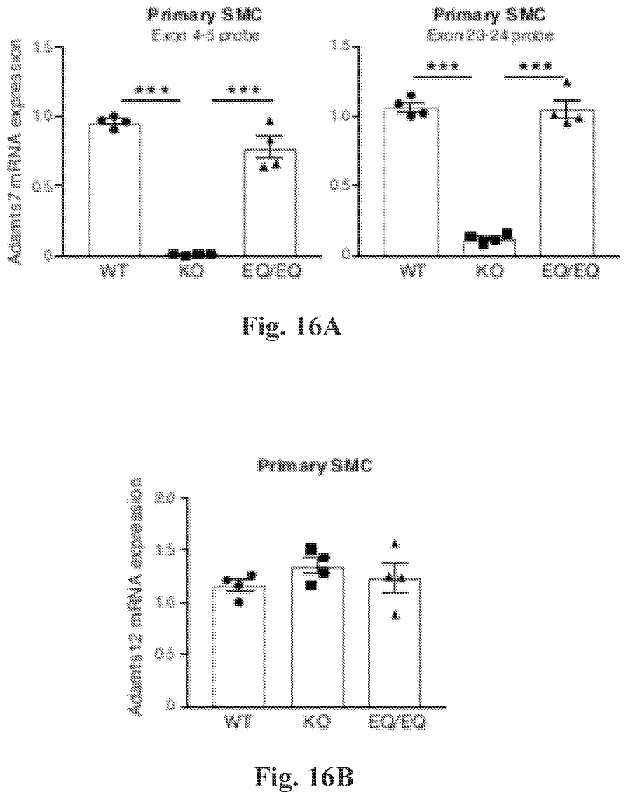

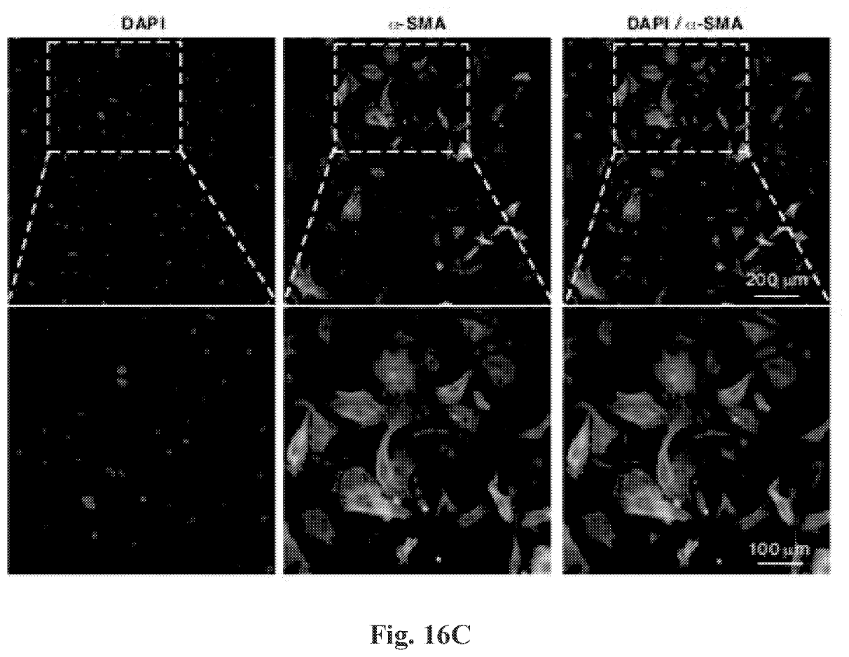

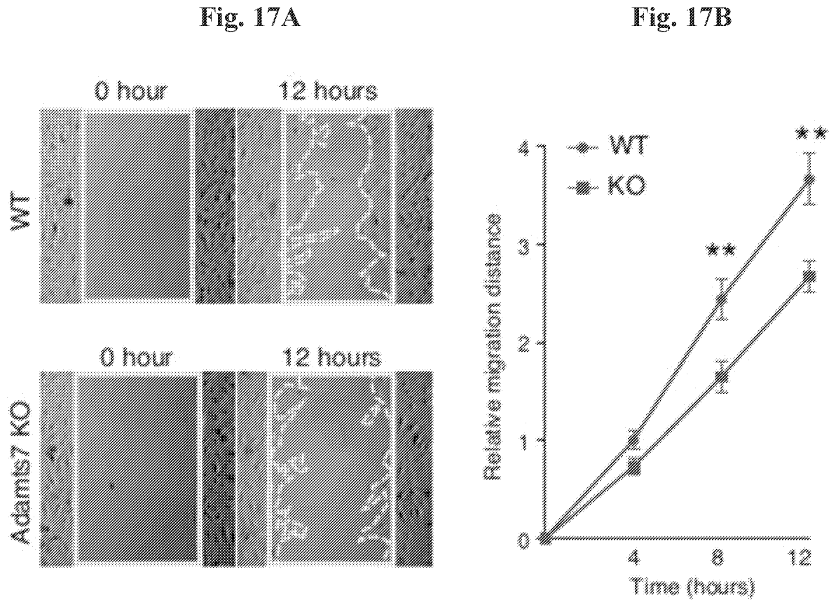

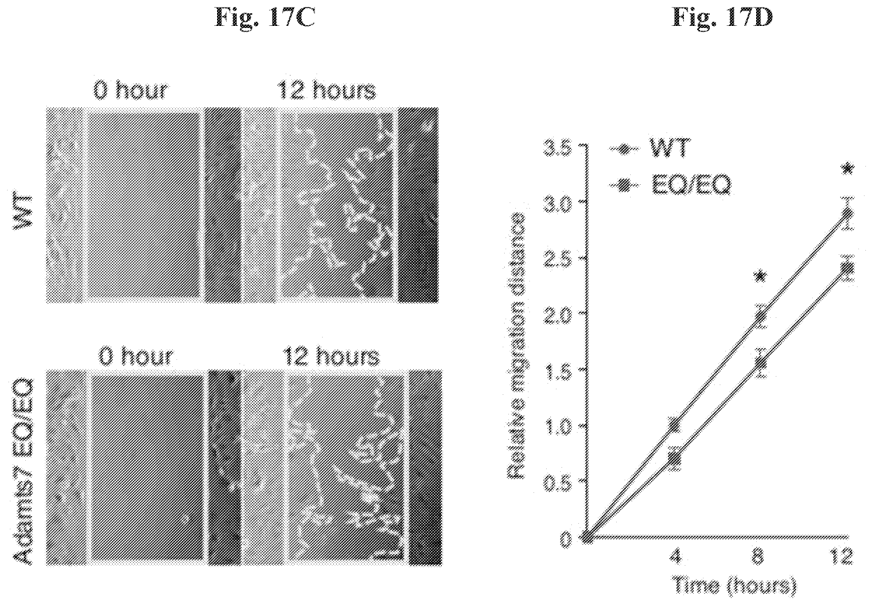

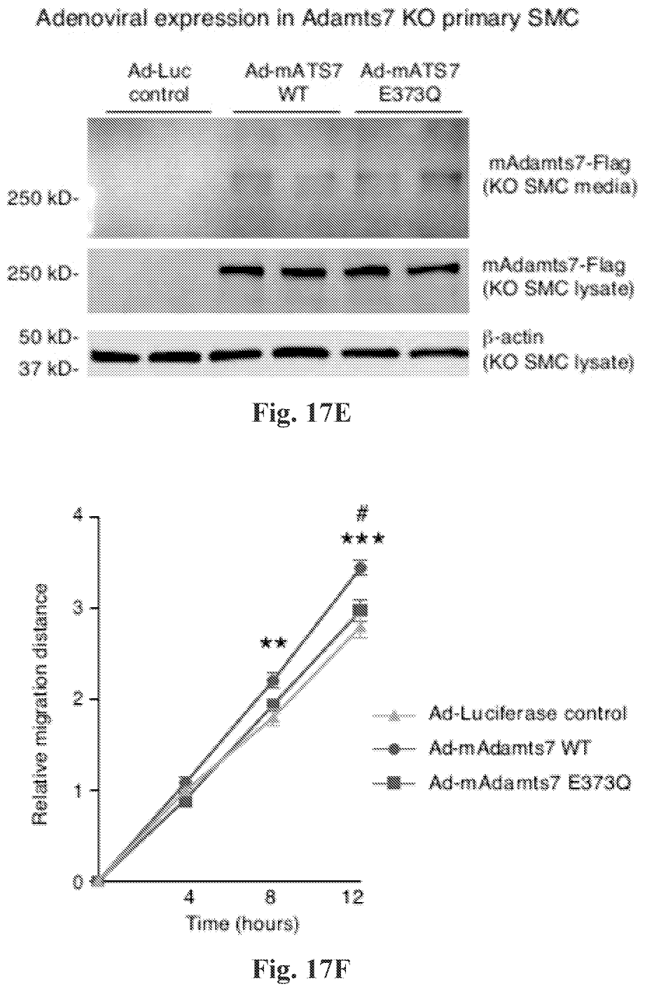

ADAMTS7 has been implicated in the regulation of VSMC migration. To reveal the effect of ADAMTS7 catalytic inactivation on VSMC migration, we performed wound healing assays on the primary VSMCs harvested from WT, Adamts7 KO and E373Q/E373Q mice. We verified that ADAMTS7 catalytic inactivation and Adamts7 gene knockout did not affect Adamts7 or Adamt12 mRNA expression in primary VSMCs (FIG. 16A and FIG. 16B). Our results from the wound healing assay validated reduced migration of Adamts7 KO VSMCs compared with WT VSMCs consistent with the previous literature (Bauer et al. (2015) Circulation 131: 1202-1213) (FIG. 17A and FIG. 17B). Subsequently VSMCs from E373Q/E373Q mice were tested for migration and showed less migratory activity compared to littermate WT VSMCs (FIG. 17C and FIG. 17D), supporting a role for ADAMTS7 catalytic function in promoting VSMC migration.

To test whether catalytic inactivation of ADAMTS7 counteracts pro-migratory effects of ADAMTS7, VSMCs from Adamts7 KO mice were infected with Ad-Luciferase, Ad-mouse Adamts7 WT (mAts7 WT) or Ad-mouse Adamts7-E373Q (mE373Q) and were subject to wound healing assays. As expected, overexpression of ADAMTS7-WT, but not E373Q, promoted VSMC migration (FIG. 17E and FIG. 17F). Western blot analyses confirmed no differences in protein levels in cultured media and cell lysates between ADAMTS7-WT and ADAMTS7-E373Q, indicating that pro-migratory effects were abolished by the E373Q substitution without affecting the protein levels (FIG. 17E). In summary our in vitro studies indicate that ADAMTS7 catalytic activity promotes VSMC migration.

ADAMTS7 Coding Variant Associated with CAD Risk Affects Secretion and VSMC Migration

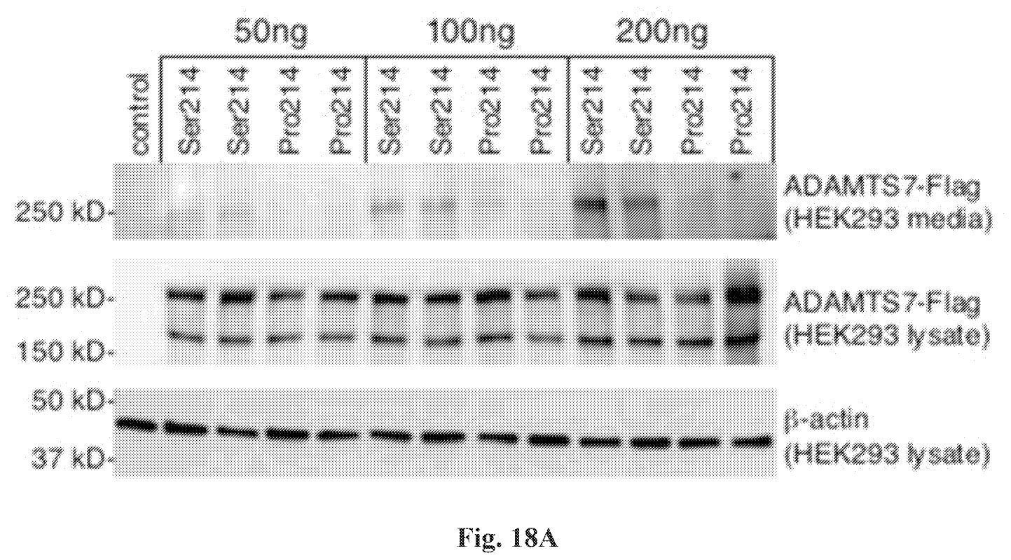

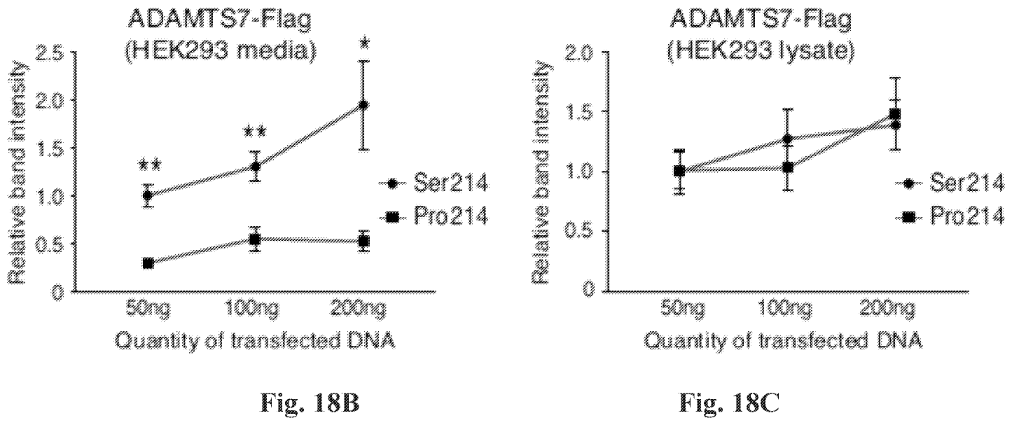

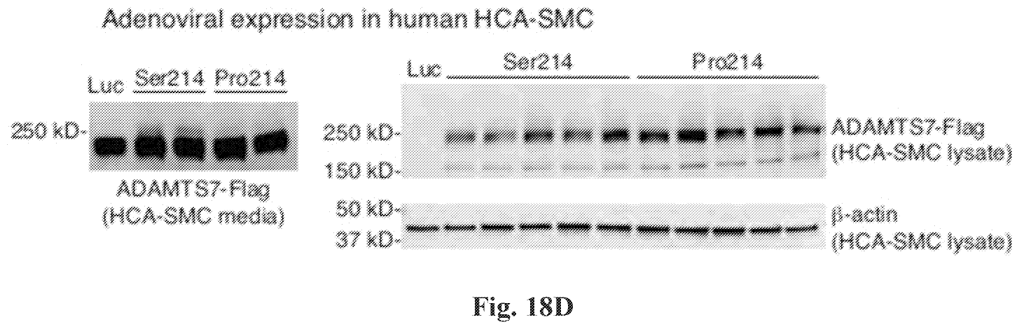

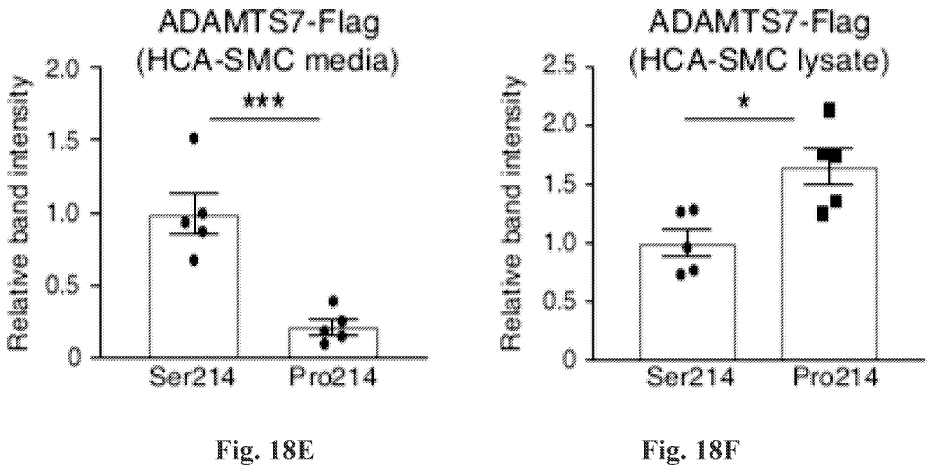

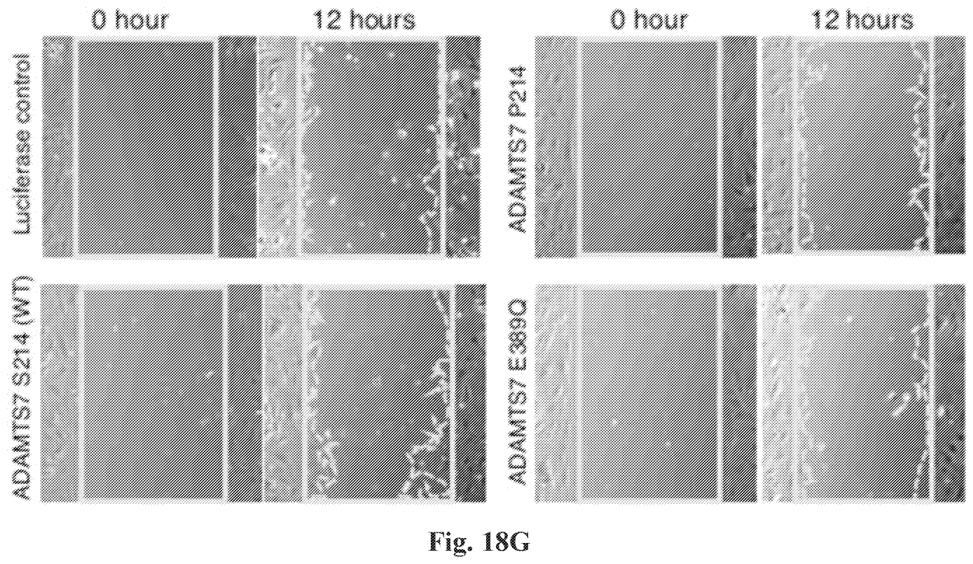

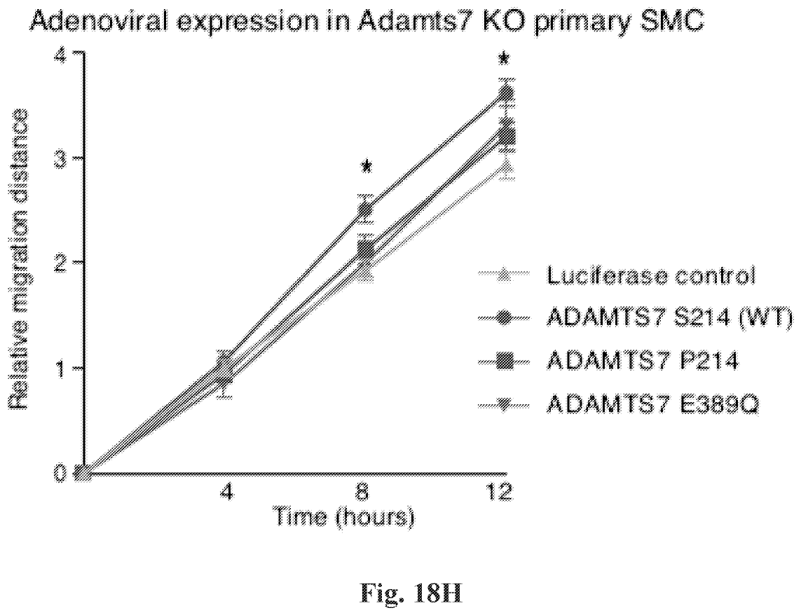

Having established that inhibition of Adamts7 catalytic activity prevents atherosclerosis and VSMC migration, we then sought to determine the impact of a coding variant in ADAMTS7 that is associated with CAD risk. A nonsynonymous variant in ADAMTS7, rs3825807, results in serine to proline substitution at amino acid 214. To further examine the importance of this coding variant, we generated full-length human expression vectors (1-1686 a.a. with a c-terminal 3×Flag tag) carrying either the common risk allele Ser214 or the protective allele Pro214. Transfection into HEK293T cells show that Ser214 is secreted into the media in a dose dependent manner while Pro214 is hindered at all doses tested (FIG. 18A and FIG. 18B). Analysis of the lysate shows similar levels for Ser214 and Pro214 expressed proteins at both the highly glycosylated 270 kDa mature and immature 180 kDa forms (FIG. 18C). Next, we transferred the full-length ADAMTS7 alleles into an adenovirus expression system and tested the secretion phenotype in human coronary artery smooth muscle cells (HCA-SMC). Once again, we observed a reduction in Pro214 secretion into the media compared to the Ser214 allele (FIG. 18D and FIG. 18E). In contrast, we detected an increase level of mature Pro214 ADAMTS7 in the HCASMC lysates (FIG. 18F).

Finally, to test the effects of the human risk and protective alleles, we harvested primary VSMC from the Adamts7 KO mice and analyzed the lateral migration induced by adenoviral expression of human ADAMTS7 (FIG. 18G). Expression of the human Ser214 risk allele resulted in the highest amount of VSMC migration which was significant compared to a Luciferase control (FIG. 1811). There was a reduction in migration for the Pro214 allele, consistent with the observed reduction of ADAMTS7 levels detected in the media. Because extracellular matrix degradation has been implicated in regulation of VSMC migration, we hypothesized that ADAMTS7 catalytic function was responsible for ADAMTS7-mediated VSMC migration. To eliminate catalytic function for human ADAMTS7, we altered the HExxH motif with a glutamate to glutamine substation similar to the catalytic mutant mouse allele (FIG. 9A). The ADAMTS7 Glu389Gln (EQ) catalytic mutation was made in the Ser214 allele construct and transferred to an adenovirus expression system. Expression and secretion of the EQ catalytic mutant was comparable to Ser214 allele (data not shown). However, the amount of induced migration from the ADAMTS7 EQ catalytic mutant was reduced compared to the Ser214 allele, and was equivalent to the hypomorphic Pro214 allele (FIG. 18H). Our results suggest that loss of catalytic activity may be functionally equivalent to lower levels of ADAMTS7 expression in VSMC.

### Discussion

Population based genetic studies have identified more than a hundred disease associated genetic loci for CAD. However, it remains challenging to leverage these associations into causative genes, biological mechanisms and ultimately into new therapies (Erdmann et al. (2018) Cardiovasc Res 114: 1241-1257). In most cases GWAS provides a location with candidate gene(s) but does not intrinsically define directionality or mechanism for disease. This understanding is essential for translating a GWAS hit into a druggable target and requires validation in cell-based assays and model organisms. From the integrative analysis of multiple variants, there is support for ADAMTS7 as the causal gene at the human chromosome 15q25 locus (Reilly et al. (2011) Lancet 377: 383-392; Schunkert et al. (2011) Nat Genet 43: 333-338; C4D Genetics Consortium (2011) Nat Genet 43: 339-344). ADAMTS7 loss of function correlates with atheroprotection in the mouse and provides support for a therapeutic antagonist approach (Bauer et al. (2015) Circulation 131: 1202-1213). As a secreted enzyme in a gene family with several examples of small molecule (Muller et al. (2016) Biochem Biophys Res Commun 471: 380-385) and antibody-based inhibitors (Santamaria and de Groot (2019) Br J Pharmacol 176: 52-66), ADAMTS7 is a potential therapeutic target, yet a mechanism for disease independent of catalytic function remained a distinct possibility.

Here, we have demonstrated for the first time that protease activity is essential for proatherogenic effects of Adamts7. We created a catalytically inactivated Adamts7 mouse line and compared this to an Adamts7 knockout mouse in two different atherogenic backgrounds. In each case, we observed a critical role for both Adamts7 catalytic activity and dosage in atherosclerosis. In aggregate, our in vivo studies provide compelling support for ADAMTS7 catalytic inhibition as a therapeutic approach for atherosclerosis.

In both of our atherogenic mouse models, we observed that a reduction in atherosclerosis development was most prominent in the aortic arch and brachiocephalic region. We did observe a reduction in atherosclerosis in the lower sections of the aorta but the timeframes of atherogenesis we choose for our study did not result in a robust accumulation in plaques for these regions in our animal facility. Therefore, a limitation in our study is that most of the beneficial effects were based from measurements in the thoracic aortic region. Although we did not detect a significant difference in the amount of plaque in the aortic root, we did observe a reduction in α-smooth muscle actin (SMA) staining at this location consistent with a decrease in SMC content in both Adamts7 KO and catalytic mutant homozygotes. Careful investigation of ADAMTS7 expression by X-gal staining in Adamts7+/tm1b heterozygotes revealed a transient window of expression in developing and early plaques in the aortic root. Remarkably this expression did not persist in later stages of plaque development. Our observations are consistent with a transient ADAMTS7 expression response given an atherogenic or vascular injury challenge (Bauer et al. (2015) Circulation 131: 1202-1213; Wang et al. (2009) Circ Res 104: 688-698). Coupled with our in vitro results showing a reduction in SMC migration in both loss of function and catalytic mutants, and rescue of migration with only the active form of ADAMTS7, our data supports a model in which ADAMTS7 facilitates atherosclerosis through VSMC phenotypic switching mediated through catalytic function.

We have also examined the biological effect of a CAD associated coding variant, rs3825807 (Ser214Pro). The hypomorphic secretion of the full-length Pro214 protective allele is similar to those published with a truncated ADAMTS7 (1-460 a.a.) containing only the prodomain and catalytic domain (Pu et al. (2013) Am J Hum Genet 92: 366-374). We found that the Pro214 allele resulted in hypomorphic function due to both decreased Adamts7 secretion and reduced vascular smooth muscle cell migration. Expression of the full-length Ser214 risk variant stimulated VSMC migration compared to control, Pro214 or EQ catalytic mutant variants in cells devoid of ADAMTS7 function. Thus, our results are in keeping with prior reports that this variant is likely to explain at least a portion of the CAD associated risk at this locus (Pereira et al. (2016) Physiol Genomics 48: 810-815; Chan et al. (2017) J Am Heart Assoc 6(11): e006928; Bengtsson et al. (2017) Sci Rep 7: 3753; Li et al. (2019) Medicine (Baltimore) 98: e17438; Pu et al. (2013) Am J Hum Genet 92: 366-374).

Before ADAMTS7 was reported as CAD GWAS locus, it was demonstrated that ADAMTS7 knockdown prevented, while ADAMTS7 overexpression increased, neointima formation in a rat carotid artery vascular injury model (Wang et al. (2009) Circ Res 104: 688-698). Mouse knockout studies confirmed that total loss of function reduced neointima formation in the carotid (Kessler et al. (2015) Circulation 131: 1191-1201) and femoral arteries (Bauer et al. (2015) Circulation 131: 1202-1213). We have yet to examine our catalytic mutant mice in a vascular injury model to determine if this phenotype is mediated purely through catalytic function or if the additional domains of ADAMTS7 contribute to non-catalytic protein-protein based interactions in the extracellular matrix. For example, it was shown that catalytic mutations in the paralog ADAMTS12 were able to partial rescue the Adamts12 loss of function effects in an aortic ring endothelial cell sprouting assay (El Hour et al. (2010) Oncogene 29: 3025-3032). Additionally, there has been a lack of characterized ADAMTS7 substrate cleavage sites for the reported substrates COMP and TSP1 that may contribute to the observed vascular phenotype. Thus, a limitation to our study would be direct link to an ADAMTS7 substrate with biological effects that is largely unchanged in both our loss of function and catalytic mutant homozygotes. Recently an unbiased ADAMTS7 substrate cleavage site identification method using terminal amine isotopic labeling of substrates (TAILS) was employed to search for candidate sites from a human fibroblast secretome (Colige et al. (2019) J Biol Chem 294: 8037-8045). Further work will be needed to link these candidate cleavage sites, or others identified by similar TAILS experiments, to hone in an endogenous substrate or substrates responsible for the vascular phenotypes observed in ADAMTS7 catalytic mutant mice.

In conclusion, our current study provides compelling evidence that Adamts7 dosage and catalytic activity can be modulated for therapeutic benefit. These findings suggest that ADAMTS7 catalytic activity would be a promising therapeutic target for CAD.

### Methods

Plasmid DNA and Adenoviral Expression Vectors

Full-length human ADAMTS7 and full-length mouse Adamts7 were Gibson cloned into the pcDNA3.4 expression vector with carboxyl terminal 3×FLAG tag (GGGS linker preceding DYKDHDGDYKDHDIDYKDDDDK tag). Human ADAMTS7 S214P and E389Q, and Mouse ADAMTS7 E373Q substitutions were generated using QuikChange Mutagenesis (Agilent). Human ADAMTS7 and mouse Adamts7 inserts were sub-cloned into a CMV driven Adenoviral vector (Ad5 with E1/E3 deletion) to generate custom adenoviruses from Vector Biolabs. Adeno-CMV-Luciferase (Ad-Luc) from Vector Biolabs was used as a negative control.

Mammalian Cell Culture

HEK293 cells were cultured in DMEM (11965092, Life Technologies, Inc) containing 10% fetal bovine serum (FBS; 16140071, Life Technologies, Inc) and 1% penicillin-streptomycin (15140163, Life Technologies, Inc) at 37° C. in a 5% CO₂ humidified chamber. The cells were transiently transfected with empty vector, ADAMTS7-Ser214 or ADAMTS7-Pro214 expression plasmids using FuGENE 6 (E2692, Promega). Forty-eight hours after transfection, conditioned media were collected and the cells were lysed in cell lysis buffer (50 mmol/L Tris pH 7.5, 0.1% sodium dodecyl sulfate, 0.5% NP-40).

Human coronary artery smooth muscle cells (HCASMCs) were cultured on collagen coated 12 well plate (354500, CORNING) in SMC medium (LL-0014, Lifeline) containing 5% FBS. For preparation of cell lysates for western blot analyses, the cells were incubated with 20 MOI of Ad-Luciferase, Ad-ADAMTS7-Ser214 or Ad-ADAMTS7-Pro214 in Opti-MEM (51985034, Life Technologies, Inc) supplemented with 5 μg/ml polybrene (TR-1003-G, SIGMA) for 4 hours and grown in SMC medium containing 5% FBS for 48 hours before lysed in the cell lysis buffer. For preparation of conditioned media for western blot analyses, the cells were incubated with 100 MOI of Adenoviral vectors in OPTI-MEM medium supplemented with 5 μg/ml polybrene. Four hours after the infection, the cells were maintained in SMC medium containing 5% FBS for 24 hours. Subsequently the cells were incubated in SMC medium without FBS for additional 24 hours before the conditioned medium collection. Cells at passage 4 to 6 were used for experiments.

Mouse VSMCs were isolated from the aorta of WT, KO and mutant mice aged 6 to 8 weeks as described previously according to the “explant method” (Metz et al. (2012) Methods Mol Biol 843: 169-176), with minor modifications. Briefly, aorta was flushed in place, then dissected out, opened longitudinally, transferred to a sterile environment and rinsed again in rinsed in sterile PBS. Tissue was placed with the intima facing down on a collagen I coated petri dish, and a sterile glass slide was placed on top to maintain the tissue in contact with the dish and favor VSMC outward migration. After 7 days of culture in RPMI1640 (61870127, Life Technologies, Inc) containing 20% FBS and 1% penicillin-streptomycin (15140163, Life Technologies, Inc) cells were detached by Accutase (#07922, STEMCELL TECHNOLOGIES) and expanded one passage before the experiment. For preparation of cell lysates and conditioned media for western blot analyses, the cells were incubated with 50 MOI of Ad-Luciferase, Ad-ADAMTS7-Ser214, Ad-ADAMTS7-Pro214, Ad-mAdamts7 WT or Ad-mAdamts7-E373Q in Opti-MEM supplemented with 5 μg/ml polybrene for 4 hours and were allowed to grow in RPMI1640 containing 20% FBS for 24 hours. Subsequently the cells were incubated in RPMI1640 containing 0.5% FBS for additional 24 hours before the conditioned medium collection. The VSMC purity was verified by anti-SMA antibody staining and cells at passages 2 were used in all experiments (FIG. 16A-FIG. 16C).

Western Blot Analysis

Liver tissues, cell lysates and conditioned media were subject to western blot analyses. Protein was separated by sodium dodecyl sulfate-polyacrylamide gel electrophoresis (SDS-PAGE) using 4% to 20% gradient gels (456-1095, Bio-RAD) or 7.5% gels (456-1025, Bio-RAD) and transferred to nitrocellulose membranes (1620215, Bio-RAD). Membranes were blocked with 3% BSA for 1 hour at room temperature. Membranes were incubated in TBST with 0.5% BSA at 4° C. overnight with peroxidase (HRP) conjugated anti-FLAG M2 antibody (A8592, Sigma), rabbit anti-mouse LDL receptor antibody (3839, BioVision) or HRP-conjugated goat anti-actin antibody (sc-1616, Santa Cruz Biotechnology). After incubated with anti-mouse LDL receptor antibody, the membranes were incubated with HRP-conjugated goat anti-rabbit antibody (A16096, Invitrogen) at room temperature for 1 hour. Images were captured using Amersham Imager 600 (Amersham).

Vascular Smooth Muscle Migration Assay

A culture-insert 3 well (80369, ibidi) was cut in half parallel to the short axis and prepared on collagen-coated 12 well cell culture plate (354408, Corning). For protein overexpression studies, 940 ul and 60 ul of a 1.2×10⁵ cells/ml aliquot (primary Adamts7 KO VSMCs) were placed outside and inside the insert, respectively. The cells were allowed to adhere for 5 hours and then infected with 50 MOI of Ad-Luciferase, Ad-hADAMTS7-Ser214, Ad-hADAMTS7-Pro214, Ad-mAdamts7 WT and Ad-mAdamts7-E373Q in Opti-MEM supplemented with 5 μg/ml polybrene. Four hours after the infection, the cells were maintained in RPMI1640 containing 20% FBS for 16 hours and thereafter cultured in RPMI1640 containing 0.5% FBS for 24 hours prior to wound healing assay. To test the effect of Adamts7 genotype on VSMCs migration, the same number of primary WT VSMCs, Adamts7 KO VSMCs and E373Q/E373Q VSMCs were placed on the culture plate and maintained in RPMI1640 containing 0.5% FBS for 24 hours. Cell-free gap was created by removing the insert and fresh medium containing 0.5% FBS was added after PBS wash. Three fields were imaged in each well to record migration distance at 0, 4, 8 and 12 hours. The distance that cells migrated from the initial wound edge (initial wound area−remaining wound area) was measured using Image J.

Generation of Mouse Adamts7 Alleles

Publicly available Adamts7 tm1a (KOMP) Wtsi “knock-out first/conditional ready” mice were obtained from UC Davis and resuscitated on the C57BL/6J strain background. The Adamts7 tm1a allele contains a gene-trap LacZ reporter and PGK-Neo inserted into Adamts7 intron 4, followed by loxP flanked Adamts7 exons 5 and 6 encoding the catalytic domain. Adamts7 tm1a mice were bred with EIIa-Cre mice (JAX 003724) to remove the PGK-Neo and loxP flanked exons, generating the tm1b “LacZ tagged null allele”. WT, tm1a and tm1b alleles were distinguished using a 3 primer PCR assay (3′loxP F: TAGAATAGCGGGCTCTCGTG (SEQ ID NO: 34), LacZ F: TTTCCATATGGGGATTGGTG (SEQ ID NO: 35), 3′loxP R: CTCGGGAATGGAATCTTGAC (SEQ ID NO: 36)) generating PCR products WT=441, tm1a=352, tm1b=580.

The Adamts7 E373Q catalytic mutant allele was generated using CRISPR HDR at the Harvard Genome Modification Facility on the C57BL/6J strain background. Three candidate guides in Adamts7 exon 7 were chosen based on proximity to the c.1117G->C (p.Glu373G1n) mutation site and computational prediction scores for low off target activity. Targeting efficiency was analyzed following transfection and Puro selection of guides cloned into the px459 vector in mouse NIH3T3 cells. From this evaluation, guide sequence TTACCTGTGTCCTAGCTCGT (SEQ ID NO: 37) was chosen and the corresponding crRNA was synthesized by IDT. Custom crRNA was complexed with CRISPR-Cas9 tracrRNA and IDT S.p. HiFi Cas9 Nuclease 3NLS. An asymmetric 132 base ssODN was designed to serve as a template for HDR with an additional silent mutation at c.1113C->T (p.Ala371Ala) to disrupt the PAM site:

TGGGCCTGTCTCATGTGTCTGGCTTGTGCCACCCTCAGCTCAGCTGTAGCGTCA GTGAGGATACTGGCATGCCACTGGCCTTCACTGTGGCTCACCAGCTAGGACAC AGGTAACTCATCTTCCCTCACCTCT (SEQ ID NO: 38) (mismatches are underlined). CRISPR founders were genotyped by sequencing Adamts7 exon 7 for the c.1113C->T,c.1117G->C mutations and the E373Q allele was backcrossed to C57BL/6J mice. WT and E373Q alleles were distinguished using a 3 primer PCR assay (Adamts7 in06F: CCTTCTGAGGTCCCAGTGAG (SEQ ID NO: 39), Adamts7 ex07Rp: ctgcaggaattcgatatcaagcttatcgataCCTGTGTCCTAGCTCGTGG (SEQ ID NO: 40), Mut Adamts7 E373Q R: CCTGTGTCCTAGCTGGTGA (SEQ ID NO: 41)) generating PCR products WT=377, E373Q=346. The 3′ regions of the WT and E373Q reverse primers are specific to the designed mutations and the WT reverse primer contains an additional 31 bases to distinguish the products.

Guide sequence TTACCTGTGTCCTAGCTCGT (SEQ ID NO: 37) is unique to Adamts7; the next closest matches in the genome contain three or more mismatches. From a list of 50 off-target sites, four were nearby coding regions for Noc4l (CACCCTGTGTCCTAGCTCCT) (SEQ ID NO: 42), Zfp446 (GAACCTGTGTCCCAGCTCTT) (SEQ ID NO: 43), Ncoa2 (TCACCTGGGTCCGAGCTGGT) (SEQ ID NO: 44) and Drosha (CTACCTGTTTCCTAGCTTGG) (SEQ ID NO: 45). Heterozygous+/E373Q offspring were used to assess off target CRISPR activity near these coding regions using genomic PCR and Sanger sequencing. No mutations were found to suggest off-target events. To assess equal transcription of the WT and E373Q alleles, cDNA from heterozygous +/E373Q and WT hearts were amplified using RT-PCR from exons 5-9 and sequenced using forward and reverse primers. All animal experiments were reviewed and approved by the Institutional Animal Care and Use Committees of the Broad Institute of MIT and Harvard.

Allelic Expression Imbalance Analysis

To assess equal transcription of the WT and E373Q alleles, cDNA from heterozygous+/E373Q and WT hearts were amplified using RT-PCR from exons 5-9 (Adamts7 ex05 F: CGATCATGAACATGGTGGCTGG (SEQ ID NO: 46), Adamts7 ex08 R: TGGACGGTCATCTAAGCACAGG (SEQ ID NO: 47)) and genomic PCR was amplified from tail snip DNA using primers flanking exon 7 (Adamts7 in06 F: CCTTCTGAGGTCCCAGTGAG (SEQ ID NO: 39), Adamts7 in07 R: ACTCCCTCAGGTTTCGTGAC (SEQ ID NO: 48)). The RT-PCR and genomic PCR products from heterozygous+/E373Q and WT mice were sequenced using forward and reverse primers. Chromatograms were compared at the synonymous PAM site (GCC to GCT) and E373Q (GAG to CAG) substitutions. Heterozygotes contained two peaks at both of these positions. The relative peak sizes for the WT alleles was diminished in heterozygotes compared to the control WT sequence reads with two WT alleles. Peak sizes from +/EQ heterozygous genomic PCR (with a 1:1 ratio of WT and EQ alleles) was equivalent to the sequence RT-PCR products consistent with a similar 1:1 ratio of expressed Adamts7 alleles. Representative forward reads are displayed in FIG. 9B.

Real-Time RT-PCR Expression Analysis

Aortas and hearts were dissected from WT, Adamts7 KO and E373Q/E373Q mice at 10 weeks of age after perfusion with PBS. Following tissue homogenization in TRIzol reagent (Life Technologies) using a BeadBug™ prefilled tube and Bead Blaster 24 total RNA was extracted. RNeasy Mini Kit (74104, QIAGEN) was used to extract total RNA from mouse primary VSMCs according to manufacturer's protocol. For RT, iScript Reverse Transcription Supermix for RT-qPCR (1708841, Bio-RAD) was used. Quantitative PCR was performed using a SsoAdvanced Universal Probes Supermix (172-5284, Bio-RAD) and C1000 Touch Thermal Cycler (Bio-RAD) according to the manufacture's protocol. The following Taqman probes were used to amplify mAdamts7, mAdamts12 and TATA-binding protein (Tbp): mAdamts7 Mm01239069 and Mm01239067, mAdamts12 Mm01298775, Tbp Mm00446973. Amplification reactions were performed in triplicate and gene expression levels were analyzed by delta-delta CT method. Tbp was used as an endogenous control reference.

Atherogenic Mouse Models

In the first model, 10-week-old mice were injected intravenously thorough the retro-orbital sinus plexus with 2.0×10¹¹ genome copy (GC) of AAV8 expressing a gain-of-function mutant of mouse PCSK9 (rAAV8-D377Y-mPCSK9) and subsequently fed Western diet (D12079Bi, Research Diets, Inc) as previously described (Bjorklund et al. (2014) Circ Res 114: 1684-1689). Whole blood samples (non-fasting) were collected via retro-orbital sinus plexus for plasma PCSK9 and total cholesterol before vector injection and at 1, 2, 3, 4, 6, 8, 12 and 16 weeks after injection. For analysis of plasma PCSK9 levels, a murine PCSK9-specific ELISA was used (MPC900, R&D Systems). Plasma total cholesterol was measured with a cholesterol fluorometric assay kit (10007640, Cayman Chemical). Sixteen weeks after injection, mice were deeply anesthetized by a inhalation of Isoflurane and was then transcardially perfused with 10 ml of PBS. For analysis of hepatic LDL receptor levels, liver tissue was dissected and homogenized in tissue lysis buffer (50 mmol/L Tris pH 7.5, 0.1% sodium dodecyl sulfate, 1.0% NP-40) using a BeadBug™ prefilled tube (Z763799, Sigma-Aldrich) and Bead Blaster 24 (Benchmark D2400). After perfusion with 10 ml of 4% paraformaldehyde, the aorta was dissected from the middle of the left ventricle to the bifurcation of the hepatic artery. Dissected aortas were post-fixed overnight in 4% paraformaldehyde at 4° C. For aortic root lesion analysis, the samples were cut in the ascending aorta, and the proximal samples containing the aortic sinus were embedded in OCT compounds (4583, Sakura Finetek). In the second model, Adamts7 KO mice and E373Q/E373Q were backcrossed with atherogenic ApoE KO background. Ten-week-old mice were fed Western diet and plasma total cholesterol levels and atherosclerotic lesion formation in the aortic arch and aortic root were analyzed at 10 weeks of the Western diet using the same method as the first model.

Atherosclerotic Lesion Assessment

Five consecutive sections (10 μm thickness), spanning 550 μm of the aortic sinus, were obtained from each mouse using cryostat and stained with Oil Red 0 (00625, Sigma). Stained sections were digitally captured. For quantitative analysis of atherosclerosis, the total lesion area of 5 separate section from each mouse was obtained with the use of the Image J (National Institute of Health) as previously described (Sasaki et al. (2009) Circulation 120: 1996-2005). For en face lesion analysis, the aorta was excised from the proximal ascending aorta to the common iliac artery bifurcation and fixed in 4% paraformaldehyde (PFA) (BM-155, Boston BioProducts). After the adventitial tissue was carefully removed the aorta was opened longitudinally, stained with Oil Red 0, pinned on a black wax surface, and captured digitally with a digital camera. The percentage of stained lesion area per total area of the aortic arch was determined by Image J.

Immunohistochemical Analysis of Atherosclerotic Lesions

Immunohistochemistry was performed on 10 μm tissue sections of mouse aortic roots using antibodies to identify macrophages (Alexa Fluor 647 anti-mouse CD68; 37004, Biolegend) and smooth muscle cells (Cy3 anti-mouse-a-Smooth Muscle; C6198, SIGMA). Cryosections were permeabilized in 0.05% Tween 20 for 20 min and blocked in TBST containing 5% bovine serum albumin (BSA; AP-4510-80, SeraCare) for 30 min at room temperature. The sections were incubated overnight with the antibodies at 4° C., washed twice with TBST and incubated with Hoechst 33342 (3570, Invitrogen) for 1 hours. Sections were washed twice again, and slides were mounted with Fluorescence Mounting Medium (S3023, DAKO). Staining with Masson's trichrome was performed using Masson's trichrome stain Kit (25088, Polysciences Inc) to delineate the fibrous area. Images are acquired using a fluorescence microscope and the percentage of the stained area (the stained area per total atherosclerotic lesion area) was calculated.

β-Gal Staining of Atherosclerotic Lesions

For β-gal staining in aortic roots, aortic roots were dissected from Adamts7 heterozygous KO mice at different weeks following the AAV-PCSK9 injection (6, 8, 12 and 16 weeks) after perfusion with 10 ml PBS. The samples were embedded in OCT compound. X-gal staining was performed on 10 μm cryosections using β-gal Staining Set (#11828673001, Roche) according to manufacturer's instruction. Briefly, after an hour fixation in 2% paraformaldehyde, tissue sections were incubated with β-gal staining solution at 37° C. for 48 hours. Quantification of β-gal positive cells was done by counting positively stained cells in atherosclerotic lesions.

Statistical Analysis

Two-tailed Student t test or one-way ANOVA with Tukey's test was used to detect significant differences between 2 groups. A value of P<0.05 was considered statistically significant. For all data, error bars indicate s.e.m. For statistical analysis, Graphpad Prism 8 (GraphPad Software) was used.

What is claimed is:

1. A recombinant nucleic acid for expression of an ADAMTS-7 polypeptide that comprises a functional segment of a rodent prodomain of ADAMTS-7 as a first portion and a functional segment of human catalytic domain of ADAMTS-7 as a second portion.

2. The recombinant nucleic acid of claim 1 comprising a functional segment of human CD domain of ADAMTS-7 as the second portion.

3. A recombinant nucleic acid for expression of an ADAMTS-7 polypeptide, wherein the polypeptide comprises
a first portion having a sequence identity of >80% with the sequence of residues 1-217 of SEQ ID NO: 1 or with the sequence of residues 1-217 of SEQ ID NO: 2; and
a second portion having a sequence identity of >80% with the sequence of residues 218-518 of SEQ ID NO: 1.

4. The recombinant nucleic acid of any one of claims 1 to 3, wherein the first portion of the polypeptide comprises residues 1-217 of SEQ ID NO: 1 or residues 1-217 of SEQ ID NO: 2, and/or wherein the second portion amino acid sequence comprises residues 218-518 of SEQ ID NO: 1.

5. A recombinant nucleic acid for expression of an ADAMTS-7 polypeptide, wherein the recombinant nucleic acid encodes for a recombinant polypeptide that comprises
a first portion having an amino acid sequence that aligns with a functional segment of an ADAMTS-7 prodomain amino acid sequence from a first species with a Needleman-Wunsch score greater than 700 when BLOSUM62 matrix, a gap opening penalty of 11, and a gap extension penalty of 1 are used; and
a second portion having an amino acid sequence that aligns with a functional segment of an ADAMTS-7 CD domain amino acid sequence from a second species with a Needleman-Wunsch score greater than 1000 when BLOSUM62 matrix, a gap opening penalty of 11, and a gap extension penalty of 1 are used,
wherein optionally the first species is rat and the second species is human.

6. A recombinant nucleic acid for expression of an ADAMTS-7 polypeptide, wherein the recombinant nucleic acid encodes for a recombinant polypeptide that comprises
a first portion having an amino acid sequence that aligns with a functional segment of an ADAMTS-7 prodomain amino acid sequence from a first species with a Needleman-Wunsch score greater than 700 when BLOSUM62 matrix, a gap opening penalty of 11, and a gap extension penalty of 1 are used; and
a second portion having an amino acid sequence that aligns with a functional segment of an ADAMTS-7 catalytic domain amino acid sequence from a second species with a Needleman-Wunsch score greater than 700 when BLOSUM62 matrix, a gap opening penalty of 11, and a gap extension penalty of 1 are used,
wherein optionally the first species is rat, and the second species is human.

7. The recombinant nucleic acid of any one of claims 1 to 6, wherein the motif RQQR within the first portion of the polypeptide is altered, preferably into RQKR.

8. A recombinant nucleic acid for expression of an ADAMTS-12 polypeptide that comprises a functional segment of a rodent prodomain of ADAMTS-12 as a first portion and a functional segment of human catalytic domain of ADAMTS-12 as a second portion.

9. The recombinant nucleic acid of claim 8 comprising a functional segment of human CD domain of ADAMTS-12 as the second portion.

10. A recombinant nucleic acid for expression of an ADAMTS-12 polypeptide that comprises
a first portion having a sequence identity of >80% with the sequence of residues 1-244 of SEQ ID NO: 15, and
a second portion having a sequence identity of >80% with the sequence of residues 245-547 of SEQ ID NO: 15.

11. The recombinant nucleic acid of any one of claims 8 to 10, wherein the first portion comprises residues 1-244 of SEQ ID NO: 15, and/or wherein the second portion amino acid sequence comprises residues 245-547 of SEQ ID NO: 15.

12. A recombinant nucleic acid for expression of an ADAMTS-12 polypeptide, wherein the recombinant nucleic acid encodes for a recombinant polypeptide that comprises
a first portion having an amino acid sequence that aligns with a functional segment of an ADAMTS-12 prodomain amino acid sequence from a first species with a Needleman-Wunsch score greater than 800 when BLOSUM62 matrix, a gap opening penalty of 11, and a gap extension penalty of 1 are used; and
a second portion having an amino acid sequence that aligns with a functional segment of an ADAMTS12 CD domain amino acid sequence from a second species with a Needleman-Wunsch score greater than 1000 when BLOSUM62 matrix, a gap opening penalty of 11, and a gap extension penalty of 1 are used
wherein optionally the first species is rat, and the second species is human.

13. A recombinant nucleic acid for expression of an ADAMTS-12 polypeptide, wherein the recombinant nucleic acid encodes for a recombinant polypeptide that comprises
a first portion having an amino acid sequence that aligns with a functional segment of an ADAMTS-12 prodomain amino acid sequence from a first species with a Needleman-Wunsch score greater than 800 when BLOSUM62 matrix, a gap opening penalty of 11, and a gap extension penalty of 1 are used; and
a second portion having an amino acid sequence that aligns with a functional segment of an ADAMTS12 catalytic domain amino acid sequence from a second species with a Needleman-Wunsch score greater than 700 when BLOSUM62 matrix, a gap opening penalty of 11, and a gap extension penalty of 1 are used
wherein optionally the first species is rat, and the second species is human.

14. The recombinant nucleic acid of any one of claims 8 to 13, wherein the second portion comprises an E397Q mutation with respect to the amino acid sequence of SEQ ID NO: 15.

15. The recombinant nucleic acid of any of claims 1 to 14, wherein the encoded polypeptide or the second portion thereof is suited to cleave a peptide comprising standard residues 1-15 of the amino acid sequence of SEQ ID NO: 4, preferably with a kcat/KM of at least 20% of a corresponding kcat/KM of human ADAMTS-7 or human ADAMTS-12.

16. A recombinant polypeptide encoded by the recombinant nucleic acid according to any of claims 1 to 14, or a fragment of that recombinant polypeptide, wherein said recombinant polypeptide or fragment thereof is suited to cleave a peptide substrate comprising standard residues 1-15 of SEQ ID NO: 4.

17. A peptide substrate for ADAMTS-7 and/or ADAMTS-12, the peptide substrate comprising
a. residues 1-15 of the amino acid sequence of SEQ ID NO: 4, or
b. residues 1-15 of the amino acid sequence of SEQ ID NO: 5, or
c. residues 1-13 of the amino acid sequence of SEQ ID NO: 8, or
d. a fragment of any of the sequences according to a), b) or c), the fragment comprising the amino acid sequence EL.

18. The peptide substrate of claim 17, wherein the peptide substrate comprises a first moiety conjugated to a residue that is N-terminal to sequence fragment EL comprised within SEQ ID NO: 4,5, or 8 or the fragment thereof, and a second moiety conjugated to a residue that is C-terminal to said sequence fragment EL.

19. The peptide substrate of claim 18, wherein the first moiety comprises a fluorophore and the second moiety comprises a quencher, or wherein the first moiety comprises a quencher and the second moiety comprises a fluorophore.

20. A method for the identification or characterization of an ADAMTS-7 and/or ADAMTS-12 modulator comprising the steps of
a. contacting a recombinant polypeptide or a fragment thereof according to claim 12 with at least one test compound;
b. contacting said recombinant polypeptide or fragment thereof with a peptide substrate according to any one of claims 13 to 15, wherein the peptide substrate comprises a fluorophore and a quencher; and
c. detecting fluorescence as a measure for the activity of said recombinant polypeptide or a fragment thereof.

21. A method of producing a recombinant polypeptide according to claim 16, the method comprising
a. cultivating a recombinant host cell comprising a recombinant nucleic acid according to any of claims 1 to 11
b. recovering the recombinant polypeptide of a fragment thereof according to claim 12.

22. Kit of parts comprising at least one recombinant nucleic acid according to any of claims 1 to 15 or at least one polypeptide according to claim 16 and a peptide substrate according to any of claims 17 to 19.


SEQUENCE LISTING (48 sequences)

SEQ ID NO: 1 - protein, 562 aa, Artificial Sequence
  rPro-hCD (Rat 1-217/Human 237-537)-TEV-2Strep- 6His
  ADAMTS7 rat prodomain (wild-type) (1)..(217)
  ADAMTS7 human catalytic domain (218)..(518)
  TEV cleavage site, 2xStrep tag, and a His tag (519)..(562)
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 2 - protein, 562 aa, Artificial Sequence
  rPro-hCD-FM2 (Rat 1-217/Human 237-537 FM2 (Q216K))-TEV-2Strep-6His
  ADAMTS7 rat prodomain (furin mutant Q216K) (1)..(217)
  ADAMTS7 human catalytic domain (218)..(518)
  TEV cleavage site, 2xStrep tag, and a His tag (519)..(562)
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQKRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 3 - protein, 590 aa, Artificial Sequence
  rADAMTS7 (Rat 1-575)-Flag
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQRSISKEKWVETLVVADSKMVEYHGQPQVESYVLTIMNMVAGLYHDPSIGNPIHITVVRLIILEDEEKDLKITHHADDTLKNFCRWQKNVNMKGDDHPQHHDTAILLTRKDLCATMNHPCETLGLSHVAGLCHPQLSCSVSEDTGLPLAFTVAHELGHSFGIQHDGTGNDCESIGKRPFIMSPQLLYDRGIPLTWSRCSREYITRFLDRGWGLCLDDRPSKGVINFPSVLPGVLYDVNHQCRLQYGPSSAYCEDVDNVCYTLWCSVGTTCHSKMDAAVDGTSCGKNKWCLNGECVPEGFQPETVDGGWSGWSAWSVCSRSCGVGVRSSERQCTQPVPKNKGKYCVGERKRYRLCNLQACPENLYFQGDYKDDDDK

SEQ ID NO: 4 - protein, 16 aa, Artificial Sequence
  Test substrate for ADAMTS7/12 (HiLyteFluor- 488)-DELSSMVLELRGLRT-K(QXL520)-NH2
DELSSMVLELRGLRTK

SEQ ID NO: 5 - protein, 16 aa, Artificial Sequence
  Test Substrate for ADAMTS7/12 (HiLyteFluor-488)-SSMVLELRGLRTIVT-K(QXL520)-NH2
SSMVLELRGLRTIVTK

SEQ ID NO: 6 - protein, 16 aa, Artificial Sequence
  Test Substrate for ADAMTS7/12 (HiLyteFluor-488)-KVTEENKELANELRR-K(QXL520)-NH2
KVTEENKELANELRRK

SEQ ID NO: 7 - protein, 16 aa, Artificial Sequence
  Test Substrate for ADAMTS7/12 (HiLyteFluor-488)-EENKELANELRRPPL-K(QXL520)-NH2
EENKELANELRRPPLK

SEQ ID NO: 8 - protein, 13 aa, Artificial Sequence
  Test Substrate for ADAMTS7/12 (HiLyteFluor-488)-SSMVLELRGLRT-K(QXL520)-NH2
SSMVLELRGLRTK

SEQ ID NO: 9 - protein, 12 aa, Artificial Sequence
  Test Substrate for ADAMTS7/12 (Donor) GMQQSVRTGLPS (K-Quencher)
GMQQSVRTGLPS

SEQ ID NO: 10 - protein, 14 aa, Artificial Sequence
  Test Substrate for ADAMTS7/12 (Donor) SPGFRCEACPPGYS (K-Quencher)
SPGFRCEACPPGYS

SEQ ID NO: 11 - protein, 16 aa, Artificial Sequence
  Substrate for ADAMTS7 and for ADAMTS12 (HiLyteFluor-488)-DELSSMVLELRGLRT-K(QXL520)-E-NH2
DELSSMVLELRGLRTK

SEQ ID NO: 12 - protein, 16 aa, Artificial Sequence
  Test Substrate for ADAMTS7/12 (HiLyteFluor-488)-DELSSMVLELRGLRT-K(QXL520)-K-NH2
DELSSMVLELRGLRTK

SEQ ID NO: 13 - protein, 16 aa, Artificial Sequence
  Test Substrate for ADAMTS7/12 (HiLyteFluor-488)-DELSSMVLELRGLRT-K(QXL520)-OH
DELSSMVLELRGLRTK

SEQ ID NO: 14 - protein, 10 aa, Artificial Sequence
  Substrate for ADAMTS4 and for ADAMTS5 Dabcyl-EEVKAKVQPY-Glu(Edans)-NH2
EEVKAKVQPY

SEQ ID NO: 15 - protein, 573 aa, Artificial Sequence
  rPro-hCD for ADAMTS12
MPCAQGNWMAKLSMVAQLLNFGAFCHGRQAQPWPVRFPDPKQEHFIKSLPEYHIVSPVQVDASGHFLSYGLHHPVTGSRKKRAAGGSGDQVYYRISHEEKNLFFNLTVNWEFLSNGYVVERRYGNLSHVKMAASSGQPCHLRGTVLQQGPTIRMGTAALSACQGLTGFFHLPHGDFFIEPVKKHPLTEEGYQPHVIYRRQSYRVPETKEPTCGLKDSLDNSVKQELQREKWERKNWPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGGGSDYKDHDGDYKDHDIDYKDDDDK

SEQ ID NO: 16 - protein, 14 aa, Artificial Sequence
  ADAMTS Catalytic Motif Consensus Sequence
  misc_feature (3)..(4): Xaa can be any naturally occurring amino acid
  misc_feature (6)..(6): Xaa can be any naturally occurring amino acid
  misc_feature (12)..(12): Xaa can be any naturally occurring amino acid
HEXXHXBGNSBXHD

SEQ ID NO: 17 - protein, 7 aa, Artificial Sequence
  Substrate for MMP12 Mca-Pro-Leu-Gly-Leu-Glu- Glu-Ala-Dap(Dnp)-NH2
PLGLEEA

SEQ ID NO: 18 - protein, 8 aa, Artificial Sequence
  Substrate for MMP15 MCA-Lys-Pro-Leu-Gly-Leu- DPA-Ala-Arg-NH2
  X (6)..(6): DPA
KPLGLXAR

SEQ ID NO: 19 - protein, 7 aa, Artificial Sequence
  Substrate for MMP2 MCA-Pro-Leu-Gly-Leu-DPA- Ala-Arg-NH2
  X (5)..(5): DPA
PLGLXAR

SEQ ID NO: 20 - protein, 12 aa, Artificial Sequence
  Substrate for ADAM17 Mca-Pro-Leu-Ala-Gln-Ala-Val-Dap(Dnp)-Arg-Ser-Ser-Ser-Arg-NH2
  X (7)..(7): Dap(Dnp)
PLAQAVXRSSSR

SEQ ID NO: 21 - protein, 1686 aa, Artificial Sequence
  Human ADAMTS7 from Q9UKP4
MPGGPSPRSPAPLLRPLLLLLCALAPGAPGPAPGRATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSHPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELWPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNAGWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSGPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR

SEQ ID NO: 22 - protein, 1595 aa, Artificial Sequence
  Rat ADAMTS7 from Q1EHB3
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQRSISKEKWVETLVVADSKMVEYHGQPQVESYVLTIMNMVAGLYHDPSIGNPIHITVVRLIILEDEEKDLKITHHADDTLKNFCRWQKNVNMKGDDHPQHHDTAILLTRKDLCATMNHPCETLGLSHVAGLCHPQLSCSVSEDTGLPLAFTVAHELGHSFGIQHDGTGNDCESIGKRPFIMSPQLLYDRGIPLTWSRCSREYITRFLDRGWGLCLDDRPSKGVINFPSVLPGVLYDVNHQCRLQYGPSSAYCEDVDNVCYTLWCSVGTTCHSKMDAAVDGTSCGKNKWCLNGECVPEGFQPETVDGGWSGWSAWSVCSRSCGVGVRSSERQCTQPVPKNKGKYCVGERKRYRLCNLQACPPDRPSFRHTQCSQFDSMLYKGKLHKWVPVLNDENPCELHCRPFNYSNREKLRDAVMDGTPCYQGRISRDICIDGICKKVGCDFELDSGAEEDRCGVCRGDGSTCHTVSRTFKEAEGMGYVDVGLIPAGAREILIEEVAEAANFLALRSEDPDKYFLNGGWTIQWNGDYQVAGTTFTYTRKGNWETLTSPGPTTEPVWIQLLFQERNPGVHYKYTIQRASHSEAQPPEFSWHYGPWSKCPVTCGTGVQRQSLYCMEKQAGIVDEGHCDHLSRPRDRKRKCNEEPCPARWWVGDWQPCSRSCGPGGFFRRAVFCTRSVGLDEQRALEPSACGHLPRPLAEIPCYHYVACPSSWGVGNWSQCSVTCGAGIRQRSVLCINNTGVPCDGAERPITETFCFLQPCQYSTYIVDTGASGSGSSSPELFNEVDFDPHQPVPRPSPASSPKPVSISNAIDEEDPELDPPGPVFVDDFYYDYNFINFHEDLSYGSFEESHSDLVDIGGQTVPPHIRPTEPPSDSPVPTAGAPGAEEEGIQGSWSPSPLLSEASHSPPVLLENTPVNPLANFLTEEESPIGAPELGLPSVSWPPASVDGMVTSVAPGNPDELLVREDTQSQPSTPWSDRNKLSKDGNPLGPTSPALPKSPFPTQPSSPSNSTTQASLSPDAVEVSTGWNVALDPVLEADLKPVHAPTDPGLLDQIQTPHTEGTQSPGLLPRPAQETQTNSSKDPAVQPLQPSLVEDGAPTDLLPAKNASWQVGNWSQCSTTCGLGAIWRLVRCSSGNDEDCTLSSRPQPARHCHLRPCAAWRAGNWSKCSRNCGGGSATRDVQCVDTRDLRPLRPFHCQPGPTKPPTRQLCGTQPCLPWYTSSWRECSEACGGGEQQRLVTCPEPGLCEESLRPNNTRPCNTHPCTQWVVGPWGQCSAPCGGGVQRRLVKCVNTQTGLAEEDSDLCSHEAWPESSRPCATEDCELVEPSRCERDRLPFNFCETLRLLGRCQLPTIRAQCCRSCPPLSRGVPSRGHQRVARR

SEQ ID NO: 23 - protein, 1657 aa, Artificial Sequence
  Mouse ADAMTS7 from Q68SA9
MHRGPSLLLILCALASRVLGPASGLVTEGRAGLDIVHPVRVDAGGSFLSYELWPRVLRKRDVSTTQASSAFYQLQYQGRELLFNLTTNPYLMAPGFVSEIRRHSTLGHAHIQTSVPTCHLLGDVQDPELEGGFAAISACDGLRGVFQLSNEDYFIEPLDGVSAQPGHAQPHVVYKHQGSRKQAQQGDSRPSGTCGMQVPPDLEQQREHWEQQQQKRRQQRSVSKEKWVETLVVADSKMVEYHGQPQVESYVLTIMNMVAGLFHDPSIGNPIHISIVRLIILEDEEKDLKITHHAEETLKNFCRWQKNINIKGDDHPQHHDTAILLTRKDLCASMNQPCETLGLSHVSGLCHPQLSCSVSEDTGMPLAFTVAHELGHSFGIQHDGTGNDCESIGKRPFIMSPQLLYDRGIPLTWSRCSREYITRFLDRGWGLCLDDRPSKDVIALPSVLPGVLYDVNHQCRLQYGSHSAYCEDMDDVCHTLWCSVGTTCHSKLDAAVDGTSCGKNKWCLKGECVPEGFQPEAVDGGWSGWSAWSDCSRSCGVGVRSSERQCTQPVPKNRGKYCVGERKRSQLCNLPACPPDRPSFRHTQCSQFDGMLYKGKLHKWVPVPNDDNPCELHCRPSNSSNTEKLRDAVVDGTPCYQSRISRDICLNGICKNVGCDFVIDSGAEEDRCGVCRGDGSTCQTVSRTFKETEGQGYVDIGLIPAGAREILIEEVAEAANFLALRSEDPDKYFLNGGWTIQWNGDYRVAGTTFTYARKGNWENLTSPGPTSEPVWIQLLFQEKNPGVHYQYTIQRDSHDQVRPPEFSWHYGPWSKCTVTCGTGVQRQSLYCMERQAGVVAEEYCNTLNRPDERQRKCSEEPCPPRWWAGEWQPCSRSCGPEGLSRRAVFCIRSMGLDEQRALELSACEHLPRPLAETPCNRHVICPSTWGVGNWSQCSVTCGAGIRQRSVLCINNTDVPCDEAERPITETFCFLQPCQYPMYIVDTGASGSGSSSPELFNEVDFIPNQLAPRPSPASSPKPVSISNAIDEEELDPPGPVFVDDFYYDYNFINFHEDLSYGSFEEPHPDLVDNGGWTAPPHIRPTESPSDTPVPTAGALGAEAEDIQGSWSPSPLLSEASYSPPGLEQTSINPLANFLTEEDTPMGAPELGFPSLPWPPASVDDMMTPVGPGNPDELLVKEDEQSPPSTPWSDRNKLSTDGNPLGHTSPALPQSPIPTQPSPPSISPTQASPSPDVVEVSTGWNAAWDPVLEADLKPGHGELPSTVEVASPPLLPMATVPGIWGRDSPLEPGTPTFSSPELSSQHLKTLTMPGTLLLTVPTDLRSPGPSGQPQTPNLEGTQSPGLLPTPARETQTNSSKDPEVQPLQPSLEEDGDPADPLPARNASWQVGNWSQCSTTCGLGAIWRLVSCSSGNDEDCTLASRPQPARHCHLRPCAAWRTGNWSKCSRNCGGGSSTRDVQCVDTRDLRPLRPFHCQPGPTKPPNRQLCGTQPCLPWYTSSWRECSEACGGGEQQRLVTCPEPGLCEESLRPNNSRPCNTHPCTQWVVGPWGQCSAPCGGGVQRRLVRCVNTQTGLAEEDSDLCSHEAWPESSRPCATEDCELVEPPRCERDRLSFNFCETLRLLGRCQLPTIRAQCCRSCPPLSRGVPSRGHQRVARR

SEQ ID NO: 24 - protein, 1594 aa, Artificial Sequence
  Human ADAMTS12 from P58397
MPCAQRSWLANLSVVAQLLNFGALCYGRQPQPGPVRFPDRRQEHFIKGLPEYHVVGPVRVDASGHFLSYGLHYPITSSRRKRDLDGSEDWVYYRISHEEKDLFFNLTVNQGFLSNSYIMEKRYGNLSHVKMMASSAPLCHLSGTVLQQGTRVGTAALSACHGLTGFFQLPHGDFFIEPVKKHPLVEGGYHPHIVYRRQKVPETKEPTCGLKDSVNISQKQELWREKWERHNLPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGWGRWSPWSHCSRTCGAGVQSAERLCNNPEPKFGGKYCTGERKRYRLCNVHPCRSEAPTFRQMQCSEFDTVPYKNELYHWFPIFNPAHPCELYCRPIDGQFSEKMLDAVIDGTPCFEGGNSRNVCINGICKMVGCDYEIDSNATEDRCGVCLGDGSSCQTVRKMFKQKEGSGYVDIGLIPKGARDIRVMEIEGAGNFLAIRSEDPEKYYLNGGFIIQWNGNYKLAGTVFQYDRKGDLEKLMATGPTNESVWIQLLFQVTNPGIKYEYTIQKDGLDNDVEQQMYFWQYGHWTECSVTCGTGIRRQTAHCIKKGRGMVKATFCDPETQPNGRQKKCHEKACPPRWWAGEWEACSATCGPHGEKKRTVLCIQTMVSDEQALPPTDCQHLLKPKTLLSCNRDILCPSDWTVGNWSECSVSCGGGVRIRSVTCAKNHDEPCDVTRKPNSRALCGLQQCPSSRRVLKPNKGTISNGKNPPTLKPVPPPTSRPRMLTTPTGPESMSTSTPAISSPSPTTASKEGDLGGKQWQDSSTQPELSSRYLISTGSTSQPILTSQSLSIQPSEENVSSSDTGPTSEGGLVATTTSGSGLSSSRNPITWPVTPFYNTLTKGPEMEIHSGSGEEREQPEDKDESNPVIWTKIRVPGNDAPVESTEMPLAPPLTPDLSRESWWPPFSTVMEGLLPSQRPTTSETGTPRVEGMVTEKPANTLLPLGGDHQPEPSGKTANRNHLKLPNNMNQTKSSEPVLTEEDATSLITEGFLLNASNYKQLTNGHGSAHWIVGNWSECSTTCGLGAYWRRVECSTQMDSDCAAIQRPDPAKRCHLRPCAGWKVGNWSKCSRNCSGGFKIREIQCVDSRDHRNLRPFHCQFLAGIPPPLSMSCNPEPCEAWQVEPWSQCSRSCGGGVQERGVFCPGGLCDWTKRPTSTMSCNEHLCCHWATGNWDLCSTSCGGGFQKRTVQCVPSEGNKTEDQDQCLCDHKPRPPEFKKCNQQACKKSADLLCTKDKLSASFCQTLKAMKKCSVPTVRAECCFSCPQTHITHTQRQRRQRLLQKSKEL

SEQ ID NO: 25 - protein, 1599 aa, Artificial Sequence
  Rat ADAMTS12 from D3ZTJ3
MPCAQGNWMAKLSMVAQLLNFGAFCHGRQAQPWPVRFPDPKQEHFIKSLPEYHIVSPVQVDASGHFLSYGLHHPVTGSRKKRAAGGSGDQVYYRISHEEKNLFFNLTVNWEFLSNGYVVERRYGNLSHVKMAASSGQPCHLRGTVLQQGPTIRMGTAALSACQGLTGFFHLPHGDFFIEPVKKHPLTEEGYQPHVIYRRQSYRVPETKEPTCGLKDSLDNSVKQELQREKWERKNWPSRSLSRRSISKERWVETLVVADTKMVEYHGSENVESYILTIMNMVTGLFHNPSIGNAVHIVVVRLILLEEEEQGLKIVHHAEKTLSSFCKWQKSINPKSDLNPVHHDVAVLITRKDICAGVNRPCETLGLSQLSGMCQPHRSCNINEDSGLPLAFTIAHELGHSFGIQHDGKENDCEPVGRHPYIMSQQIQYDPTPLTWSKCSKEYITRFLDRGRGFCLDDVPRKKGLKSNVIAPGVIYDVHHQCQLQYGPNATFCQEVENVCQTLWCSVKGFCRSKLDAAADGTRCGEKKWCMAGKCITVGKKPESIPGGWGRWSPWSHCSRTCGAGAQSAERLCNNPEPKFGGKYCTGERKRYRLCNVHPCRSDTPTFRQMQCSEFDTVPYKNQFYRWFPVFNPAHPCELYCRPIDEQFSERMLEAVIDGTPCFEGGNSRNVCINGICKRVGCDYEIDSNATEDRCGVCLGDGSACQTVKKVFRQKEGSGYIDIGLIPKGARDIRVMEIKAAGNFLAIRSEDPEKYYLNGGFIIQWNGNYKLAGTVFQYDRKGDLERLMAPGPTNESVWLQLLFQVTNPGIKYEYTVRKDGLDNDVEKLLYFWQFGRWTECSVTCGTGIRRQTAHCVKKGHGIVKTTFCNPETQPSVRQKKCYEKDCPPRWWAGEWEACSMTCGPYGEKKRTVLCIQTMGSDEQALPATDCQHLLKPKTLVSCNRDILCPSDWTVGNWSECSVSCGGGVRIRSVTCAKNLNEPCDKTRKPNSRALCGLQQCPFSRRVLKPNKDTVPSGKNPTTSEHDHFKPIPASTSRPTPLSTPTVPESVSTSTPTINSLGSTITSQEEPDGIGWQNNSTQAEEDSHIPTSVGSTSQTPLTSWSWSMQPDDENVSSSAIGPTSESDFWATTSDSGLSSSNAMTWQVTPFYSTATTEPEVEIHSGSGEDSDQPLNKEENNSVLWNKIRVPERDAPMEMDAEIPLGPPPTSYVTEESSWPPFSTMMKSSLPAWSFKNETPRDEGMITEKSGNIPLPLGGDHQTTSPEKLGNNDQLASANSTNPTQGSGPVLTEEDASTLIEEGFLLNASNYKHLMKDHSPAHWIVGNWSKCSTTCGLGAYWRSVECSTGMNADCAAIQRPDPAKKCHLRPCAGWRVGNWSKCSRNCSGGFKIREVQCMDGVDHHRSLRPFHCQFLAGVPPPLSMSCNLEPCEEWKVEPWSQCSRSCGGGVQERGVFCPGGLCDWTKRPASTVPCNRHLCCHWATGNWELCTTSCGGGSQKRTVHCIPSENSTTEDQDQCFCDHQARPPEFQNCNQQACRKSADLTCTKDRLSTSFCQTLKSMKKCSVPSVRVQCCLSCPQTQSIHTQRQRKQQMLQNHDTL

SEQ ID NO: 26 - protein, 1600 aa, Artificial Sequence
  Mouse ADAMTS12 from Q811B3
MPCARGSWLAKLSIVAQLINFGAFCHGRQTQPWPVRFPDPRQEHFIKSLPEYHIVSPVQVDAGGHVLSYGLHHPVTSSRKKRAAGGSGDQLYYRISHEEKDLFFNLTVNWEFLSNGYVVEKRYGNLSHVKMVASSGQPCHLRGTVLQQGTTVGIGTAALSACQGLTGFFHLPHGDFFIEPVKKHPLTEEGSYPHVVYRRQSIRAPETKEPICGLKDSLDNSVKQELQREKWERKTLRSRSLSRRSISKERWVETLVVADTKTVEYHGSENVESYILTIMNMVTGLFHSPSIGNLVHIVVVRLILLEEEEQGLKIVHHAEKTLSSFCKWQKSINPKSDLNPVHHDVAVLITRKDICAGVNRPCETLGLSQLSGMCQPHRSCNINEDSGLPLAFTIAHELGHSFGIQHDGKENDCEPVGRHPYIMSQQIQYDPTPLTWSKCSKEYITRFLDRGRGFCLDDIPSKKGLKSNVIAPGVIYDVHHQCQLQYGPNATFCQEVENVCQTLWCSVKGFCRSKLDAAADGTRCGEKKWCMAGKCITVGKKPESIPGGWGRWSPWSHCSRTCGAGAQSAERLCNNPEPKFGGKYCTGERKRYRLCNVHPCRSDTPTFRQMQCSEFDTVPYKNQFYRWFPVFNAAHPCELYCRPIDEQFSERMLEAVIDGTPCFEGGNSRNVCINGICKRVGCDYEIDSNATEDRCGVCLGDGSACQTVKKLFRQKEGSGYVDIGLIPKGARDIRVMEIKAAGNFLAIRSEDPEKYYLNGGFIIQWNGNYKLAGTVFQYDRKGDLEKLIAPGPTNESVWLQLLFQVTNPGIKYEYTVRKDGLDNDVEKLLYFWQFGRWTECSVTCGTGIRRQAAHCVKKGHGIVKTTFCNPETQPSVRQKKCHEKDCPPRWWAGEWEACSTTCGPYGEKKRTVLCIQTMGSDEQALPATDCQHLLKPKALVSCNRDILCPSDWTVGNWSECSVSCGGGVRIRSVTCAKNLNEPCDKTRKPNSRALCGLQQCPFSRRVLKPNKDIAPSGKNQSTAEHDPFKPIPAPTSRPTPLSTPTVPESMSTSTPTINSLGSTIASQEDANGMGWQNNSTQAEEGSHFPTSSGSTSQVPVTSWSLSIQPDDENVSSSAIGPTSEGDFWATTTSDSGLSSSDAMTWQVTPFYSTMTTDPEVEIHSGSGEDSDQPLNKDKSNSVIWNKIGVPEHDAPMETDAELPLGPPPTSYMGEEPSWPPFSTKMEGSLPAWSFKNETPRDDGMIAEKSRKIPLPLAGDHHPATSEKLENHDKLALPNTTNPTQGFGPVLTEEDASNLIAEGFLLNASDYKHLMKDHSPAYWIVGNWSKCSTTCGLGAYWRSVECSSGVDADCTTIQRPDPAKKCHLRPCAGWRVGNWSKCSRNCSGGFKIREVQCMDSLDHHRSLRPFHCQFLAGAPPPLSMSCNLEPCGEWQVEPWSQCSRSCGGGVQERGVSCPGGLCDWTKRPATTVPCNRHLCCHWATGNWELCNTSCGGGSQKRTIHCIPSENSTTEDQDQCLCDHQVKPPEFQTCNQQACRKSADLTCLKDRLSISFCQTLKSMRKCSVPSVRAQCCLSCPQAPSIHTQRQRKQQLLQNHDML

SEQ ID NO: 27 - protein, 202 aa, Artificial Sequence
  Human ADAMTS7 Pro Domain (hADAMTS7_Pro)
RATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQR

SEQ ID NO: 28 - protein, 193 aa, Artificial Sequence
  Rat ADAMTS7 Pro Domain (rADAMTS7_Pro)
LPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQR

SEQ ID NO: 29 - protein, 301 aa, Artificial Sequence
  Human ADAMTS7 CD domain (hADAMTS7_CD)
SVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAV

SEQ ID NO: 30 - protein, 302 aa, Artificial Sequence
  Rat ADAMTS7 CD domain (rADAMTS7_CD)
SISKEKWVETLVVADSKMVEYHGQPQVESYVLTIMNMVAGLYHDPSIGNPIHITVVRLIILEDEEKDLKITHHADDTLKNFCRWQKNVNMKGDDHPQHHDTAILLTRKDLCATMNHPCETLGLSHVAGLCHPQLSCSVSEDTGLPLAFTVAHELGHSFGIQHDGTGNDCESIGKRPFIMSPQLLYDRGIPLTWSRCSREYITRFLDRGWGLCLDDRPSKGVINFPSVLPGVLYDVNHQCRLQYGPSSAYCEDVDNVCYTLWCSVGTTCHSKMDAAVDGTSCGKNKWCLNGECVPEGFQPETV

SEQ ID NO: 31 - protein, 562 aa, Artificial Sequence
  rPro-hCD (Rat 1-217/Human 237-537 (R58A/R60A/R217A))-TEV-2Strep-6His
MHRGLNLLLILCALAPHVLGPASGLPTEGRAGLDIVHPVRVDAGGSFLSYELWPRVLAKADVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQASVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 32 - protein, 196 aa, Artificial Sequence
  Mouse ADAMTS-7 prodomain (Mouse_Pro)
LVTEGRAGLDIVHPVRVDAGGSFLSYELWPRVLRKRDVSTTQASSAFYQLQYQGRELLFNLTTNPYLMAPGFVSEIRRHSTLGHAHIQTSVPTCHLLGDVQDPELEGGFAAISACDGLRGVFQLSNEDYFIEPLDGVSAQPGHAQPHVVYKHQGSRKQAQQGDSRPSGTCGMQVPPDLEQQREHWEQQQQKRRQQR

SEQ ID NO: 33 - protein, 45 aa, Artificial Sequence
  TSP-1 candidate region
CDELSSMVLELRGLRTIVTTLQDSIRKVTEENKELANELRRPPLC

SEQ ID NO: 34 - DNA, 20 bp, Artificial Sequence
  3′loxP F for 3 primer PCR assay
tagaatagcgggctctcgtg

SEQ ID NO: 35 - DNA, 20 bp, Artificial Sequence
  LacZ F for 3 primer PCR assay
tttccatatggggattggtg

SEQ ID NO: 36 - DNA, 20 bp, Artificial Sequence
  3′loxP R for 3 primer PCR assay
ctcgggaatggaatcttgac

SEQ ID NO: 37 - DNA, 20 bp, Artificial Sequence
  Guide Sequence
ttacctgtgtcctagctcgt

SEQ ID NO: 38 - DNA, 132 bp, Artificial Sequence
  PAM Site
tgggcctgtctcatgtgtctggcttgtgccaccctcagctcagctgtagcgtcagtgaggatactggcatgccactggccttcactgtggctcaccagctaggacacaggtaactcatcttccctcacctct

SEQ ID NO: 39 - DNA, 20 bp, Artificial Sequence
  Adamts7 in06F
ccttctgaggtcccagtgag

SEQ ID NO: 40 - DNA, 50 bp, Artificial Sequence
  Adamts7 ex07Rp
ctgcaggaattcgatatcaagcttatcgatacctgtgtcctagctcgtgg

SEQ ID NO: 41 - DNA, 19 bp, Artificial Sequence
  Mut Adamts7 E373Q R
cctgtgtcctagctggtga

SEQ ID NO: 42 - DNA, 20 bp, Artificial Sequence
  Noc41 region
caccctgtgtcctagctcct

SEQ ID NO: 43 - DNA, 20 bp, Artificial Sequence
  Zfp446 region
gaacctgtgtcccagctctt

SEQ ID NO: 44 - DNA, 20 bp, Artificial Sequence
  Ncoa2 region
tcacctgggtccgagctggt

SEQ ID NO: 45 - DNA, 20 bp, Artificial Sequence
  Drosha region
ctacctgtttcctagcttgg

SEQ ID NO: 46 - DNA, 22 bp, Artificial Sequence
  Adamts7 ex05 F
cgatcatgaacatggtggctgg

SEQ ID NO: 47 - DNA, 22 bp, Artificial Sequence
  Adamts7 ex08 R
tggacggtcatctaagcacagg

SEQ ID NO: 48 - DNA, 20 bp, Artificial Sequence
  Adamts7 in07 R
actccctcaggtttcgtgac